(19) United States
(12) Patent Application Publication
(10) Pub. No.: US 2014/0200678 A1
(43) Pub. Date: Jul. 17, 2014

(54) BIOMATERIALS WITH MICROSPHERE GRADIENTS AND CORE AND SHELL MICROSPHERES

(71) Applicants: Michael Detamore, Lawrence, KS (US); Vineet Gupta, Lawrence, KS (US); Neethu Mohan, Lawrence, KS (US); Cory Berkland, Lawrence, KS (US)

(72) Inventors: Michael Detamore, Lawrence, KS (US); Vineet Gupta, Lawrence, KS (US); Neethu Mohan, Lawrence, KS (US); Cory Berkland, Lawrence, KS (US)

(73) Assignee: THE UNIVERSITY OF KANSAS, Lawrence, KS (US)

(21) Appl. No.: 14/202,379

(22) Filed: Mar. 10, 2014

Related U.S. Application Data
(63) Continuation-in-part of application No. 13/591,087, filed on Aug. 21, 2012, now Pat. No. 8,669,107.
Application No. 13/591,087 is a division of application No. 12/248,530, filed on Oct. 9, 2008, now Pat. No. 8,277,832.
Application No. 12/248,530 is a continuation-in-part of application No. PCT/US2013/024457, filed on Feb. 3, 2013.
(60) Provisional application No. 61/594,568, filed on Feb. 3, 2012.

(51) Int. Cl. A61L 27/56; A61L 27/38

(52) U.S. Cl. CPC A61L 27/56; A61L 27/38
USPC 623/23.73

(57) ABSTRACT

Methods can prepare tissue engineering scaffolds that include a plurality of biocompatible core/shell microspheres linked together to form a three-dimensional matrix. The matrix can include a plurality of pores for growing cells. The biocompatible microspheres can include first and second sets of microspheres. The first set of microspheres can have a first characteristic, and a first predetermined spatial distribution with respect to the three-dimensional matrix. The second set of microspheres can have a second characteristic that is different from the first characteristic, and a second predetermined spatial distribution that is different from the first predetermined spatial distribution with respect to the three-dimensional matrix. The first and second characteristics can selected a composition, polymer, particle size, particle size distribution, type of bioactive agent, type of bioactive agent combination, bioactive agent concentration, amount of bioactive agent, rate of bioactive agent release, mechanical strength, flexibility, rigidity, color, radiotranslucency, radiopaqueness, or the like.

### CROSS-REFERENCE

This patent application is a continuation-in-part of U.S. patent application Ser. No. 13/591,087 filed Aug. 21, 2013 now U.S. Pat. No. 8,669,107, which is a divisional application of U.S. patent application Ser. No. 12/248,530 filed Oct. 9, 2008 now U.S. Pat. No. 8,277,832, and this patent application is a continuation-in-part of PCT Patent Application Serial No. PCT/US13/24457 filed Feb. 1, 2013, which claims the benefit of U.S. Provisional Patent Application No. 61/594,568 filed Feb. 3, 2012. These patent applications and patent are incorporated herein by specific reference in their entirety.

### GOVERNMENT RIGHTS

This invention was made with government support under R01 AR056347 awarded by the National Institutes of Health. The government has certain rights in the invention.

### BACKGROUND

Spatial patterning of biological cues is of special interest to investigators in areas such as nerve tissue engineering, study of chemotaxis, etc., where gradients of surface-immobilized or soluble signaling molecules have been employed. To modulate the immune and/or inflammatory systems, controlled spatial and temporal release of anti-inflammatories or chemokines are desired. In addition, interfacial tissue engineering is another area that may benefit from scaffolds with biphasic and gradient distributions of another area that may benefit from scaffolds with biphasic and gradient distributions of bioactive signals. Previous diffusion- and convection-driven approaches for the generation of linear or non-linear signal gradients are simple and inexpensive, however restricted to the generation of limited set of profiles. Use of photolithographic and soft lithographic techniques (e.g., microcontact printing and microfluidics) can provide micron-level positional accuracy; however, such techniques are expensive and largely limited to two-dimensional constructs. Commercially available gravity- and motor-driven gradient makers (Gradient maker, CBS Scientific, CA; Gradient former, Jule, Inc., CT), designed for applications in electrophoresis, have been utilized in some tissue engineering studies (Shoichet group, West group), but can only create certain gradient profiles, and have only been used to fabricate gel-based scaffolds. In addition, although some of the previous techniques have been used to create three-dimensional scaffolds with spatial gradients of bioactive agents (e.g., soluble growth factor), little attention has been paid towards the controlled temporal release aspect. Therefore it would be advantageous to have a three-dimensional scaffold for use as an endoprosthesis that can provide controlled spatial and temporal release of a suitable bioactive agent.

Engineered particles, such as nanospheres and microspheres, continue to gain importance for their use in a wide variety of industries. Continued improvement and modifications to engineered particles is sought to enhance current applications and for adaptation to new end uses. The engineered particles can be configured for broad application to a wide variety of technologies or to be tailored for a specific end use. Biotechnology applications of engineered particles, such as for implantation of microsphere tissue scaffolds, can benefit from having a range of different types of properties. For example, tissue scaffolds can be engineered for implantation into hard or soft tissue areas or for replacement of hard or soft tissues as well as for the interfaces therebetween. Thus, there is still a need to develop improved engineered particles with different characteristics for improved use in different applications.

### SUMMARY

The present invention includes three-dimensional tissue engineering scaffolds that can be used as endoprostheses. More particularly, the present invention relates to three-dimensional tissue engineering scaffolds that are prepared from microspheres having different characteristics.

In one embodiment, a tissue engineering scaffold for growing cells can include a plurality of biocompatible microspheres linked together so as to form a three-dimensional matrix. The matrix can include a plurality of pores defined by and disposed between the microspheres. Also, the microspheres can have a surface area sufficient for growing cells within the plurality of pores. The biocompatible microspheres can include first and second sets of microspheres. The first set of microspheres can have a first characteristic, and can have a first predetermined spatial distribution with respect to the three-dimensional matrix. The second set of microspheres can have a second characteristic that is different from the first characteristic, and can have a second predetermined spatial distribution that is different from the first predetermined spatial distribution with respect to the three-dimensional matrix. For example, the first and second characteristics can be independently selected from the group consisting of the following: composition; polymer; particle size; particle size distribution; type of bioactive agent; type of bioactive agent combination; bioactive agent concentration; amount of bioactive agent; rate of bioactive agent release; mechanical strength; flexibility; rigidity; color; radiotranslucency; radiopaqueness; or the like.

In one embodiment, the first predetermined spatial distribution of the microspheres can be distinct from and adjacent to the second predetermined spatial distribution of the microspheres. Also, the first predetermined spatial distribution of microspheres can form a first concentration gradient of the first set of microspheres, and the second predetermined spatial distribution of the microspheres can form a second concentration gradient of the second set of microspheres. Additionally, the first predetermined spatial distribution gradient and second predetermined spatial distribution gradient can blend into each other.

In one embodiment, the three dimensional matrix can include a first portion and a second portion. The first portion can have a majority of microspheres of the first set. The second portion can have a majority of microspheres of the second set. Optionally, a third portion of the three dimensional matrix can be disposed between the first portion and the second portion. The first predetermined spatial distribution in the third portion forms a first concentration gradient of the first set of microspheres and the second predetermined spatial distribution in the third portion forms a second concentration gradient of the second set of microspheres. Optionally, the first or second concentration gradient can be linear or nonlinear.

In one embodiment, the scaffold can include a first bioactive agent contained in or disposed on the first set of microspheres. The scaffold can be configured to release the first bioactive agent so as to create a first desired spatial and temporal concentration gradient of the first bioactive agent. Optionally, the second set of microspheres can be substantially devoid of the first bioactive agent, or can include a second bioactive agent. When the second bioactive agent is contained in or disposed on the second set of microspheres, the scaffold can be configured to release the second bioactive agent so as to create a second desired spatial and/or temporal concentration gradient of the second bioactive agent that is different from the first desired spatial and/or temporal concentration gradient of the first bioactive agent.

In one embodiment, the microspheres can be melded together by a portion of each microsphere merging with a portion of at least one adjacent microsphere. Methods of melding microspheres together are described herein. For example, a solvent, such as ethanol, can be used for melding.

In one embodiment, the bioactive agent contained in a microsphere can be a growth factor for growing the cells. However, the microspheres can include any type of bioactive agent. Accordingly, the first characteristic can be a first bioactive agent contained in or disposed on the microspheres, and the second characteristic can be a second bioactive agent contained in or disposed on the microspheres. For example, the first bioactive agent can be an osteogenic factor and the second bioactive agent can be a chondrogenic factor.

In one embodiment, at least one of the first set or second set of microspheres can include a biodegradable polymer. For example, the microspheres can include a poly-lactide-co-glycolide or poly(lactic-co-glycolic acid).

In one embodiment, the scaffold can include a medium sufficient for growing cells disposed in the pores. The medium can be a cell culture media. Additionally, the medium can be a body fluid or tissue.

In one embodiment, the scaffold can include a plurality of cells attached to the plurality of microspheres and growing within the pores. Such cells can include a first cell type associated with the first set of microspheres, and a second cell type associated with the second set of microspheres.

In one embodiment, the scaffold can include a third set of microspheres having a third characteristic that is the same or different from the first or second characteristics. The third set of microspheres can have a third predetermined spatial location that is different from the first or second predetermined spatial locations with respect to the three-dimensional matrix.

In one embodiment, the scaffold can include a first end and an opposite second end. Accordingly, the first set of microspheres can have a first bioactive agent, and the first end can have a majority of microspheres of the first set. Correspondingly, the second set of microspheres can have a second bioactive agent that is different from the first bioactive agent, and the second end having a majority of microspheres of the second set.

In one embodiment, the present invention can include a method of preparing tissue engineering scaffold for growing cells. Such a method can include the following: providing a first set of microspheres having a first characteristic; providing a second set of microspheres having a second characteristic that is different from the first characteristic; and linking the microspheres of the first set and second set together so as to form a three-dimensional matrix having a plurality of pores defined by and disposed between the microspheres. The plurality of microspheres can have a surface area sufficient for growing cells within the plurality of pores. The three-dimensional matrix can include a first set of microspheres having a first predetermined spatial distribution with respect to the three-dimensional matrix, and a second set of microspheres having a second predetermined spatial distribution that is different from the first predetermined spatial distribution with respect to the three-dimensional matrix.

In one embodiment, the process of linking the microspheres together can include melding the microspheres such that the microspheres are melded together by a portion of each microsphere merging with a portion of at least one adjacent microsphere.

In one embodiment, the method of preparing a microsphere-based scaffold can include any one of the following: preparing a first liquid suspension of the first set of microspheres; preparing a second liquid suspension of the second set of microspheres; introducing the first liquid suspension into a mold; introducing the second liquid suspension into the mold before, during, and/or after introducing the first liquid suspension into the mold; introducing a solvent into the mold such that the solvent melds the microspheres of the first set and second set together into the three-dimensional matrix; and/or removing the solvent from the melded microspheres.

In one embodiment, the present invention can include a method of generating or regenerating tissue in an animal, such as a human. The method can include providing an endoprosthesis for growing cells. The endoprosthesis can have a plurality of biocompatible microspheres linked together so as to form a three-dimensional matrix having a plurality of pores defined by and disposed between the microspheres. Accordingly, the endoprosthesis can include a microsphere-based scaffold. The plurality of microspheres can have a surface area sufficient for growing cells within the plurality of pores. The biocompatible microspheres can be characterized as described herein. Additionally, the method of generating or regenerating tissue can include implanting the endoprosthesis in the animal such that cells grow on the microspheres and within the pores. This process can be used to grow specific types of cells for growth of tissue, bone, cartilage, or the like.

In one embodiment, the method of generating or regenerating tissue can include any one of the following: introducing a cell culture media into the pores; introducing cells into the pores; and/or culturing the cells such that the cells attach to the microspheres and grow within the pores.

The microspheres can be core/shell microspheres, such as those with the hard core and polymeric shell.

These and other embodiments and features of the present invention will become more fully apparent from the following description and appended claims, or may be learned by the practice of the invention as set forth hereinafter.

### FIGURES

To further clarify the above and other advantages and features of the present invention, a more particular description of the invention will be rendered by reference to specific embodiments thereof which are illustrated in the appended drawings. It is appreciated that these drawings depict only illustrated embodiments of the invention and are therefore not to be considered limiting of its scope. The invention will be described and explained with additional specificity and detail through the use of the accompanying drawings in which:

FIG. 1 illustrates a schematic representation of an embodiment of a system for preparing microsphere-based scaffolds.

FIGS. 2A-2D are schematic representations of flow profiles of microspheres in forming a microsphere-based scaffold and can represent a microsphere gradient within a microsphere-based scaffold.

FIG. 3A is a graphical representation of the size of microspheres used in preparing a microsphere-based scaffold.

FIG. 3B is a scanning electron micrograph of external and internal structures of microspheres used in preparing a microsphere-based scaffold.

FIG. 4A is a photograph of a microsphere-based scaffold.

FIGS. 4B-4D are scanning electron micrographs of the microspheres and melding of microspheres of a microsphere-based scaffold.

FIGS. 5A-5C are pictures of cells within a microsphere-based scaffold, where FIG. 5A shows live cells, FIG. 5B shows dead cells, and FIG. 5C is a combination of FIGS. 5A-5B.

FIGS. 6A-6D are pictures of microsphere-based scaffolds having microsphere distribution properties with more than one type of microsphere.

FIGS. 6E-6H are the relative intensities associated with the microspheres of FIGS. 6A-6D.

FIG. 7A is a schematic representation of a microsphere-based scaffold having TGF-beta and BMP-2 so as to be 100% chondrogenic at one end and 100% osteogenic at the other end.

FIGS. 7B and 7C are graphical representations of possible percent chondrogenic and osteogenic gradients with respect to a microsphere-based scaffold.

FIG. 8 is a graph illustrating the elastic modulus with respect to microsphere size.

FIGS. 9A-9C are histological photographs of hUCMSCs cells from microsphere-based scaffolds as follows: FIG. 9A) collagen I, FIG. 9B) collagen II, and FIG. 9C) is a negative control.

FIG. 10 is a graph of a substance release profile from a microsphere-based scaffold.

FIGS. 11A-11C are pictures of the microsphere distribution and structure within a microsphere-based scaffold obtained by microcomputed tomography (μCT) imaging.

FIG. 12 is a micrograph of a microsphere having nanophase CaCO3 encapsulated (right) and a microsphere with minimal nanophase CaCO3 encapsulation (left).

FIG. 13 is a graph illustrating the modulus of elasticity of nanophase CaCO3 microspheres and nanophase TiO2 microspheres at increasing concentrations.

FIG. 14 is a graph illustrating the modulus of elasticity of a PLGA microsphere-based scaffold with 10% nanophase CaCO3 compared to a PLGA microsphere-based scaffold.

FIG. 15 includes histological photographs of hUCMSCs grown in nonwoven poly-glycolic acid (PGA) scaffolds.

FIG. 16 includes histological photographs of IHC staining for collagen types I and II and aggrecan hUCMSCs grown in nonwoven poly-glycolic acid (PGA) scaffolds.

FIG. 17A includes histological photographs that show immunohistochemical staining for collagen type I and aggrecan after 3 weeks of culture on PGA scaffolds.

FIG. 17B shows hydroxyproline content for hUCMSC and BMSCs after 3 and 6 weeks of culture.

FIG. 18 is a graph that illustrates mineralization after in vitro culture of hUCMSCs on nonwoven poly-glycolic acid (PGA) scaffolds.

FIGS. 19A-19B are photographs that show surgical implantation of a microsphere-based scaffold and tissue growth over the microsphere-based scaffold.

FIGS. 20A-20B show histological results following 6 weeks of microsphere-based scaffold implantation in rabbit knees, and show Saf-O/Fast green staining of the implant/tissue.

FIG. 20C includes histological photographs with Von Kossa staining.

FIG. 20D includes histological photographs with Alizarin Red staining.

FIG. 21 is a graph that shows the growth in cell numbers on blank and gradient microsphere-based scaffolds.

FIG. 22 is a graph that shows the glycosaminoglycan production from cell growth on blank and gradient microsphere-based scaffolds.

FIG. 23A is a graph of a Coulter multisizer size distribution plot of PLG microspheres of different nominal sizes, displaying the monodispersity of the microspheres with discrete peaks (e.g., peaks with % volume less than 0.5 have been omitted for the sake of clarity).

FIG. 23 B includes an image of various shape-specific scaffolds that were produced with PLG microspheres (140 μm) using CO₂ at sub-critical conditions (15 bar for 1 hour at 25° C. followed by depressurization at ˜0.14-0.21 bar/s) utilizing rubber molds of different shapes (e.g., From left to right: cylinder, bilayered cylinder, tube, plus-sign, and star shapes).

FIGS. 24A-24F include scanning electron micrographs of scaffolds fabricated using different types of PLG microspheres at processing conditions for sintering (e.g., CO₂ exposure at 15 bar for 1 hour at 25° C. followed by depressurization at ˜0.14-0.21 bar/s). Sizes of the microspheres used were 240 μm (FIGS. 24A-24B), 175 μm (FIG. 24C), 140 μm (FIGS. 24D-24E), and 140 μm together with 5 μm (FIG. 24F). The morphology of a microsphere following the CO₂ sintering (FIG. 24E) is also displayed.

FIG. 25 is a graph of the modulus of elasticity of the scaffolds prepared using different microspheres sizes.

FIG. 26A-26C includes fluorescence micrographs of live/dead dye-stained porcine chondrocytes seeded on CO₂-sintered microsphere-based scaffolds (175 μm) following a 3 week cell culture period: FIG. 26A) live and dead cells, FIG. 26B) live cells only, and FIG. 26C) dead cells only.

FIG. 26D includes pictures of immunohistochemistry staining for collagen types I and II after 3 weeks of culture on CO₂-sintered microsphere-based scaffolds: HUCMSCs=human umbilical cord mesenchymal stromal cells, CI=collagen type I, CII=collagen type II, and GAG=glycosaminoglycan.

FIG. 27A is a schematic representation of a process of producing a microsphere-based cell-loaded scaffold or thin patch with gaseous CO₂. The process of combining the cells and microparticles in a liquid medium results in a melded thin patch (top), whereas mechanically mixing a loose cell pellet in a minimal liquid volume with the microparticles results in a homogeneously seeded scaffold.

FIGS. 27B-27C include fluorescence micrographs of live/dead dye-stained hUCMSCs that display cell survival following CO₂ sintering of microspheres (120 μm) at sub-critical (gaseous) conditions. FIG. 27B is a thin patch, and FIG. 27C is a macroscopic scaffold.

FIGS. 28A-28D are micrographs showing morphological characteristics of microspheres with various encapsulated nanophase materials.

FIGS. 29A-29C include characteristic SEM micrographs of a scaffold, prepared by sintering the microspheres (90:10 PLGA:CaCO3) using ethanol sintering.

FIG. 29D is a micrograph of a 100 μm thick section of an interior section of a scaffold

FIG. 30A is an image of a gradient scaffold prepared using dye (Rhodamine B)-loaded PLGA microspheres and 90:10 PLGA:CaCO3 microspheres using a 2 hour ethanol soak.

FIG. 30B is a graph that shows the fluorescent gradient which corresponds with the microsphere gradient.

FIG. 31 is a graph that shows the moduli of elasticity of the homogeneous scaffolds prepared using different types of microspheres.

FIGS. 32-32A illustrate embodiments of an engineered particle having a hard core and one or more polymeric shells encapsulating the core.

FIG. 33 illustrates embodiments of shapes that can be formed by sintering the engineered particles.

FIG. 34A includes images of uncoated glass beads.

FIG. 34B includes images of glass beads coated with PLGA.

FIG. 35A shows a phase contrast image of uncoated glass beads.

FIG. 35B shows a phase contrast image of glass beads coated with PLGA.

FIG. 36 shows an engineered particle having a scratch on the surface of the polymer, where the arrow points to the scratch on the surface to visualize the polymeric coating.

FIG. 37A shows a top view and FIG. 37B shows a side view of a body formed from selective laser sintering of the engineered particles so that the polymeric shells meld together at sintered location, and where no sintering leaves loose particles that can be removed to form the holes, which provides the shape of a window frame.

FIGS. 38A and 38B show side views of implants with engineered particles with the polymeric shells melded together with methylene chloride with 3 hours of sintering (FIG. 38A) and 4 hours of sintering (FIG. 38B).

FIG. 39 shows PLGA coated glass beads sintered by sub-critical CO₂.

FIG. 40A shows a scaffold of engineered particles melded together before compression.

FIG. 40B shows a lateral view of the scaffold of FIG. 40A after compression.

FIG. 40C shows cross sectional view after compression.

FIG. 41 shows a graph of data for stress versus strain of a scaffold prepared from PLGA-PCL dual coated particles melded for 3 hours with methylene chloride.

FIGS. 42A and 42B include images of 1 mm engineered particles having the hard core and polymeric shells with live:dead cells at one week, which images are color contrasted to show the cells.

FIG. 43 includes images of 1 mm engineered particles having the hard core and polymeric shells with live:dead cells at 24 hours, which images are color contrasted to show the cells.

FIGS. 44A and 44B include images of 200 micron engineered particles having the hard core and polymeric shells with live:dead cells at one week, which images are color contrasted to show the cells.

FIGS. 45A and 45B include images of 200 micron engineered particles having the hard core and polymeric shells with live:dead cells at 24 hours, which images are color contrasted to show the cells.

FIG. 46A includes an image of 200 micron engineered particles in a sintered scaffold with interstitial spaces between the particles.

FIG. 46B includes an image of 1 mm engineered particles in a sintered scaffold with interstitial spaces between the particles.

FIG. 47 includes a graph that illustrates the average elastic modulus of PCL coated 200 micron glass bead scaffolds compared to with 200 micron PCL microsphere scaffolds.

### DETAILED DESCRIPTION

Spatial and temporal control of bioactive signals in three-dimensional (3-D) tissue engineering scaffolds is greatly desired. Coupled together these attributes may mimic and maintain complex biological signal patterns, such as those observed during axonal regeneration or neovascularization. Seamless polymer tissue engineering scaffolds may provide a route to achieve precise spatial control of signal distribution. A novel microparticle-based scaffold fabrication technique can provide such seamless scaffolds. A method of creating 3-D scaffolds with spatial control over model dyes using uniform Poly(D,L-lactide-co-glycolide) (PLG) microspheres can prepare such seamless scaffolds. Uniform microspheres can be produced using a Precision Particle Fabrication technique. Scaffolds can be assembled by flowing microsphere suspensions into a cylindrical glass mold, and fusing the microspheres to form a continuous, seamless scaffold using ethanol as a melding agent. Morphological and physical characterization of the scaffolds can show microsphere matrices are porous and well connected, and the compressive stiffness of the scaffolds can range from 4.2 to 6.0 MPa. Culturing chondrocytes on the scaffolds can show the compatibility of these substrates with cell attachment and viability. In addition, bi-layered, multi-layered and gradient scaffolds can be fabricated, exhibiting excellent spatial control and resolution. Such novel scaffolds can serve as sustained delivery devices of heterogeneous signals in a continuous and seamless manner, and can be particularly useful in interfacial tissue engineering.

### I. Microsphere-Based Scaffolds

A novel and cost-efficient method has been developed in order to create microsphere-based three-dimensional materials with precise control over their spatial patterns/profiles of biomaterials, porosity and/or bioactive signals, which may be utilized in a variety of applications, such as tissue generation and/or regeneration. Moreover, with a suitable choice of biomaterial, it has been shown that the synthesis and encapsulation process is conducive to cell viability. Specifically, the technique can be used to create gradient scaffolds that can be used in diverse areas of tissue engineering applications, including nerve tissue engineering, study of chemotaxis, directed angiogenesis, spatial regulation of chemokines for modulating immune response, interfacial tissue engineering, and the like.

Microsphere-based three-dimensional tissue engineering scaffolds can contain predefined spatial patterns/profiles of biomaterials, porosity and/or bioactive signals. The process of making the three-dimensional tissue engineering scaffolds successfully produces porous, well-connected matrices, which may be suitable for a variety of tissue engineering applications depending on the selection of suitable biomaterial(s). The process provides excellent spatial resolution over the constituents and can be used to create a variety of gradient profiles, including linear as well as non-linear gradients. The process can be used to create porous, biocompatible and biodegradable scaffolds using microspheres made of, for example, poly(D,L-lactide-co-glycolide) (PLG). Scaffolds with predefined spatial-loaded (encapsulated or surface-immobilized) microspheres can be prepared. Additionally, porosity patterns can be created within a scaffold using microspheres of different sizes.

Scaffolds can be fabricated by flowing microsphere suspensions into a mold of pre-determined shape (to allow fabrication of shape-specific materials) with predefined flow profiles via low dead-volume tubings. The microspheres can be fused to form a continuous material with a melding agent such as ethanol, heat, or pressurized carbon dioxide. The process can utilize commercially available programmable syringe pumps for the generation of various profiles connected. Motor-driven syringe pumps have previously been used for the generation of gel-based (pH) gradients, however, with applications in electrophoresis. These types of pumps can now be used with liquid microsphere compositions to create three-dimensional tissue engineering scaffolds with various characteristics. The method of manufacturing a tissue engineering scaffold with microspheres is a novel way to synthesize the products, with diversified area of application (useful for many applications, including tissue regeneration).

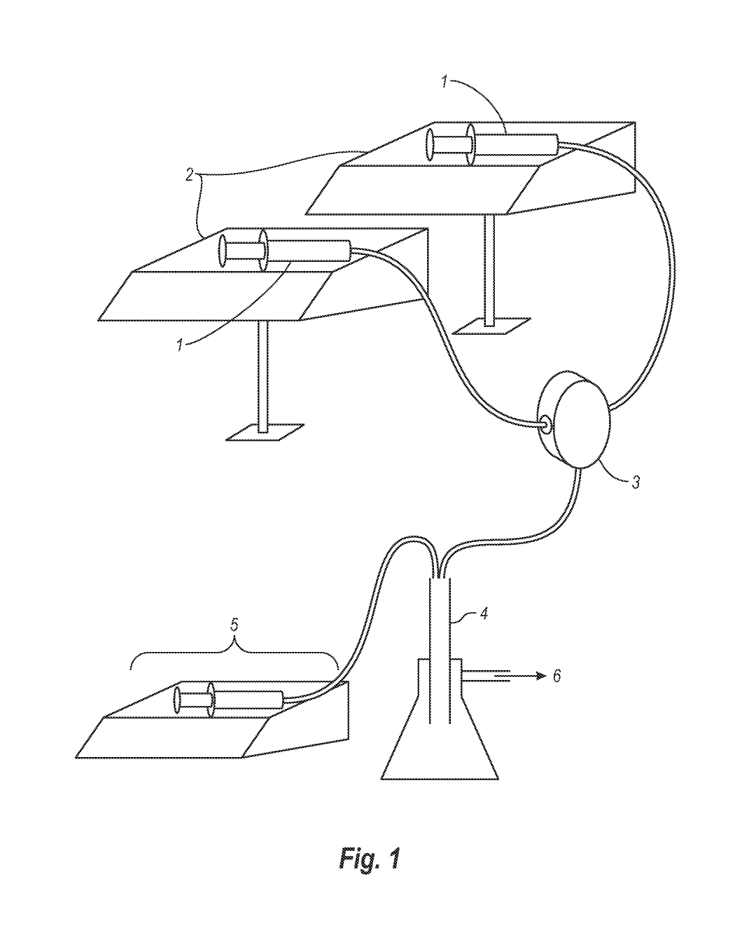

FIG. 1 is a schematic diagram of an embodiment of a scaffold fabrication apparatus in accordance with the present invention. The syringes 1 containing dye-loaded or blank microsphere suspensions in distilled water/PVA solution can be attached to two programmable syringe pumps 2. The suspensions can be pumped in a predefined controlled manner through the attached tubings that join at a coupling 3 into a single tube that can be introduced to a cylindrical glass mold 4. Through the bottom of the mold 4, the distilled water/PVA solution can be constantly filtered, while the microparticles settle in the mold 4. During the entire process, the suspensions in the syringes 1 can be kept homogeneous using external magnetic stirrers (not shown). A constant level of distilled water can be maintained in the mold 4 by an infusion syringe pump 5 and a vacuum pump 6. At the end of the process, distilled water can be completely pulled out using the vacuum pump 6, leaving microparticles stacked in scaffold form in the mold 4.

The present invention can utilize microspheres to create spatial patterning of biological agents that are of special interest to investigators in areas such as nerve tissue engineering, study of chemotaxis, and the like where gradients of surface-immobilized or soluble signaling molecules have been employed. Additionally, the predetermined spatial distribution of the microspheres can be used to modulate the immune and/or inflammatory systems, and controlled spatial and temporal release of anti-inflammatories or chemokines, as desired. In addition, the predetermined spatial distribution of one or more distinct types of microspheres can be used in interfacial tissue engineering to prepare scaffolds with biphasic and gradient distributions of bioactive signals.

In addition, the methods of the present invention can be used to create three-dimensional scaffolds with spatial gradients of soluble growth factor and with controlled temporal release of soluble growth factors. In this regard, the current invention provides a novel method to achieve spatial as well as temporal control over the soluble growth factor (or other bioactive agent) release in three-dimensional scaffolds over time.

In one embodiment, the present invention utilizes growth factor-encapsulated polymeric microspheres (or other biological agent-encapsulated microspheres) as constituents, which are long known to have capability for providing controlled, sustained release. For example, an endoprosthesis can be prepared as a gradient scaffold that is made from growth factor-loaded microspheres, which may serve as novel sustained delivery devices of heterogeneous signals in a continuous and seamless manner for applications in tissue engineering. The process is capable of generating virtually any type of gradient profile. Moreover, the process has the capability to be extended to generate spatial profiles of immobilized growth factors, different polymeric biomaterials, and/or porosity patterns within a scaffold, which are of interest to tissue engineers, in general.

The three-dimensional microsphere structures can be used for the following: osteochondral defect repair (in the presence of growth factors with or without cells) and tissue engineering; axonal regeneration; study of chemotaxis in three-dimensions; directed angiogenesis; regeneration of other interfacial tissues such as muscle-bone, skin layers; temporal and spatial control of release of inflammatory and/or immune system modulators in regenerative medicine applications; and any application requiring a biocompatible, biodegradable material with spatial and temporal control over material composition, bioactive signal release, and porosity.

Microsphere-based scaffolds can provide sustained release of encapsulated growth factors, providing an edge over the soluble protein-containing gel-based scaffolds. To avoid/reduce any change in spatial signal profile, diffusion of the growth factors from the site of release can be regulated by either changing the boundary conditions (e.g., using small-sized wells during in vitro culture, etc.) or by controlling the total amount of growth factor released (balancing the generation and consumption of the growth factors transiently), or controlling the rate of release.

In addition, the microsphere-based scaffolds can be used for establishing in vivo efficacy of gradient scaffolds. For example, preliminary in vivo testing using PLG microspheres encapsulated with osteogenic chondrogenic growth factors for osteochondral defect repair has been performed. As such, the technique is flexible to construct matrices of any polymeric material of the microspheres. Therefore, choosing a microsphere suitable for a particular biomedical/tissue engineering application will eliminate any biomaterial-related concern because any microsphere can be used as appropriate.

For example, uniform PLG microspheres encapsulating fluorescent dyes can create bi-layered, multi-layered, and linear gradient scaffolds. Additionally, the scaffolds made of PLG microspheres were compatible for cell attachment and culture, and had desirable mechanical properties.

In one embodiment, the microspheres can include immobilized surface factors (e.g., RGD adhesion sequences). A distribution of microspheres having immobilized surface factors that produce a gradient of such factors can influence cell migration.

In one embodiment, the present invention includes a novel method of producing a novel composition of matter and methods of using the same. The present invention is an enhancement over other three-dimensional scaffolds by having the ability to create a microsphere-based scaffold material with spatial variation of materials and bioactive signals. Accordingly, the composition of matter has a spatially-controlled microsphere distribution with respect to the three-dimensional scaffold, which can include the spatial control of more than one type of distinct microsphere.

In one embodiment, a method for creating the microsphere-based scaffolds can be performed by flowing two or more different types of distinct microparticles (differing in material, size, encapsulated bioactive signal, and/or tethered surface bioactive signal, etc.) into a mold at desired steady or varying rates. The shape of the final scaffold is determined by the shape of the mold, which can be any desired shape, for example a cylindrical “plug” shape. The microspheres can be melded together by any desired means, including but not limited to, use of a plasticizer, solvent (e.g., ethanol) or gas (e.g., sub-critical carbon dioxide) that functions as a melding agent, high pressure with CO₂, or heat sintering. Based on the controlled release from the microparticles, the design imparts the inherent temporal control of release of bioactive agents from the microsphere in order to provide an overall spatiotemporal (e.g., spatial and temporal) control. In this manner, the microsphere-based scaffolds may be utilized, for example, to provide gradients of stiffness and/or bioactive signals of any desired gradient profile (e.g., linear transition from one side to another).

The microparticle-based scaffolds can have gradients of stiffness, bioactive signals, or the like in a microsphere-based macroporous, interconnected-pore, three-dimensional matrix structure that can be biodegradable. This microsphere-based macroporous, interconnected-pore, three-dimensional matrix structure design can have broad application in several application, for example implantation (into humans or animals), with or without seeded cells, to facilitate tissue generation and/or regeneration or to provide spatiotemporal release of immune mediators.

An increase in the mechanical characteristics of the scaffolds can be achieved by microspheres with a bimodal distribution in the design of the scaffolds, which would provide additional connections between the microspheres and a closer packing.

The microsphere-based scaffold can regulate the temporal presence of growth factors by controlling their release kinetics from their carriers. Spatial control over the release of these bioactive molecules is an aspect that, along with the temporal control, that may provide the possibility of mimicking biological signal patterns, such as those during embryonic development. In the present invention, a novel scaffold fabrication apparatus (FIG. 1) can be used to prepare the microsphere-based scaffolds described herein. The apparatus demonstrated the ability to produce microsphere-based scaffolds with spatial control over molecular composition. The identification of microsphere concentration profiles were obtained using blank or dye-loaded microspheres, which were used as building blocks to fabricate bi-layered, multi-layered and gradient scaffolds.

In comparison to traditional microsphere preparation methods, the methods of the present invention provide the ability to synthesize monodispersed microspheres, which may lead to improved systems to explore the effects of microparticle size on microsphere-based scaffolds. Scaffolds made of uniform microspheres are ideal to study the influence of microparticle size on the degradation patterns and rates within scaffolds. In addition, as observed in the case of colloidal crystal-templated gel-based tissue scaffolds, uniform microspheres can pack closely compared to randomly-sized microspheres, providing better control over the pore-sizes and porosity of the scaffold, and may considerably aid the mechanical integrity of the scaffolds. Moreover, local release of molecules from the microspheres in a bulk scaffold is related to individual microsphere size and polymer properties. Reproducibility and predictability associated with uniform microsphere-based scaffolds may make them suitable for a systematic study of physical and chemical effects in order to achieve control over local release of growth factor within such a scaffold.

Integrated microsphere matrices have been created in the past by employing a heat-sintering technique, which requires heating of microspheres above their glass transition temperatures (Tg). Heat sintering is not suitable for the preparation of bioconductive microsphere-based scaffolds. The inclusion of proteins, polypeptides, nucleic acids, genes viral vectors, small molecules, drugs, antibodies, and growth factors in the microspheres before exposing them to heat may severely affect activity. The sintering temperatures and durations of heat exposure used in some previous studies were 160° C. for 4 h (PLG; 85:15 lactic acid:glycolic acid), 65° C. for 4 h (PLG/bioactive glass), 70° C. for 4 h (poly(D,L-lactide)/poly(ethylene glycol)), and 62° C. for varied times of 4, 24, 48 and 72 h (PLG; 58:42 lactic acid:glycolic acid). Such elevated temperatures for extended durations can lead to reduction in the bioactivity or complete denaturation of encapsulated therapeutic agents, such as nucleic acids and proteins, which has earlier been commented as a concern for the production of matrices made of growth factor-loaded microspheres.

In the present invention, a novel ethanol-melding technique can be used to create interconnected microsphere matrices, which may alleviate concerns of reduced bioactivity from microsphere-based matrices. When compared to heat sintering of PLG microspheres, the process of ethanol melding can result in matrices of lower compressive stiffness. The range of average compressive moduli, reported in a previous study employing heat sintering at 62° C. for 24 h, was 241 to 349 MPa, which was significantly higher than the moduli reported herein. However, the reported porosity of the resulting scaffolds ranged from 32-39%, which were significantly lower than the porosities reported herein. However lower or higher porosities can be obtained by modulating the size of microspheres used as well as the size distribution of microspheres and the duration of melding. As the porosities of such microsphere-based matrices reflect the extent of fusion of the adjacent microspheres within a given matrix, the differences in the porosities may explain the differences observed in the macro-mechanical properties. Increasing ethanol-soak time, changing the microsphere size, or providing microspheres of different sizes may lead to improved mechanical characteristics and, may be used to modulate scaffold porosity.

The effect of ethanol melding on the glass transition temperature (Tg) of the raw PLG can be determined. It was found that the Tg can drop below 37° C., which may affect the mechanical properties of the scaffolds when placed in vivo. To keep the Tg of the scaffolds above the limit of 37° C., the use of PLG with higher molecular weights, crosslinking, different sized microspheres, or PLG with a higher lactic acid to glycolic acid ratios can be used. Also, the microspheres can be doped with polymers or the materials or formed as copolymers that have higher glass transition temperatures.

The present invention provides a manufacturing system and methods for manufacturing microparticle-based gradient scaffolds. The scaffolds can be designed to release opposing gradients of bioactive signals (e.g., biological proteins) at the interface of a biphasic tissue engineering scaffold. The methodology may also be extended to create biphasic scaffolds with more than two growth factors or multi-phasic scaffold with more than one interface. The use of ethanol, acetone, or other solvent or media (e.g., carbon dioxide) as a melding agent can create interconnected microsphere-based matrices, for encapsulated, thermally labile bioactive molecules. For example, growth factor-loaded microspheres may be used to create similar heterogeneous three-dimensional scaffolds to deliver growth factors with pre-defined spatial and temporal release profiles. Also, the scaffolds can be prepared as seamless gradient scaffolds, bi-layered scaffolds, or the like for osteochondral tissue regeneration.

In one embodiment, the microsphere-based scaffolds can be used in osteochondral tissue engineering. As such, the scaffolds can be used to prepare cartilage, bond, or a junction of cartilage and bone by use of heterogeneous signals provided by the use of two or more microspheres having two or more active agents for use in osteochondral tissue engineering. The use of heterogeneous signals can provide mutually inductive signals that are important for the formation of the bone and/or cartilage.

In one embodiment, the microsphere-based scaffolds can be prepared from PLG or PLGA microspheres. However the microspheres can be prepared from substantially any polymer, such as biocompatible, bioerodable, and/or biodegradable polymers. Examples of such biocompatible polymeric materials can include a suitable hydrogel, hydrophilic polymer, hydrophobic polymer biodegradable polymers, bioabsorbable polymers, and monomers thereof. Examples of such polymers can include nylons, poly(alpha-hydroxy esters), polylactic acids, polylactides, poly-L-lactide, poly-DL-lactide, poly-L-lactide-co-DL-lactide, polyglycolic acids, polyglycolide, polylactic-co-glycolic acids, polyglycolide-co-lactide, polyglycolide-co-DL-lactide, polyglycolide-co-L-lactide, polyanhydrides, polyanhydride-co-imides, polyesters, polyorthoesters, polycaprolactones, polyesters, polyanhydrides, polyphosphazenes, poly(phosphoesters), polyester amides, polyester urethanes, polycarbonates, polytrimethylene carbonates, polyglycolide-co-trimethylene carbonates, poly(PBA-carbonates), polyfumarates, polypropylene fumarate, poly(p-dioxanone), polyhydroxyalkanoates, polyamino acids, poly-L-tyrosines, poly(beta-hydroxybutyrate), polyhydroxybutyrate-hydroxyvaleric acids, polyethylenes, polypropylenes, polyaliphatics, polyvinylalcohols, polyvinylacetates, hydrophobic/hydrophilic copolymers, alkylvinylalcohol copolymers, ethylenevinylalcohol copolymers (EVAL), propylenevinylalcohol copolymers, polyvinylpyrrolidone (PVP), poly(L-lysine), poly(lactic acid-co-lysine), poly(lactic acid-graft-lysine), polyanhydrides (such as poly(fatty acid dimer), poly(fumaric acid), poly(sebacic acid), poly(carboxyphenoxy propane), poly(carboxyphenoxy hexane), poly(anhydride-co-imides), poly(amides), poly(iminocarbonates), poly(urethanes), poly(organophasphazenes), poly(phosphates), poly(ethylene vinyl acetate) and other acyl substituted cellulose acetates and derivatives thereof, poly(amino acids), poly(acrylates), polyacetals, poly(cyanoacrylates), poly(styrenes), poly(vinyl chloride), poly(vinyl fluoride), poly(vinyl imidazole), chlorosulfonated polyolefins, polyethylene oxide, combinations thereof, polymers having monomers thereof, or the like. In certain preferred aspects, the nano-particles include hydroxypropyl cellulose (HPC), N-isopropylacrylamide (NIPA), polyethylene glycol, polyvinyl alcohol (PVA), polyethylenimine, chitosan, chitin, dextran sulfate, heparin, chondroitin sulfate, gelatin, etc. and their derivatives, co-polymers, and mixtures thereof. A non-limiting method for making nano-particles is described in U.S. Publication 2003/0138490, which is incorporated by reference.

The methods of making the scaffolds from the microspheres can be changed to include a solvent or solvent system (i.e., media or media system) that is compatible with the particular polymer of the microsphere. That is, the solvent or solvent system can be selected to meld the microspheres together as described herein. Examples of some solvents can include hexane, benzene, toluene, diethyl ether, chloroform, ethyl acetate, 1,4-dioxane, tetrahydrofuran, dichloromethane, acetone, acetonitrile, dimethylformamide, dimethyl sulfoxide, acetic acid, n-butanol, 2-butanol, 3-butanol, t-butyl alcohol, carbon tetrachloride, chlorobenzene, isopropanol, n-propanol, ethanol, methanol, formic acid, water, cyclohexane, 1,2-dichloroethane, diethyl ether, diethylene glycol, diglyme, dimethyl ether, dioxane, ethylene glycol, glycerin, heptane, hexamethylphosphoramide, hesamethylphosphorous triamide, hexane, nitromethane, pentane, petroleum ether, propanol, pyridine, o-xylene, m-xylene, p-xylene, and the like. Carbon dioxide can also be used as a solvent or media to meld the microspheres together. Also, solvents known for particular polymers can be used or combined with the solvents described herein.

The scaffolds can be prepared to contain and release substantially any therapeutic agent, such as release from the shell. Examples of some pharmaceutics agents that be useful in scaffolds for use in a body lumen, such as a blood vessel can include: anti-proliferative/antimitotic agents including natural products such as vinca alkaloids (i.e. vinblastine, vincristine, and vinorelbine), paclitaxel, epidipodophyllotoxins (i.e. etoposide, teniposide), antibiotics (dactinomycin (actinomycin D) daunorubicin, doxorubicin and idarubicin), anthracyclines, mitoxantrone, bleomycins, plicamycin (mithramycin) and mitomycin, enzymes (L-asparaginase which systemically metabolizes L-asparagine and deprives cells which do not have the capacity to synthesize their own asparagine); antiplatelet agents such as G(GP) II b/III a inhibitors and vitronectin receptor antagonists; anti-proliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nirtosoureas (carmustine (BCNU) and analogs, streptozocin), trazenes-dacarbazinine (DTIC); anti-proliferative/antimitotic antimetabolites such as folic acid analogs (methotrexate), pyrimidine analogs (fluorouracil, floxuridine, and cytarabine), purine analogs and related inhibitors (mercaptopurine, thioguanine, pentostatin and 2-chlorodeoxyadenosine {cladribine}); platinum coordination complexes (cisplatin, carboplatin), procarbazine, hydroxyurea, mitotane, aminoglutethimide; hormones (i.e. estrogen); anti-coagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory; antisecretory (breveldin); anti-inflammatory: such as adrenocortical steroids (cortisol, cortisone, fludrocortisone, prednisone, prednisolone, 6α-methylprednisolone, triamcinolone, betamethasone, and dexamethasone), non-steroidal agents (salicylic acid derivatives e.g., aspirin; para-aminophenol derivatives i.e. acetaminophen; indole and indene acetic acids (indomethacin, sulindac, and etodalac), heteroaryl acetic acids (tolmetin, diclofenac, and ketorolac), arylpropionic acids (ibuprofen and derivatives), anthranilic acids (mefenamic acid, and meclofenamic acid), enolic acids (piroxicam, tenoxicam, phenylbutazone, and oxyphenthatrazone), nabumetone, gold compounds (auranofin, aurothioglucose, gold sodium thiomalate); immunosuppressives: (cyclosporine, tacrolimus (FK-506), sirolimus (rapamycin), everolimus, azathioprine, mycophenolate mofetil); angiogenic agents: vascular endothelial growth factor (VEGF), fibroblast growth factor (FGF); angiotensin receptor blockers; nitric oxide donors; antisense oligionucleotides and combinations thereof; cell cycle inhibitors, mTOR inhibitors, and growth factor receptor signal transduction kinase inhibitors; retenoids; cyclin/CDK inhibitors; HMG co-enzyme reductase inhibitors (statins); and protease inhibitors; β 2 agonists (e.g. salbutamol, terbutaline, clenbuterol, salmeterol, formoterol); steroids such glycocorticosteroids, preferably anti-inflammatory drugs (e.g. Ciclesonide, Mometasone, Flunisolide, Triamcinolone, Beclomethasone, Budesonide, Fluticasone); anticholinergic drugs (e.g. ipratropium, tiotropium, oxitropium); leukotriene antagonists (e.g. zafirlukast, montelukast, pranlukast); xantines (e.g. aminophylline, theobromine, theophylline); Mast cell stabilizers (e.g. cromoglicate, nedocromil); inhibitors of leukotriene synthesis (e.g. azelastina, oxatomide ketotifen); mucolytics (e.g. N-acetylcysteine, carbocysteine); antibiotics, (e.g. Aminoglycosides such as, amikacin, gentamicin, kanamycin, neomycin, netilmicin streptomycin, tobramycin; Carbacephem such as loracarbef, Carbapenems such as ertapenem, imipenem/cilastatin meropenem; Cephalosporins-first generation—such as cefadroxil, cefaxolin, cephalexin; Cephalosporins-second generation—such as cefaclor, cefamandole, defoxitin, cefproxil, cefuroxime; Cephalosporins-third generation-cefixime, cefdinir, ceftaxidime, defotaxime, cefpodoxime, ceftriaxone; Cephalosporins—fourth generation—such as maxipime; Glycopeptides such as vancomycin, teicoplanin; Macrolides such as azithromycin, clarithromycin, Dirithromycin, Erythromycin, troleandomycin; Monobactam such as aztreonam; Penicillins such as Amoxicillin, Ampicillin, Azlocillin, Carbenicillin, Cloxacillin, Dicloxacillin, Flucloxacillin, Mezlocillin, Nafcillin, Penicillin, Piperacillin, Ticarcillin; Polypeptides such as bacitracin, colistin, polymyxin B; Quinolones such as Ciprofloxacin, Enoxacin, Gatifloxacin, Levofloxacin, Lomefloxacin, Moxifloxacin, Norfloxacin, Ofloxacin, Trovafloxacin; Sulfonamides such as Mafenide, Prontosil, Sulfacetamide, Sulfamethizole, Sulfanilimide, Sulfasalazine, Sulfisoxazole, Trimethoprim, Trimethoprim-Sulfamethoxazole Co-trimoxazole (TMP-SMX); Tetracyclines such as Demeclocycline, Doxycycline, Minocycline, Oxytetracycline, Tetracycline; Others such as Chloramphenicol, Clindamycin, Ethambutol, Fosfomycin, Furazolidone, Isoniazid, Linezolid, Metronidazole, Nitrofurantoin, Pyrazinamide, Quinupristin/Dalfopristin, Rifampin, Spectinomycin); pain relievers in general such as analgesic and antiinflammatory drugs, including steroids (e.g. hydrocortisone, cortisone acetate, prednisone, prednisolone, methylpredniso lone, dexamethasone, betamethasone, triamcinolone, beclometasone, fludrocortisone acetate, deoxycorticosterone acetate, aldosterone); and non-steroid antiinflammatory drugs (e.g. Salicylates such as aspirin, amoxiprin, benorilate, coline magnesium salicylate, diflunisal, faislamine, methyl salicylate, salicyl salicylate); Arylalkanoic acids such as diclofenac, aceclofenac, acematicin, etodolac, indometacin, ketorolac, nabumetone, sulindac tolmetin; 2-Arylpropionic acids (profens) such as ibuprofen, carprofen, fenbufen, fenoprofen, flurbiprofen, ketoprofen, loxoprofen, naproxen, tiaprofenic acid; N-arylanthranilic acids (fenamic acids) such as mefenamic acid, meclofenamic acid, tolfenamic acid; Pyrazolidine derivatives such as phenylbutazone, azapropazone, metamizole, oxyphenbutazone; Oxicams such as piroxicam, meloxicam, tenoxicam; Coxib such as celecoxib, etoricoxib, lumiracoxib, parecoxib, rofecoxib (withdrawn from market), valdecoxib (withdrawn from market); Sulphonanilides such as nimesulide; others such as licofelone, omega-3 fatty acids; cardiovascular drugs such as glycosides (e.g. strophantin, digoxin, digitoxin, proscillaridine A); respiratory drugs; antiasthma agents; bronchodilators (adrenergics: albuterol, bitolterol, epinephrine, fenoterol, formoterol, isoetharine, isoproterenol, metaproterenol, pirbuterol, procaterol, salmeterol, terbutaline); anticancer agents (e.g. cyclophosphamide, doxorubicine, vincristine, methotrexate); alkaloids (i.e. ergot alkaloids) or triptans such as sumatriptan, rizatriptan, naratriptan, zolmitriptan, eletriptan and almotriptan, than can be used against migraine; drugs (i.e. sulfonylurea) used against diabetes and related dysfunctions (e.g. metformin, chlorpropamide, glibenclamide, glicliazide, glimepiride, tolazamide, acarbose, pioglitazone, nateglinide, sitagliptin); sedative and hypnotic drugs (e.g. Barbiturates such as secobarbital, pentobarbital, amobarbital; uncategorized sedatives such as eszopiclone, ramelteon, methaqualone, ethchlorvynol, chloral hydrate, meprobamate, glutethimide, methyprylon); psychic energizers; appetite inhibitors (e.g. amphetamine); antiarthritis drugs (NSAIDs); antimalaria drugs (e.g. quinine, quinidine, mefloquine, halofantrine, primaquine, cloroquine, amodiaquine); antiepileptic drugs and anticonvulsant drugs such as Barbiturates, (e.g. Barbexaclone, Metharbital, Methylphenobarbital, Phenobarbital, Primidone), Succinimides (e.g. Ethosuximide, Mesuximide, Phensuximide), Benzodiazepines, Carboxamides (e.g. Carbamazepine, Oxcarbazepine, Rufinamide) Fatty acid derivatives (e.g. Valpromide, Valnoctamide); Carboxilyc acids (e.g. Valproic acid, Tiagabine); Gaba analogs (e.g. Gabapentin, Pregabalin, Progabide, Vigabatrin); Topiramate, Ureas (e.g. Phenacemide, Pheneturide), Carbamates (e.g. emylcamate Felbamate, Meprobamate); Pyrrolidines (e.g. Levetiracetam Nefiracetam, Seletracetam); Sulfa drugs (e.g. Acetazolamide, Ethoxzolamide, Sultiame, Zonisamide) Beclamide; Paraldehyde, Potassium bromide; antithrombotic drugs such as Vitamin K antagonist (e.g. Acenocoumarol, Dicumarol, Phenprocoumon, Phenindione, Warfarin); Platelet aggregation inhibitors (e.g. antithrombin III, Bemiparin, Deltaparin, Danaparoid, Enoxaparin, Heparin, Nadroparin, Pamaparin, Reviparin, Tinzaparin); Other platelet aggregation inhibitors (e.g. Abciximab, Acetylsalicylic acid, Aloxiprin, Ditazole, Clopidogrel, Dipyridamole, Epoprostenol, Eptifibatide, Indobufen, Prasugrel, Ticlopidine, Tirofiban, Treprostinil, Trifusal); Enzymes (e.g. Alteplase, Ancrod, Anistreplase, Fibrinolysin, Streptokinase, Tenecteplase, Urokinase); Direct thrombin inhibitors (e.g. Argatroban, Bivalirudin. Lepirudin, Melagatran, Ximelagratan); other antithrombotics (e.g. Dabigatran, Defibrotide, Dermatan sulfate, Fondaparinux, Rivaroxaban); antihypertensive drugs such as Diuretics (e.g. Bumetanide, Furosemide, Torsemide, Chlortalidone, Hydroclorothiazide, Chlorothiazide, Indapamide, metolaxone, Amiloride, Triamterene); Antiadrenergics (e.g. atenolol, metoprolol, oxprenolol, pindolol, propranolol, doxazosin, prazosin, teraxosin, labetalol); Calcium channel blockers (e.g. Amlodipine, felodipine, dsradipine, nifedipine, nimodipine, diltiazem, verapamil); Ace inhibitors (e.g. captopril, enalapril, fosinopril, lisinopril, perindopril, quinapril, ramipril, benzapril); Angiotensin II receptor antagonists (e.g. candesartan, irbesartan, losartan, telmisartan, valsartan); Aldosterone antagonist such as spironolactone; centrally acting adrenergic drugs (e.g. clonidine, guanabenz, methyldopa); antiarrhythmic drug of Class I that interfere with the sodium channel (e.g. quinidine, procainamide, disodyramide, lidocaine, mexiletine, tocamide, phenyloin, encamide, flecamide, moricizine, propafenone), Class II that are beta blockers (e.g. esmolol, propranolol, metoprolol); Class III that affect potassium efflux (e.g. amiodarone, azimilide, bretylium, clorilium, dofetilide, tedisamil, ibutilide, sematilide, sotalol); Class IV that affect the AV node (e.g. verapamil, diltiazem); Class V unknown mechanisms (e.g. adenoide, digoxin); antioxidant drugs such as Vitamin A, vitamin C, vitamin E, Coenzime Q10, melanonin, carotenoid terpenoids, non carotenoid terpenoids, flavonoid polyphenolic; antidepressants (e.g. mirtazapine, trazodone); antipsychotic drugs (e.g. fluphenazine, haloperidol, thiotixene, trifluoroperazine, loxapine, perphenazine, clozapine, quetiapine, risperidone, olanzapine); anxyolitics (Benzodiazepines such as diazepam, clonazepam, alprazolam, temazepam, chlordiazepoxide, flunitrazepam, lorazepam, clorazepam; Imidaxopyridines such as zolpidem, alpidem; Pyrazolopyrimidines such as zaleplon); antiemetic drugs such as Serotonine receptor antagonists (dolasetron, granisetron, ondansetron), dopamine antagonists (domperidone, droperidol, haloperidol, chlorpromazine, promethazine, metoclopramide) antihistamines (cyclizine, diphenydramine, dimenhydrinate, meclizine, promethazine, hydroxyzine); antiinfectives; antihistamines (e.g. mepyramine, antazoline, diphenihydramine, carbinoxamine, doxylamine, clemastine, dimethydrinate, cyclizine, chlorcyclizine, hydroxyzine, meclizine, promethazine, cyprotheptadine, azatidine, ketotifen, acrivastina, loratadine, terfenadine, cetrizidinem, azelastine, levocabastine, olopatadine, levocetrizine, desloratadine, fexofenadine, cromoglicate nedocromil, thiperamide, impromidine); antifungus (e.g. Nystatin, amphotericin B, natamycin, rimocidin, filipin, pimaricin, miconazole, ketoconazole, clotrimazole, econazole, mebendazole, bifonazole, oxiconazole, sertaconazole, sulconazole, tiaconazole, fluconazole, itraconazole, posaconazole, voriconazole, terbinafine, amorolfine, butenafine, anidulafungin, caspofungin, flucytosine, griseofulvin, fluocinonide) and antiviral drugs such as Anti-herpesvirus agents (e.g. Aciclovir, Cidofovir, Docosanol, Famciclovir, Fomivirsen, Foscarnet, Ganciclovir, Idoxuridine, Penciclovir, Trifluridine, Tromantadine, Valaciclovir, Valganciclovir, Vidarabine); Anti-influenza agents (Amantadine, Oseltamivir, Peramivir, Rimantadine, Zanamivir); Antiretroviral drugs (abacavir, didanosine, emtricitabine, lamivudine, stavudine, tenofovir, zalcitabine, zidovudine, adeforvir, tenofovir, efavirenz, delavirdine, nevirapine, amprenavir, atazanavir, darunavir, fosamprenavir, indinavir, lopinavir, nelfinavir, ritonavir, saquinavir, tipranavir); other antiviral agents (Enfuvirtide, Fomivirsen, Imiquimod, Inosine, Interferon, Podophyllotoxin, Ribavirin, Viramidine); drugs against neurological dysfunctions such as Parkinson's disease (e.g. dopamine agonists, L-dopa, Carbidopa, benzerazide, bromocriptine, pergolide, pramipexole, ropinipole, apomorphine, lisuride); drugs for the treatment of alcoholism (e.g. antabuse, naltrexone, vivitrol), and other addiction forms; vasodilators for the treatment of erectile dysfunction (e.g. Sildenafil, vardenafil, tadalafil), muscle relaxants (e.g. benzodiazepines, methocarbamol, baclofen, carisoprodol, chlorzoxazone, cyclobenzaprine, dantrolene, metaxalone, orphenadrine, tizanidine); muscle contractors; opioids; stimulating drugs (e.g. amphetamine, cocaina, caffeine, nicotine); tranquilizers; antibiotics such as macrolides; aminoglycosides; fluoroquinolones and β-lactames; vaccines; cytokines; growth factors; hormones including birth-control drugs; sympathomimetic drugs (e.g. amphetamine, benzylpiperazine, cathinone, chlorphentermine, clobenzolex, cocaine, cyclopentamine, ephedrine, fenfluramine, methylone, methylphenidate, Pemoline, phendimetrazine, phentermine, phenylephrine, propylhexedrine, pseudoephedrine, sibutramine, symephrine); diuretics; lipid regulator agents; antiandrogen agents (e.g. bicalutamide, cyproterone, flutamide, nilutamide); antiparasitics; blood thinners (e.g. warfarin); neoplastic drugs; antineoplastic drugs (e.g. chlorambucil, chloromethine, cyclophosphamide, melphalan, carmustine, fotemustine, lomustine, carboplatin, busulfan, dacarbazine, procarbazine, thioTEPA, uramustine, mechloretamine, methotrexate, cladribine, clofarabine, fludarabine, mercaptopurine, fluorouracil, vinblastine, vincristine, daunorubicin, epirubicin, bleomycin, hydroxyurea, alemtuzumar, cetuximab, aminolevulinic acid, altretamine, amsacrine, anagrelide, pentostatin, tretinoin); hypoglicaemics; nutritive and integrator agents; growth integrators; antienteric drugs; vaccines; antibodies; diagnosis and radio-opaque agents; or mixtures of the above mentioned drugs (e.g. combinations for the treatment of asthma containing steroids and β-agonists); or any other biologically active agent such as nucleic acids, DNA, RNA, siRNA, polypeptides, antibodies, and the like. Growth factors and adhesion peptides can be useful for tissue development within a subject and can be included in the microspheres.

The microsphere-based scaffold can be prepared into substantially any shape by preparing a mold to have the desired shape. For example, the microsphere-based scaffold can be prepared into the shapes of rods, plates, spheres, wrappings, patches, plugs, depots, sheets, cubes, blocks, bones, bone portions, cartilage, cartilage portions, implants, orthopedic implants, orthopedic screws, orthopedic rods, orthopedic plates, and the like. Also, the microsphere-based scaffolds can be prepared into shapes to help facilitate the transitions between tissues, such as between bone to tendon, bone to cartilage, tendon to muscle, dentin to enamel, skin layers, disparate layers, and the like. The microsphere-based scaffolds can also be shaped as bandages, plugs, or the like for wound healing.

In one embodiment, the mean theoretical porosities of the scaffolds can have a range between about 10 to about 95, more preferably about 40 to about 40, and most preferably from about 45 to about 50. An example of porosity is about 44.9% to about 49%.

The scaffolds can have pore sizes ranging from about 200 um to about 1650 um. For cartilage tissue engineering, the pore sizes can be from about 70 um to about 120 um. In bone tissue engineering, pore sizes as small as about 50 um or larger can be used. Although optimal pore sizes can be within a range of about 100 um to about 600 um.

In one embodiment, the mean particle size of the microspheres used to prepare the scaffolds can have a range between about less than 1 um to about greater than 1 mm, more preferably about 100 um (e.g., um is microns) to about 300 um, and most preferably from about 180 to about 240 um. An example of particle size is about 180 μm to about 220 μm.

In one embodiment, the average moduli of elasticity of the scaffolds can have a range between about 6 kPa to about 40 MPa, more preferably about 200 kPa to about 8 MPa, and most preferably from about 1 MPa to about 4 MPa. Examples of elasticity can be about 4.2 MPa to about 6.0 MPa or about 5 MPa to about 12 MPa.

In one embodiment, the microspheres and/or microsphere-based scaffolds can be processed with nanophase CaCO3 to produce a nanophase within the microsphere that hardens the microspheres and resulting microsphere-based scaffolds. Nanophase CaCO3 has been found to improve stiffness of microspheres by 2-8 times. FIG. 8 is a graph that shows the elastic modulus at high strains of microspheres of 100 um, 175 um, and 500 um. Accordingly, the stiffness of the microspheres can be modulated to obtain a stiffness of the scaffold to have a range between about 6 kPa to about 40 MPa, more preferably about 200 kPa to about 8 MPa, and most preferably from about 1 MPa to about 4 MPa. Also, the amount of CaCO3 (or any nanophase material) can have a range between about 0 to about 50% by weight, more preferably about 5% to about 30%, and most preferably from about 5% to about 20%. Numerous other materials can be used as a nanophase material to be included in the microspheres, including but not limited to titanium oxide or hydroxyapatite.

In one embodiment, the glass transition temperature of the scaffolds can have a range between about 30° C. to about 65° C., more preferably about 32° C. to about 50° C., and most preferably from about 34° C. to about 42° C. Examples of glass transition temperature can be about 36° C. to about 38° C.

In one embodiment, the microsphere-based scaffold can be prepared to have a mechanical property gradient. For example, one side can be stiffer and the other side can be softer. This can be accomplished by using microspheres with different mechanical properties. Gradients in mechanical properties in native tissues often exist within and between the tissues, which help in avoiding stress concentrations, such as cartilage, human crystalline lens, and the dentin-enamel junction. Studies have recently confirmed that cells sense and respond to the stiffness of their surrounding environment, influencing lineage commitment as well as migration and proliferation. Mesenchymal stem cells can be directed toward neurogenic (soft matrices), myogenic (intermediate stiffness), or osteogenic (high stiffness) lineages based on the stiffness of the surrounding matrix. Thus, a 3D stiffness gradient can be useful, and can be tailored to match bone and cartilage moduli. As such, an appropriate range can be provide for a scaffold construct with mechanical integrity sufficient for gradual increase in weight bearing (e.g., from gentle walking eventually up to active movement) as the construct in the animal or patient remodels and ultimately matches the native tissue. For example, a range of moduli in osteochondral constructs can be about 10 MPa for cartilage and about 50 MPa for bone.

In one embodiment, the microsphere-based scaffold can be prepared with a sub-critical carbon dioxide (CO₂) technique. Sub-critical CO₂ is not toxic and can be used in place of organic solvents. Accordingly, poly(lactic-co-glycolic acid) microspheres of three different sizes (e.g., 100 um, 175, um, and 500 um) can be prepared. The microsphere-based scaffolds can be prepared by melding the microspheres together in a high pressure chamber at sub-critical pressures of CO₂. For example, sub-critical pressures can be 165 psi, 190 psi, and 220 psi, or any range therebetween. However, higher pressures can be used for larger microspheres, and lower pressures for smaller microspheres.

Accordingly, shape-specific, macroporous tissue engineering scaffolds can be fabricated and homogeneously seeded with cells in a single step. This method brings together CO₂ polymer processing and microparticle-based scaffolds in a manner that allows each to solve the key limitation of the other. Specifically, microparticle-based scaffolds prepared from conventional microsphere sintering methods (e.g., heat, solvents) are not cytocompatible. That is, the microspheres cannot be melded together in the presence of live cells without killing the cells. However, it has now been found that cells can be included with the microspheres during the melding process when melded with CO₂. Accordingly, it has been shown that cell viability can be sustained with sub-critical (i.e., gaseous) CO₂ sintering of microspheres in the presence of cells at near-ambient temperatures. The scaffolds prepared with CO₂ also retain the pore structures described herein. On the other hand, pore interconnectivity has eluded supercritical CO₂ foaming approaches. The fused poly(lactide-co-glycolide) microsphere scaffolds can be seeded with any type of cell, such as human umbilical cord mesenchymal stromal cells. Accordingly, the scaffolds can be used for cartilage regeneration, or any tissue engineering application such as those described herein.

In one embodiment, the microsphere scaffolds can be prepared with a liquid medium in the presence of CO₂ in order to produce thin sheet of melded microspheres. The liquid medium can retain the individual microspheres internally, and only the external surface microspheres are melded together to provide the thin sheet. Also, cells can be loaded in the thin sheet of microspheres with the CO₂ and liquid medium process. The thin sheets of melded microspheres, such as the cell-loaded sheets, can be used as patches in skin tissue engineering and wound healing applications.

Gaseous CO₂ sintering can be used to fabricate cell-seeded, microsphere-based, shape-specific constructs in a single step. These constructs can retain the numerous advantages of microsphere-based scaffolds such as spatiotemporal control for creating 3D signal and stiffness gradients for interfacial tissue engineering within a single scaffold. The resulting scaffolds were porous, exhibited moduli similar to the native cartilaginous tissues, and displayed support for chondrogenesis and cartilage-like tissue growth. The process of sub-critical CO₂ sintering is also amenable to produce cell-containing matrices under relatively mild conditions. The ability to create cell-loaded scaffolds and patches may have important implications for cartilage and skin tissue engineering, respectively, where growth factor-encapsulated microspheres can be used to design cell-loaded controlled release vehicles in a single-step as a regenerative protocol.

In one embodiment, the present invention may be used in connection with a diverse type of eukaryotic host cells from a diverse set of species of the plant and animal kingdoms. Preferably, the host cells are from mammalian species including cells from humans, other primates, horses, pigs, and mice. For example, cells can be stem cells of any kind (e.g., umbilical cord or placenta derived, dental pulp derived, marrow-derived, adipose derived, induced stem cells, or cells of embryonic or amniotic origin), PER.C6 cells, HT-29 cells, LNCaP-FGC cells A549 cells, MDA-MB453 cells, HepG2 cells, THP-1 cells, miMCD-3 cells, HEK 293 cells, HeLaS3 cells, MCF7 cells, Cos-7 cells, CHO cells and CHO derivatives, CHO-K1 cells, BxPC-3 cells, DU145 cells, Jurkat cells, PC-3 cells, Capan-1 cells, HuVEC cells, HuASMC cells, HKB-11 human differentiated stem cells such as osteoblasts and adipocytes from hMSC; human adherent cells such as SH-SY5Y, IMR32, LANS, HeLa, MCF10A, 293T, and SK-BR3; primary cells such as HUVEC, HUASMC, and hMSC; and other species such as 3T3 NIH, 3T3 L1, ES-D3, C2C12, H9c2 and the like. Additionally, any species of plant may be used. Any of these cells can be included with the microspheres when using the carbon dioxide process (e.g., single step) for preparing the scaffolds.

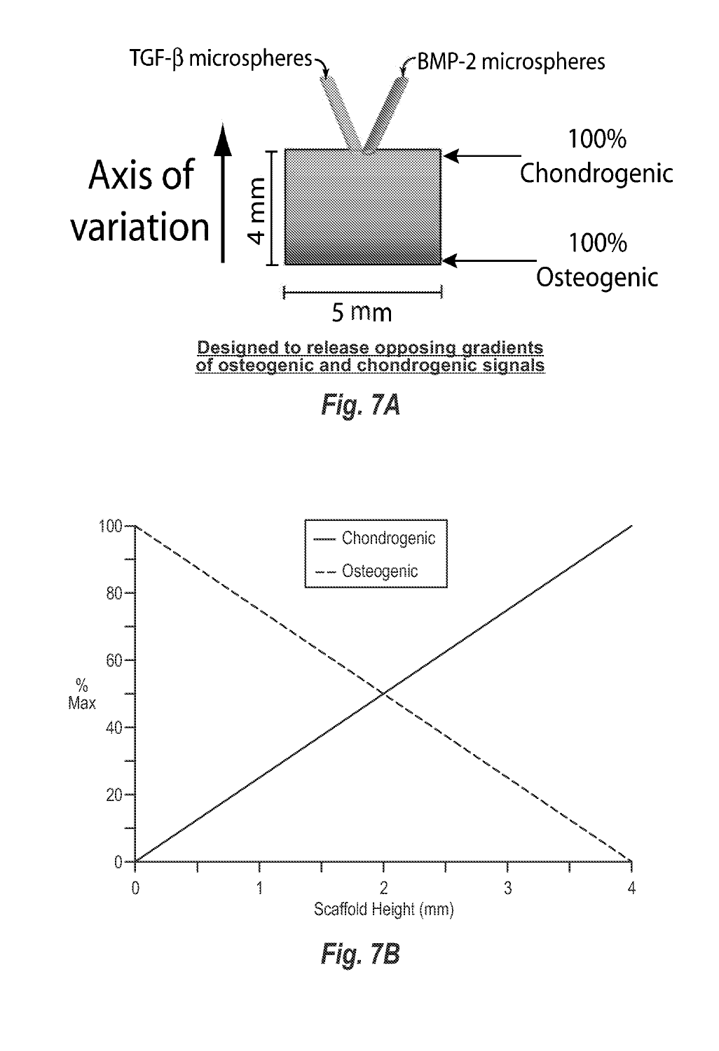

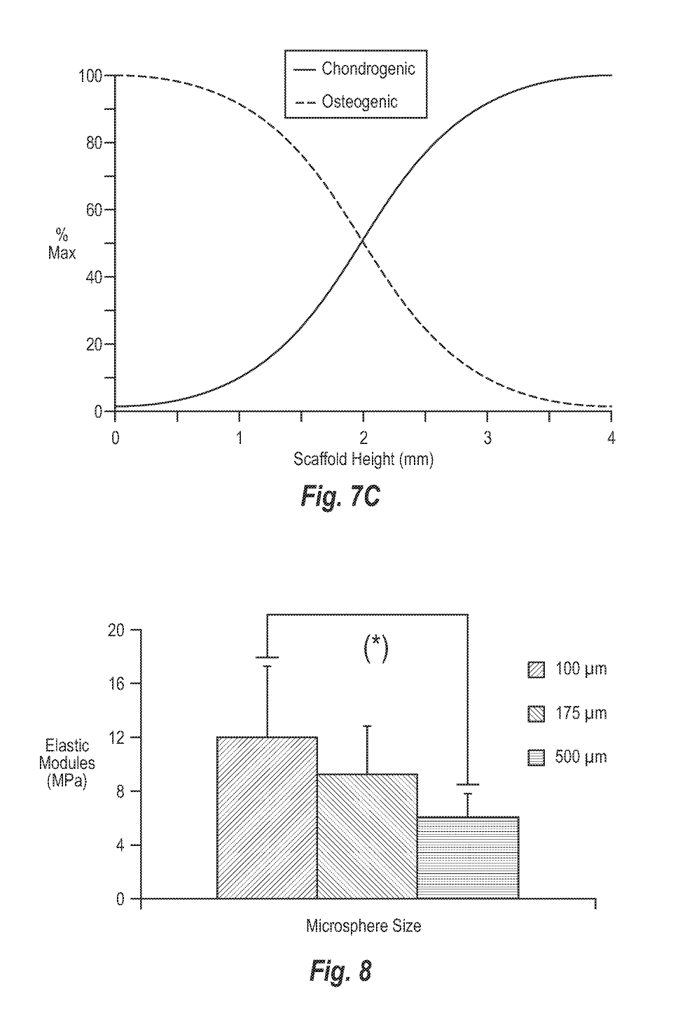

In one embodiment, the microsphere-based scaffold can be prepared to have a gradient of TGF-beta microspheres and another gradient of BMP-2 microspheres, such as is shown in FIG. 7A. As shown, the top of the scaffold is substantially 100% chondrogenic and the bottom of the scaffold is 100% osteogenic. FIG. 7B shows the concentration profile of chondrogenic and osteogenic signals. FIG. 7C shows another concentration profile that can be achieved. Also, the microspheres can use transforming growth factor-beta1 (TGF-β₁) and TGF-β₃ as competing chondrogenic factors, and BMP-2 as the osteogenic factor. In vitro rBMSC chondrogenic differentiation was well demonstrated with the stimulation of TGF-β₁ and/or dexamethasone in cell aggregates and cell pellets. Like TGF-β₁, TGF-β₃ is another known chondrogenic factor, which has been used to promote chondrogenic differentiation of BMSCs in many tissue engineering investigations. TGF-β concentrations in vitro have been commonly prescribed between 5 and 20 ng/mL with different adult stem cells. The concentration of TGF-β₁ used in vivo, however, varies greatly (0.8 to 100 ng per scaffold, ˜4 to 30 ng/mg of carrier). BMP-2 has been extensively applied as an osteogenic factor in bone tissue engineering investigations. Concentrations of BMPs applied in vivo for bone defect repair were usually higher (˜1.6 to 5 μg per mg of polymer) when compared to concentrations of TGF-β₁. Rather than culture disparate cartilaginous and osseous components together in hopes of eventual integration, the microsphere-based scaffolds can provide signal gradients can directly lead to a continuous and seamless transition from cartilage to bone. Also, a signal or combination of signals (e.g., biological molecules) for cartilage to differentiate into its three layers (superficial, middle, deep) can be used in a signal gradient of chondrogenic factors and of bone-generated factors.

In one embodiment, the microsphere-based scaffold can be used in an aerospace application. For example, in applications where a transition from ceramic heat resistance to metallic mechanical integrity is required.

In one embodiment, the microsphere-based scaffold can be used for nerve regeneration. For example, nerve regeneration and axon guidance are heavily influenced by stiffness gradients and signal gradients, which can be provided by these scaffolds.

In one embodiment, the microsphere-based scaffold can be used for craniofacial and orthopedic applications. For example, any instance where there is an interface and thus a graded transition from one tissue type to another (e.g., ligament to bone, tendon to muscle, cartilage to bone, dentin to enamel, etc.), the scaffolds would be well-suited for a seamless transition from one tissue type to another.

In one embodiment, the microsphere-based scaffold can be used as an integrated osteochondral plug. Orthopedic surgeons can implant such a plug in a minimally invasive manner (arthroscope), with or without marrow or umbilical cord cells, to accelerate healing and allow osteoarthritis and impact-injury patients to return to load-bearing activities sooner. Conventional biodegradable plugs currently used have no bioactive signals to accelerate regeneration and do not account for the contrasting mechanical demands of the cartilage and underlying bone. More importantly, the microsphere-based scaffold technology is not limited to osteochondral applications, and can be used in any application where a gradient or integrated interface is desired, such as nerve regeneration, the ligament/bone interface, and the like.

In one embodiment, the microsphere-based scaffold can be prepared with microspheres that include a core and one or more shells. Microspheres with core/shell configurations can be prepared by standard techniques. The core/shell configuration can allow for customized bioactive agent release profiles. For example, the shell can be configured to have one release rate and the core can have a second release rate. Also, the core can have a different bioactive agent compared to the shell. When multiple shells are used, the different shells can have different release rates and/or different bioactive agents.

In one embodiment, the microsphere scaffolds can be prepared as a thin sheet. It has been found that when a medium is used in the carbon dioxide melding process, the liquid can keep the microspheres from melding in the bulk, but the surface microspheres meld in the presence of CO₂. The presence of a liquid (e.g., cells suspended in medium) provides a different result compared to when the liquid volume is minimized (e.g., cell pellet combined with microparticles). With the liquid, the CO₂ can only effectively penetrates the top layer. This results in only the top layer being sintered, resulting in a thin, pliable, cell-seeded polymer. However, cells do not need to be included in the medium, and the resulting thin sheet scaffold can be cell-free. In contrast, the other route provides a heterogeneously seeded construct comparable to the ethanol (or otherwise)-sintered constructs, with the key difference being that the ethanol (or otherwise)-sintered constructs would need to be seeded with cells after the scaffold fabrication, as a separate step. For example, the thin patch can be ideal for skin regeneration (e.g., burn victims). Sub-critical or gaseous CO₂ exposure (e.g., pressure about 15-20 bar) can be used to produce regular or intricate-shaped scaffolds.

When polymers are in the presence of CO₂ as a liquid (e.g., high pressure) or in a supercritical state, a foamed scaffold with non-interconnected pores is formed. However, when microspheres are melded with CO₂ as described herein, pore interconnectivity is obtained as shown and described. Moreover, the CO₂ at mild pressures is a gaseous sub-critical CO₂, which results in milder, and thus likely more cell-compatible conditions. Thus, microsphere sintering can now take place in the presence of cells, made possible with gaseous sub-critical CO₂ in mild conditions.

### II. Experimental Section

### 1. Materials

Poly(D,L-lactide-co-glycolide) copolymer (50:50 lactic acid:glycolic acid; intrinsic viscosity=0.41 dL/g corresponding to Mw ˜50,000 Da) was purchased from Birmingham Polymers (Pelham, Ala.). Poly(vinyl alcohol) (PVA; 88% hydrolyzed, 25,000 Da) was obtained from Polysciences, Inc. (Warrington, Pa.). Rhodamine B base and fluorescein were obtained from Sigma (St. Louis, Mo.). Dichloromethane (DCM; HPLC grade) was obtained from Fisher Scientific (Pittsburgh, Pa.). Ethanol (Absolute—200 Proof) was obtained in house.

### 2. Microsphere Preparation

Uniform PLG microspheres were prepared using technology as previously described using the Precision Particle Fabrication method (Berkland C, Kim K, Pack D W. Fabrication of PLG microspheres with precisely controlled and monodisperse size distributions. J Control Release 2001 May 18; 73(1):59-74). Briefly, PLG dissolved in DCM (30% w/v) was sprayed through a small-gauge needle. The polymer stream was acoustically excited using an ultrasonic transducer (Branson Ultrasonics, Danbury, Conn.) controlled by a waveform generator (model 33220A, Agilent Technologies, Santa Clara, Calif.), resulting in regular jet instabilities that produced uniform polymer droplets. An annular carrier stream (˜0.5% PVA w/v in distilled water) surrounding the droplets was produced using a nozzle coaxial to the needle. The emanated polymer/carrier streams flowed into a beaker containing approximately 1,000 mL of 0.5% PVA. To extract the solvent, incipient polymer droplets were stirred for 3-4 hours. Subsequently, the hardened microspheres were filtered and rinsed with distilled water (˜1 L) to remove residual PVA. Finally, microspheres were lyophilized (Freezone, Labconco benchtop model) for 2 days and stored at −20° C. under desiccant. In a similar manner, fluorescent dye-loaded microspheres were prepared for concentration profile assessment (discussed herein) by using PLG solution (˜30% w/v in DCM) co-dissolved with rhodamine B or fluorescein.

The microspheres having a uniform diameter were characterized for their size and morphology. Four sets of microparticles were produced; a) blank-220 μm, b) blank-160 μm, c) rhodamine B-loaded (10% w/w)—150 μm, and d) fluorescein-loaded (10% w/w)—150 μm. Uniform solid PLG microspheres of 220 μm diameter were used to form scaffolds for sub-studies, except the scaffolds used in scanning electron microscopic imaging (160 μm set) and flow profile assessment study (dye-loaded microspheres were also used).

### 3. Particle Size Distribution

The size distribution of microsphere preparations was determined using a Coulter Multisizer 3 (Beckman Coulter Inc., Fullerton, Calif.) equipped with a 560-μm aperture. Freeze-dried particles were suspended in Isoton electrolyte that was stirred at low speeds to prevent particles from settling. A minimum of 5,000 microspheres was analyzed for each set of particles.

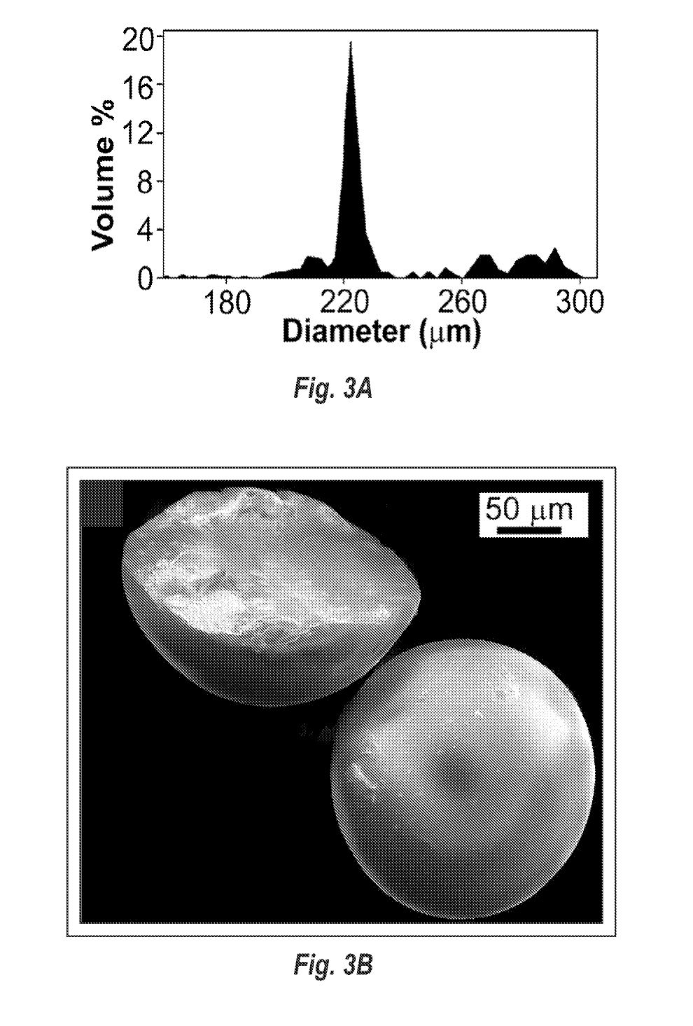

Characteristic size distribution of the microspheres revealed their relative monodispersity by the coulter multisizer size analysis distribution plot of PLGA microspheres, which shows monodispersity at a discrete peak at 220 um (FIG. 3A). In addition, scanning electron microscopic examination of the microspheres confirmed their solid interior morphology by showing the interior and exterior morphology of cryofractured microspheres (FIG. 3B).

### 4. Scaffold Fabrication

Two sets of freeze-dried microspheres were separately loaded into two syringes in the form of suspensions, prepared by suspending microspheres (˜1% w/v) in distilled water/PVA solution (volume ratio PVA:distilled water 1:20 (PVA 0.5% w/v)). The syringes were then installed in the scaffold fabrication apparatus (FIG. 1). The suspensions were pumped through the attached tubing to a cylindrical glass mold (6 mm diameter) in a controlled manner using programmable syringe pumps (PHD 22/2000, Harvard Apparatus, Inc., Holliston, Mass.). Through the bottom of the mold, the distilled water/PVA solution was filtered, while the microparticles stacked in the mold. The suspensions in the syringes were constantly stirred magnetically to keep them homogeneous. To prevent microspheres from rapid settling or sticking to the walls of the mold, a constant level of distilled water was maintained in the mold, controlled by an additional infusion syringe pump (Harvard Apparatus, Inc.) and a vacuum pump. The stacked microspheres were washed with distilled water (˜100 mL) and then were allowed to soak in ethanol (100%) for 50±20 minutes. Ethanol-soak resulted in physical fusion of adjacent microspheres, resulting in the formation of a melded matrix. The molds (containing the scaffolds) were freeze-dried (Freezone, Labconco benchtop model, Kansas City, Mo.) for a minimum of 2 days, and then the scaffolds were retrieved from the molds. In some cases, the scaffolds were prepared using suspension(s) of dye-loaded microspheres with predefined distinct flow profiles, which were later used in concentration profile assessment studies.

Melded microsphere matrices were constructed using blank microspheres of 160 μm or 220 μm diameter with 50 minutes ethanol soak-time (S₁₆₀₋₅₀ group and S₂₂₀₋₅₀, respectively). A few additional scaffolds were prepared using microspheres of 220 μm diameter and varied ethanol soak times (50±20 min). The ethanol soak-time was selected based on preliminary scaffold fabrication results, which indicated that a minimum of 30 minutes ethanol-soak was required to produce mechanically integrated scaffolds, while exceeding soaking time of 70 minutes resulted in reduced porosities. In general, the optimum range of ethanol soak-time was a function of the polymer properties (co-polymer ratio, molecular weight, etc.). Microspheres composed of PLG with lower molecular weights are expected to require shorter ethanol soak-times or dilution of ethanol for production of mechanically integrated porous matrices.

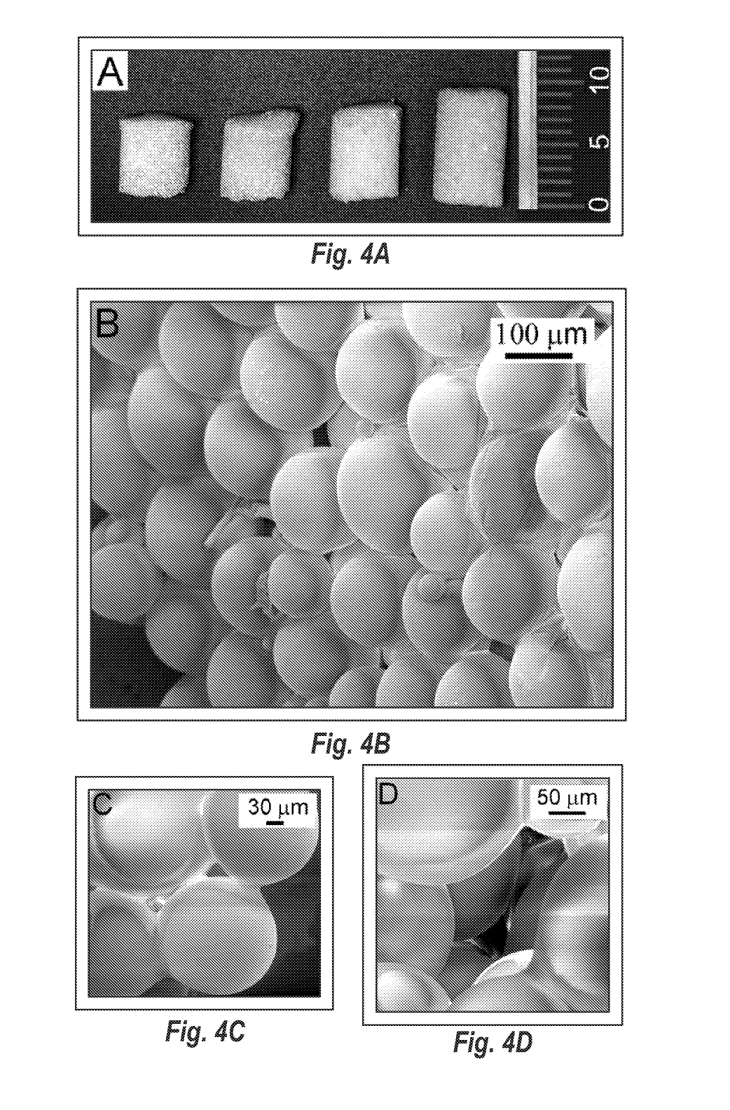

Cylindrical scaffolds, 6 mm in diameter and 4 to 9 mm in height, were prepared and their morphology was analyzed (FIGS. 4A-4D). Scanning electron micrographs of a representative scaffold from the S₁₆₀₋₅₀ group revealed that the scaffolds were porous, having interconnected pores. In addition, the effect of ethanol-soak on the microspheres was visualized, confirming that exposure to ethanol resulted in slight melting of the surface of microspheres that led to the formation of a well-connected matrix.

Changing the ethanol soak time resulted in the slight variation in the overall morphology of the scaffold. While it may be expected that an increase in ethanol soak-time would result in an increased stiffness and reduced porosity of the scaffolds, no such trends were seen in the range of ethanol-soak times examined. However, visual inspection of the scaffolds did reveal that scaffolds prepared by a 30 minutes soak did not have well-integrated microspheres, and microspheres were falling off of the ends of the scaffolds.

### 5. Scanning Electron Microscopy

Scanning electron microscopy was used to image the interior and surface morphology of the microspheres. A drop of distilled water containing suspended microspheres was placed directly onto an aluminum stub, which was freeze-dried overnight (Freezone, Labconco benchtop model, Kansas City, Mo.). The microspheres were frozen in liquid nitrogen, cryofractured with a razor blade, then sputter coated with gold before imaging using a Leo 1550 field emission scanning electron microscope at an accelerating voltage of 5 kV under a high vacuum.

To observe size, distribution and interconnectivity of the pores of the scaffolds, a dry scaffold was fractured with a razor blade and placed on an aluminum stub. The sample was sputter coated with gold and imaged using a Leo 1550 field emission scanning electron microscope at an accelerating voltage of 5 kV under a high vacuum.

FIG. 4A shows that the scaffolds can be prepared to have the shape of the mold, which was cylindrical. The scaffold was prepared with blank microspheres of 220 um diameter with a 50 minute ethanol soak time. As such, any shape of mold can be used to prepare the scaffolds of microspheres. FIGS. 4B-4D are scanning electron micrographs of a scaffold prepared with blank microspheres of 160 um diameter. FIG. 4B illustrates the outside appearance of the scaffold and shows melding of adjacent microspheres. FIG. 4C illustrates a magnified view of the melded microspheres obtained by the ethanol soaking FIG. 4D illustrates the pores that are formed in the scaffold during the melding process.

### 6. Differential Scanning Calorimetry

Differential scanning calorimetry (DSC) (Q100, TA Instruments, Inc., New Castle, Del.) was used to measure the change in glass transition temperature (T_{g}) of the PLG following the microparticle and scaffold preparations. Prior to the analysis, raw PLG and one set of microspheres were lyophilized for 1 day, and a scaffold (prepared by ethanol-soak of 50 min) was lyophilized for 2 days. The experiments were carried out in triplicate on the samples (˜15-20 mg each) packed in sealed aluminum pans. For each sample, a nonisothermal scan was performed from −10° C. to +80° C. at a heating rate of 10° C. min⁻¹ under nitrogen atmosphere, and the corresponding T_{g} was recorded.

The effect of microsphere preparation and scaffold fabrication on the glass transition temperature of PLG was analyzed by differential scanning calorimetry. The results are reported in Table 1. Microsphere preparation led to a small drop (˜1.4%) in the glass transition temperature of the raw polymer (p<0.05). However, the microsphere melding process resulted in reduced glass transition temperature of the PLG, where the average glass transition temperature dropped by 14% compared to raw PLG (p<0.05).

TABLE 1
Comparison of Glass Transition Temperatures
using Differential Scanning Calorimetry

| | Glass Transition Temperature (° C.) |
|---|---|
| Pure PLGA 50:50 | 41.62 +/− 0.37 |
| Microspheres | 41.04 +/− 0.17 |
| Scaffold | 35.68 +/− 0.30 |

### 7. Porosity Estimation

Theoretical porosities of the scaffolds were calculated using the density of the raw PLG and the apparent densities of the scaffolds prepared by 50±20 minutes ethanol-soak. The diameter (d), thickness (h) and mass (m) of the cylindrical scaffolds were measured, and porosities were calculated as:

$$Porosity = ( 1 - {ρ}_{app} / ρ ) \times 100 \% ;$$

where ρₐₚₚ is the apparent density of the scaffold, given by

$${ρ}_{app} = 4 m / π {d}^{2} h ,$$

and ρ is the density of the raw PLG (1.34 g/mL according to the manufacturer). The mean theoretical porosities of the scaffolds prepared by 30, 50 and 70 minutes ethanol-soak were found to range between 44.9 to 49%.

### 8. Mechanical Characterization

Unconfined compression tests were performed using a uniaxial testing apparatus (Instron Model 5848, Canton, Mass.) with a 10 N load cell. A custom-made stainless steel bath and compression-plate assembly was mounted in the apparatus. Cylindrical scaffold samples prepared by ethanol-soak of 50±20 minutes (5 to 9 mm in height) were tested at a strain rate of 10 mm/minute at room temperature, and moduli of elasticity were obtained from the linear regions of the stress-strain curves. The stress was defined as the ratio of the load to the initial cross-sectional area, and the strain was defined as the ratio of the change in the length to the original length. The mechanical integrity of the scaffolds, analyzed by unconfined compression testing, indicated that average moduli of elasticity of these scaffolds ranged from 4.2 to 6.0 MPa (n=4).

### 9. Porcine Ankle Chondrocytes Isolation/Culture

Porcine chondrocytes were harvested from a hog ankle (Duroc, 6 months old, female) obtained from a local slaughterhouse. Briefly, pieces of articular cartilage were retrieved aseptically, and then minced with a scalpel. Subsequently, chondrocytes were isolated from the tissue by digestion in 30 mL of 2 mg/mL sterile collagenase (type 2, 305 U/mg; Worthington Biochemical, Lakewood, N.J.) at 37° C. overnight. The cells were then plated for expansion in monolayer and incubated at 37° C. in 5% CO₂, with media changed every 2-3 days. The cell culture medium consisted of Dulbecco's Modified Eagle medium, 10% Fetal bovine serum (ES cell quantified), 1% Penicillin-streptomycin-fungizone, 1% non-essential amino acids (all from Invitrogen Life Technologies, Carlsbad, Calif.) and 50 μg/mL L-ascorbic acid (tissue culture grade; Fisher Scientific, Pittsburgh, Pa.). The cells were expanded and passaged twice before being seeded onto the scaffolds.

### 10. Cell Seeding And Viability

Cylindrical scaffolds (˜6 mm diameter) were prepared using a 50 minute ethanol soak, as mentioned earlier, and shortened with a razor blade to a height of approximately 1 mm. Cells were seeded onto these scaffolds at a density of 3×106 cells per scaffold using the orbital shaker method as described previously (Almarza A J, Athanasiou K A. Seeding techniques and scaffolding choice for tissue engineering of the temporomandibular joint disk. Tissue Eng 2004 November-December; 10(11-12):1787-1795.), and cultured for 18 days. Briefly, the scaffolds were sterilized in ethylene oxide for 12 hours and aired for one day. The scaffolds were then placed in the individual wells of a 12-well untreated plate, pre-coated with 600 μL of 2% agarose to prevent cell attachment to the wells, and incubated overnight at 37° C. in 1 mL of medium. Subsequently, the scaffolds were wetted in sterile-filtered ethanol, washed twice with PBS and once with the medium, before a highly concentrated equivalent volume of cells was dripped directly onto the middle of each scaffold. Cells were then allowed to attach to the scaffold for 2-3 hours at 37° C. After adding an additional 1 mL of medium, the well plate was allowed to stir for two days on the lowest settings in an orbital shaker at 60 rpm, and then allowed to sit for 16 days with half of the media refreshed every other day. Following this incubation period, the scaffolds were stained with LIVE/DEAD reagent (dye concentration 2 mM calcein AM, 4 mM ethidium homodimer-1; Molecular Probes, Carlsbad, Calif.) and incubated for 45 minutes, before being subjected to fluorescence microscopy (Olympus/Intelligent Innovations Spinning Disk Confocal Microscope with epifluorescence attachment).

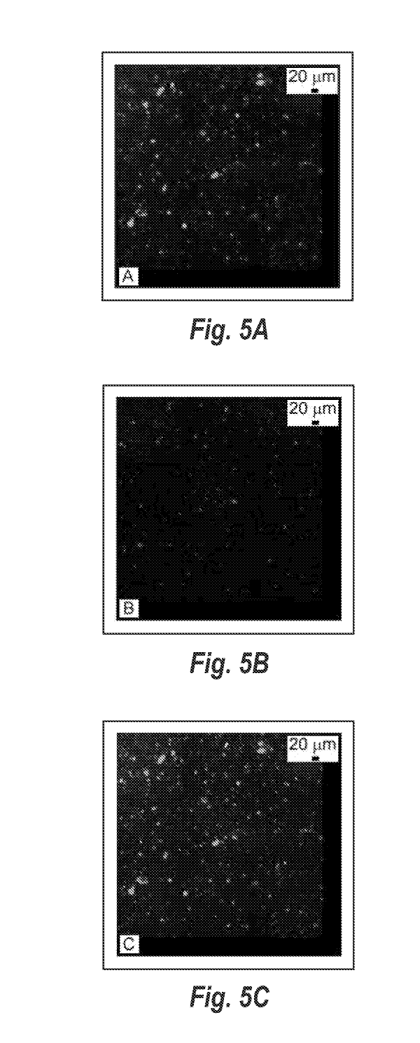

Porcine ankle chondrocytes, dynamically seeded and cultured on S₂₂₀₋₅₀ group scaffolds, were assessed for their viability. The majority of the cell population was identified as viable after a total of 18 days in culture. FIG. 5 demonstrates the results of the live/dead assay, displaying the images from a randomly selected field of view. Based on semi-automatically conducted cell counts using ImageJ software, the viability of cells was approximately 74±7% (n=3). However, as shown in FIGS. 5A-5C, some areas of dark green spots were also observed that probably were indicative of clusters of live cells. FIG. 5A shows live cells, FIG. 5B, shows dead cells, and FIG. 5C shows live and dead cells, which correlate with the cells of FIGS. 5A and 5B. The ratio of the areas stained green (FIGS. 5A and 5C, which show up as lighter spots in grayscale photos) and red (FIG. 5B-5C, which show up as lighter spots) was also calculated from the images using ImageJ software, which still led to a similar prediction of approximately 75±11% cell viability (n=3).

### 11. Concentration Profile Assessment Study

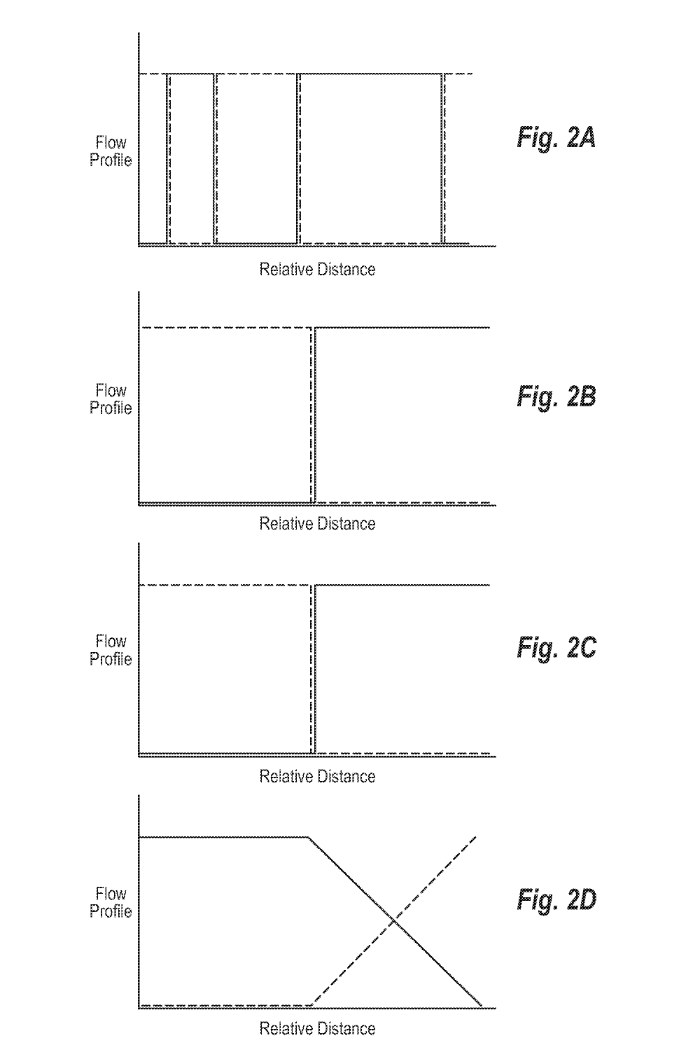

Studies were conducted to determine spatial control over composition of the scaffold. Four specific scaffolds were prepared, as mentioned earlier, using two different microsphere types (rhodamine-loaded and fluorescein-loaded microspheres, or rhodamine-loaded and blank microspheres). The syringes were loaded individually with one microsphere type and attached to the scaffold fabrication apparatus. Microspheres were pumped in a predefined manner using specific flow profiles (FIGS. 2A-2C), and then the scaffolds were prepared by melding microspheres together with an ethanol-soak of 50 minutes. Specific flow profiles were programmed into the two syringe pumps. The solid lines in the graphs of FIGS. 2A-2D illustrate the flow profile of RhodamineB-loaded microspheres with solid lines, and the dashed lines represent the flow profile of fluorescein-loaded microspheres (FIGS. 2A-2C) or blank microspheres (FIG. 2D). The resulting scaffolds were imaged under UV light using a UV lamp (254/365 nm; UVGL-25, UVP, Inc.) and a high-resolution camera (Sony Cybershot DSC-F828 8.0 MP), and images were analyzed using NIH ImageJ software to assess spatial control over the composition of the scaffolds.

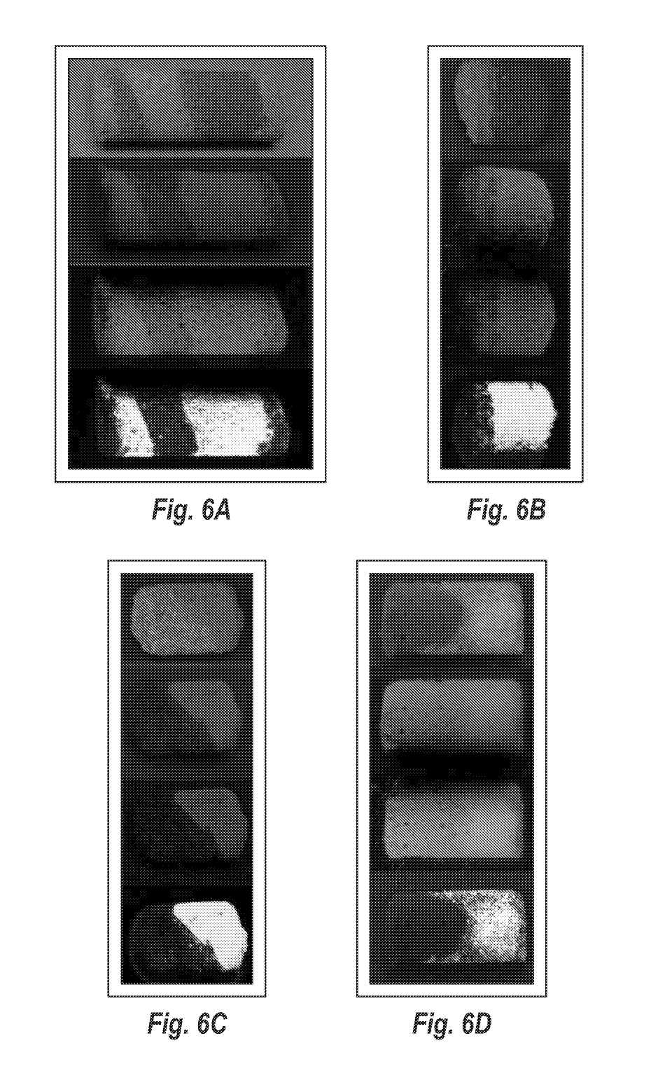

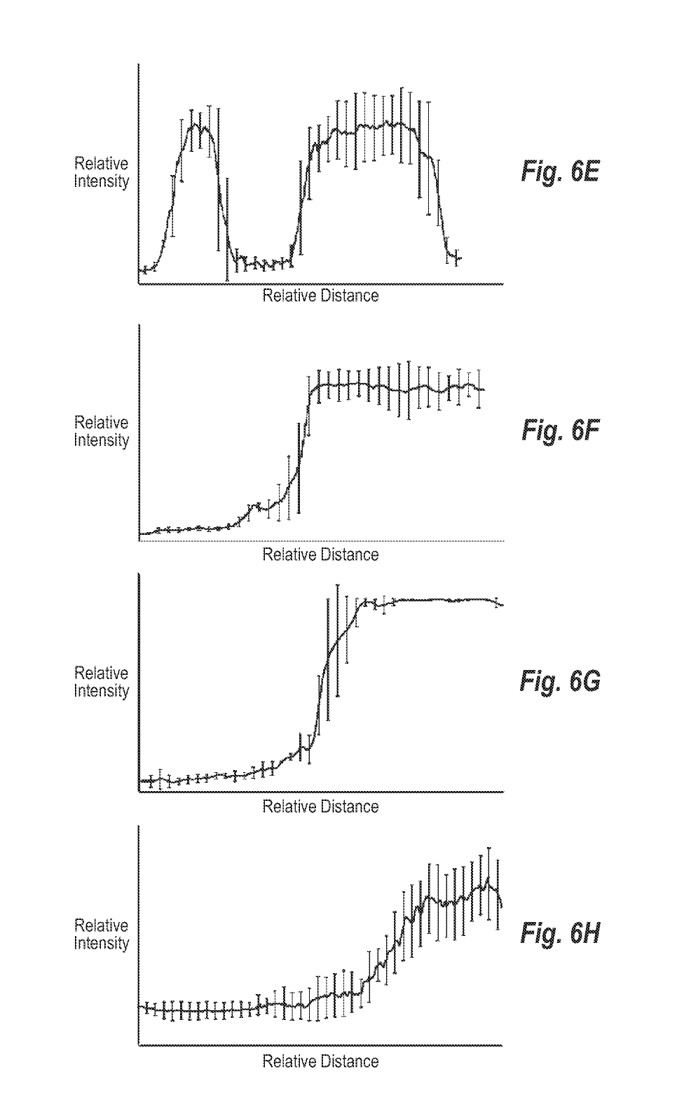

Images of scaffolds that were prepared using specific flow profiles with dye-loaded or blank microsphere suspensions (described in FIGS. 2A-2D) are shown in FIGS. 6A-6D. Images of the scaffolds captured under UV light were modified by pseudo-coloring them to create black and white images. Each image was divided in five equal parts, and particle distribution in the direction perpendicular to the interface was analyzed using ImageJ software to create relative intensity vs. relative distance plots. The plots demonstrate successful fabrication of bi-layered, multi-layered and gradient scaffolds (FIGS. 6E-6H). Irrespective of the scaffolds, standard deviations were higher at the interface, probably due to imprecise settling of the microspheres in the mold and/or wetting effects on the walls of the mold. The characteristic nature of each plot, however, was similar to the corresponding flow profile applied during the scaffold fabrication. The plots demonstrate the ability of the scaffold fabrication set-up to create scaffolds of various predefined profiles with spatial control. In addition, the orientation of the interface may also be varied (compare FIGS. 6B and 6C), which can be controlled by manipulating the vertical orientation of the cylindrical mold. Note that similar flow profiles were used to prepare these two scaffolds (FIGS. 2B and 2C), the only difference being the pitch of the mold.

FIGS. 6A-6D show the loading and concentration profiles of bi layered, multilayered, and gradient scaffolds using rhodamine B loaded microspheres and fluorescein loaded or blank microspheres. The top row of FIGS. 6A-6D show that the rhodamine B loaded microspheres (darker bands; red in color) were transitioned into fluorescein-loaded (lighter bands; orange in color, FIGS. 6A-6C) or blank microspheres (lighter; white in color, FIG. 6D). The middle rows of FIGS. 6A-6D show the scaffolds under UV light (365/254 nm), and show a characteristic change in appearance of fluorescein-loaded microspheres from the lighter color to a darker color, which was blue. The bottom row of FIGS. 6A-6D shows dark pixels (red in color) from the other images that were pseudo colored in white against a black background, and were analyzed using ImageJ software to the create relative intensity versus relative distance plots of FIGS. 6E-6H (corresponding to FIGS. 6A-6D, respectively).

### 12. Statistical Analyses

The effect of microparticle preparation and ethanol-soak on the glass transition temperature of PLG were statistically analyzed using a three-level single factor analysis of variance (ANOVA) and a Fisher's Protected Least Significant Difference post hoc test.

13. Sub-Critical Carbon Dioxide (CO₂) Melding The microsphere-based scaffold can be prepared with a sub-critical carbon dioxide (CO₂) technique. Sub-critical CO₂ is not toxic and can be used in place of organic solvents. Accordingly, poly(lactic-co-glycolic acid) microspheres of three different sizes (e.g., 100 um, 175, um, and 500 um) can be prepared. The microsphere-based scaffolds can be prepared by melding the microspheres together in a high pressure chamber at sub-critical pressures of CO₂. For example, sub-critical pressures can be 165 psi, 190 psi, and 220 psi, or any range therebetween. However, higher pressures can be used for larger microspheres, and lower pressures for smaller microspheres. Scanning electron micrographs of fractures of scaffolds displayed the porous nature of scaffolds and confirmed the successful melding of microspheres. Scaffolds were tested under compression in PBS at 37° C., and displayed an increase in stiffness with decreasing microsphere size. Human umbilical cord mesenchymal stromal cells (UCMSCs) were seeded and cultured with the scaffolds. Live/Dead cell assays, histology, immunohistochemistry, and quantitative biosynthesis assays were performed, and the results demonstrated that the sub-critical carbon dioxide (CO₂) melding technique can be used in tissue engineering, such as in bone or cartilage tissue engineering. Also, the use of moderate carbon dioxide (CO₂) melding can be used to encapsulate cells during the process of scaffold fabrication in order to preserve cell viability.

### 14. Cell Attachment And Matrix Synthesis

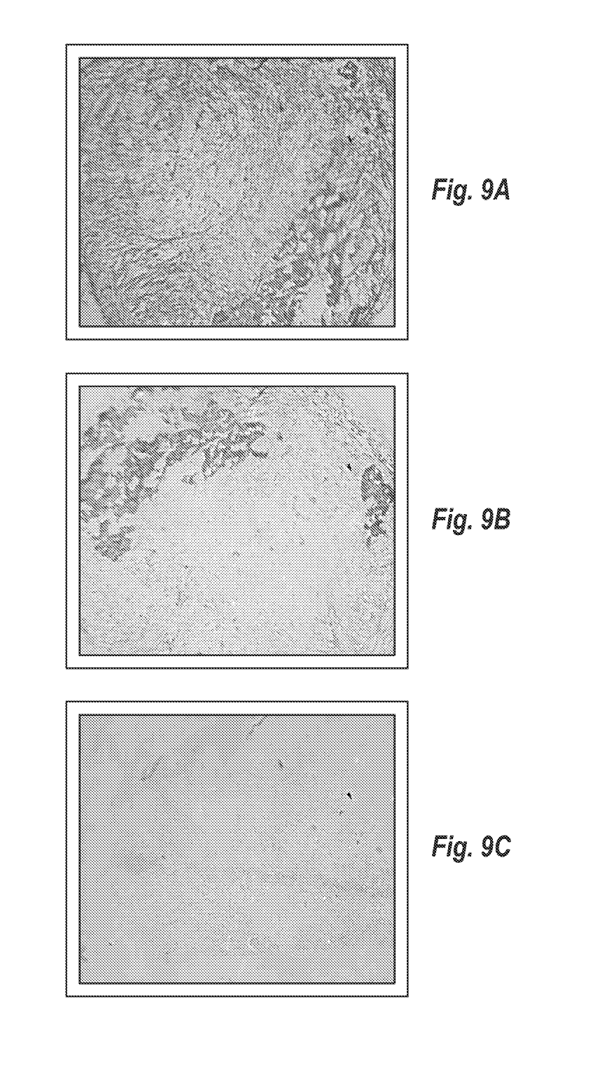

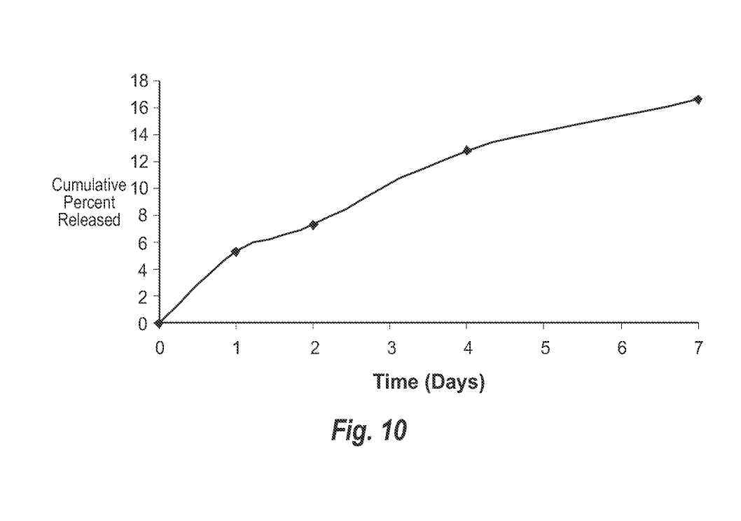

hUCMSCs were cultured on the microsphere-based scaffolds for 6 weeks global with 0.1 μM dexamethasone and 10 ng/mL TGF-β₁, and produced both collagen I and II (FIGS. 9A-9C). Additionally, rUCMSCs were seeded on the scaffolds to demonstrate that we were indeed able to harvest and seed them in the scaffolds (FIG. 10). The cells of FIGS. 9A-9C were obtained after hUCMSCs were seeded on microsphere-based scaffolds for 6 weeks in chondrogenic medium and produced the following: FIG. 9A) collagen I, FIG. 9B) collagen II; and FIG. 9C) is a negative control.

### 15. Activity of Released Protein

The tertiary structure of a common protein (lysozyme) was evaluated by fluorescence spectroscopy before and after encapsulation in PLGA microparticles by dissolving in dimethyl sulfoxide (DMSO) and obtaining a spectrum from 310-400 nm. The peak position remained the same, with the exact peak position determined to be 339 nm for both samples (data not shown), indicating that the native structure of lysozyme was retained upon extraction from the microspheres.

### 16. Growth Factor Release Profile And Activity

ELISA was used to determine the entrapment efficiency and percent cumulative release of TGF-β₁ from the particles. The entrapment efficiency of TGF-β₁ was found to be 64.3%. To determine the release, 20 mg of 150 μm particles with entrapped TGF-β₁ were placed in 1 mL of PBS (20 rpm, 37° C.). At days 1, 2, 4 and 7, the entire supernatant was collected and buffer refreshed to determine the cumulative release up to 7 days (FIG. 10). Controlled release can be obtained by altering desired parameters. FIG. 10 is a graph that shows the percent cumulative release of TGF-β₁ from PLGA up to 7 days.

### 17. Microcomputed Tomography (μCT) Imaging

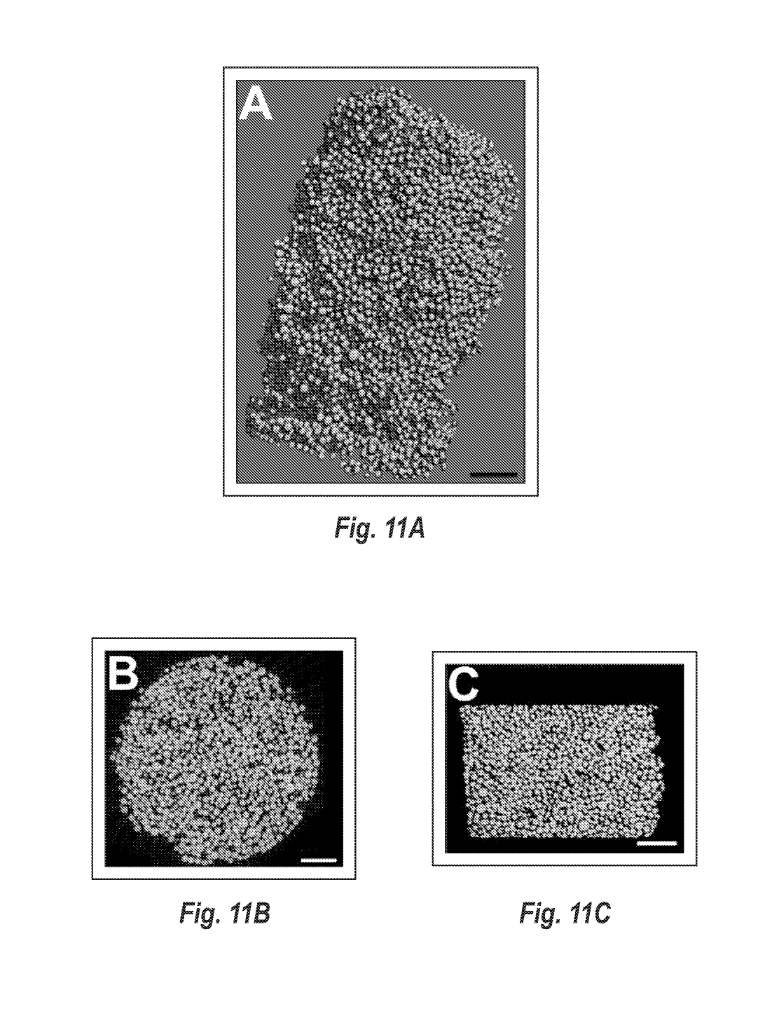

Porosity measurements for four scaffolds were performed by imaging the scaffolds with μCT (μCT 40, Scanco Medical). Using 3D reconstruction and an optimized segmentation value of 75, porosities (41.1±2.1%) and degrees of anisotropy (values were ˜1.0 for homogeneous scaffolds, i.e., isotropic) were directly determined (FIG. 11A-11C). FIG. 11A-11C are μCT images, with FIG. 11A displaying the 3-D view of a scaffold, FIG. 11B displaying an image generated from the top view, and FIG. 11C displaying an image generated from a side view. The scaffolds were analyzed using ImageJ for porosity measurement. The overall porosity (n=4) was calculated to be about 41.1% (scale bars=1 mm).

### 18. Nanophase CaCO₃ Preparation

Nanophase CaCO₃ can be prepared using a double water-in-oil emulsion technique. Briefly, two water-in-oil emulsions (A and B) can be prepared separately. In a typical procedure, the aqueous phase for emulsion A can include 0.1 M sodium carbonate, 0.2 M sodium hydroxide and 0.18 M sodium nitrate dissolved in 500 mL DI water. The aqueous phase for emulsion B can contain about 0.1 M calcium nitrate dissolved in 500 mL DI water. Oil phases for both the emulsions can be prepared by mixing 9.3 mL of Bis(2-ethylhexyl)hydrogen phosphate and 26.25 mL of sorbitan sesquioleate (Span-83) in 500 mL of kerosene. Water and oil phases for each emulsion can be mixed and sonicated at 50% amplitude for 2 minutes. Emulsions A and B can then be mixed together and stirred magnetically at 300 rpm for 30 minutes, allowing the reaction to occur. Following the reaction step, the system can be demulsified by adding 15% (w/w) ethylene glycol to the emulsion mixture. The settled CaCO3 particles can be washed with ethanol and water, successively, then sonicated at 15% amplitude for 1 minute before collection. The collected particles can then be freeze-dried for two days before being applied in the osteogenic microsphere preparation. A Beckman Coulter Multisizer III can measure the particle size and size distribution. The described method enables control over nanosphere size, with diameters ranging from ˜200-900 nm, where particle size increases with increasing calcium ion concentration (˜0.1 M to 0.25 M).

### 19. Fourier Transform Infrared (FTIR) Imaging

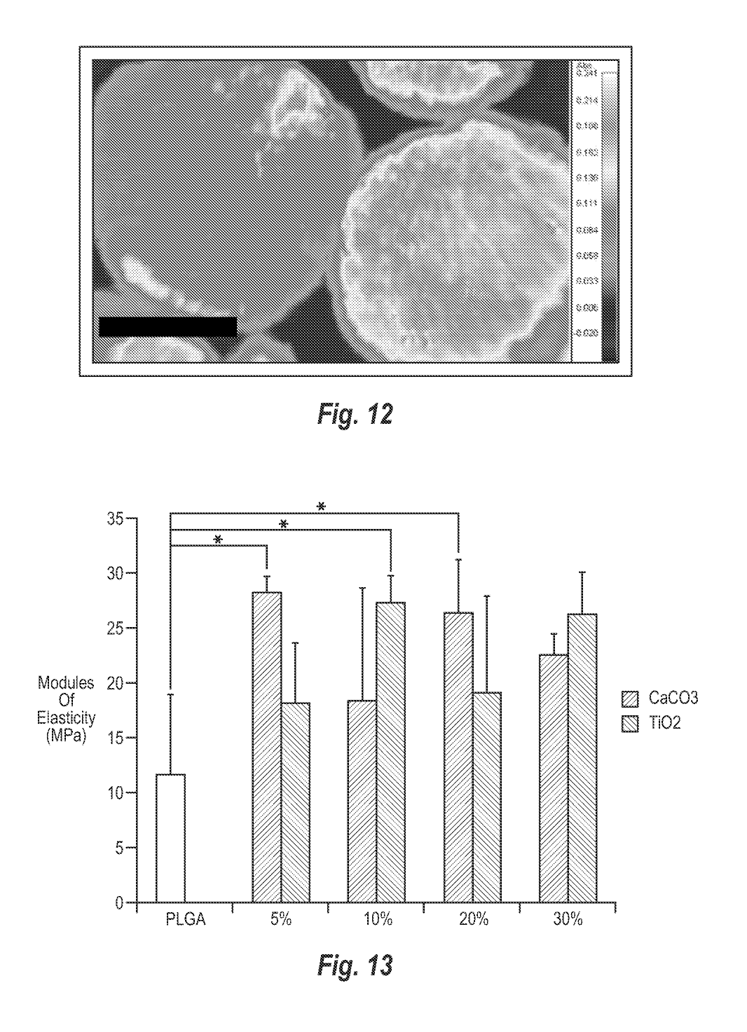

Microspheres for scaffolds, some with nanophase CaCO3 encapsulated, were imaged with FTIR (Perkin-Elmer Spotlight 400, with Spectrum 400 spectrometer; ATR mode; scan parameters: 4000˜750 cm⁻¹, spectral resolution 8 cm-1, 8 scans per pixel; spatial resolution ˜10 μm). The ATR spectral image using the intensity of 873 cm-1 band (□2-CO3) was used to identify the relative intensity of CaCO3 in the microsphere, as shown in FIG. 12. FTIR was able to clearly demonstrate the relative amount and distribution of CaCO3 in the microspheres, where microspheres with white portions have high levels of encapsulated CaCO3 In FIG. 12, the FTIR image is of 2 microspheres, with high CaCO3 (e.g., right microsphere sphere) and low CaCO3 (e.g., left microsphere) levels (scale bar=100 μm).

### 20. Mechanical Integrity of Scaffolds

Homogeneous scaffolds of either PLGA, PLGA/CaCO3, or PLGA/TiO2 microspheres were fabricated, and instantaneous moduli were determined under compression (FIG. 13). The graph in FIG. 13 illustrates the compressive moduli of PLGA, PLGA/CaCO3, and PLGA/TiO2 microsphere-based scaffolds (high strain). Composite scaffolds were markedly stiffer (e.g., the 5% CaCO3 group was 2.4 times stiffer than the pure PLGA group), which conclusively demonstrates that the nanophase materials result in a drastic increase in macroscopic mechanical integrity. This macroscopic result is comparable to AFM data, which demonstrated a 2-8 fold increase in the stiffness of PLGA microspheres with nanophase CaCO3 encapsulated.

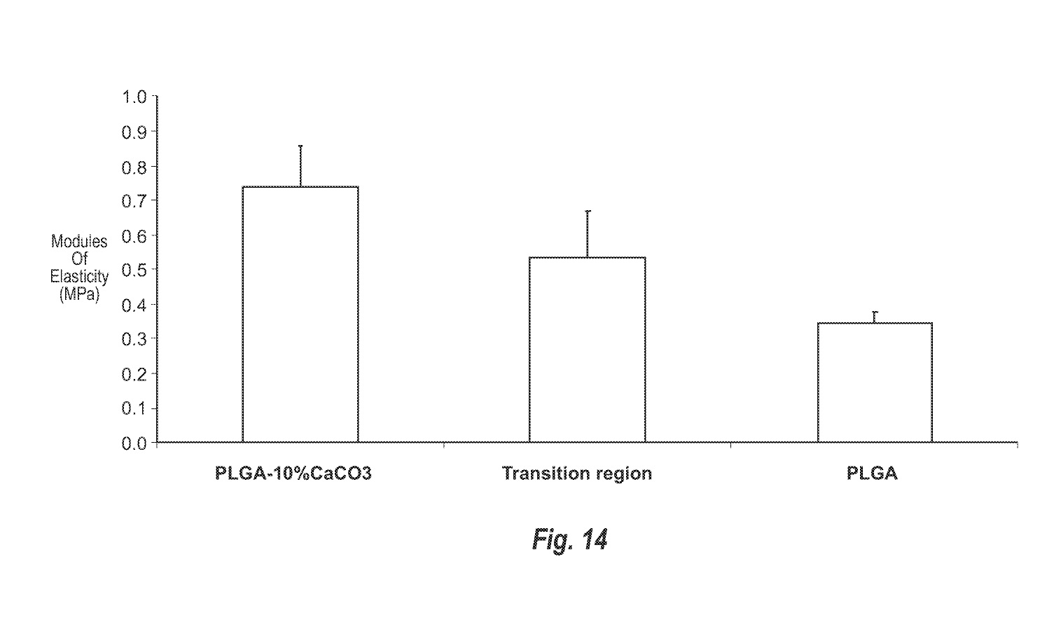

To further illustrate this stiffness gradient, exaggerated-length scaffolds (15 mm thick) were fabricated with opposing gradients of PLGA and PLGA/CaCO3 microspheres, and cut into thirds to demonstrate the drop in the average modulus of each section (FIG. 14). The graph in FIG. 14 illustrates macroscopic stiffness from three different regions of gradient scaffolds (lower strain). The composite end was 2.1 times stiffer than the pure PLGA end (p<0.001). Absolute magnitudes of stiffness vary between FIGS. 13 and 14 due to different strain levels, caused by microsphere compaction.

### 21. Microspheres Encapsulating Growth Factors

Growth factors can be tagged with a fluorescent dye in a routine manner to later monitor the maintenance of biological signal gradients of the growth factor. To conjugate dye molecules to the proteins, 10 μg of reconstituted growth factor can be mixed with 1 mL sterile PBS. Sulforhodamine B acid chloride (red color, 0.09 mg) can be mixed with dimethyl formamide (2 μL), added to the protein solution and incubated for 2 hours at 37° C. The reaction can be stopped by adding 1.5 M hydroxylamine, followed by centrifugation at 3000 g in ultracentrifugation columns for 1 hour to remove the unbound dye. A similar procedure can be followed for green fluorescent labeling with FITC dye. Uniform chondrogenic microparticles exhibiting the desired size (200 μm) can be fabricated using PLGA as a material according to techniques previously reported. An appropriate PLGA formulation (50:50 lactic acid:glycolic acid, Mw 25,000) was selected for a targeted release of 6 weeks. A typical experiment to fabricate the chondrogenic PLGA microspheres can emulsify 250 μL of a 50 mg/mL solution of reconstituted protein (TGF-β1 or TGF-β3) in deionized water into 500 mg of PLGA dissolved in 5 mL of dichloromethane. The emulsion can be formed by pulsed ultrasonication of the PLGA/protein mixture on ice at moderate power for 1 minute, a method confirmed to efficiently maintain protein structure. This emulsion can then be sprayed through a nozzle (˜250 μm in diameter) that vibrates at a frequency tuned to match the jet flow-rate and the desired PLGA/protein drop size. The emerging PLGA/protein droplets can be surrounded by an additional stream of 0.5% w/v polyvinyl alcohol solution. Extraction of the dichloromethane into the continuous aqueous phase can result in the formation of solidified PLGA particles entrapping the protein. Previous experiments have verified that negligible damage occurs to proteins as a result of shear forces occurring in the nozzle apparatus (data not shown). The particles can then be collected by centrifugation and lyophilized to remove residual dichloromethane. Similar procedures can be performed for osteogenic microparticle preparation, using either PLGA or PLGA/CaCO3 (95:5 PLGA:CaCO3 w/w), and replacing the chondrogenic growth factor with BMP-2 or other biological factor. Following the preparation of microspheres, a Beckman Coulter Multisizer III can measure the particle size and size distribution. Scanning electron micrographs (LEO 1550) of selected samples offer supportive evidence of particle size and internal morphology.

### 22. Fabrication of Stacked Microsphere-Based Scaffolds

Heterogeneous scaffolds can be constructed by assembling chondrogenic and osteogenic microspheres together. One set of freeze-dried chondrogenic and osteogenic microparticles microspheres can be separately loaded into two syringes in the form of suspensions, prepared by suspending microspheres (˜1% w/v) in DI water/PVA solution (volume ratio PVA:distilled water 1:20 (PVA 0.5% w/v)). The syringes can then be installed in the scaffold fabrication apparatus (FIG. 1). The suspensions in the syringes can be constantly stirred magnetically to keep them homogeneous. The suspensions can be pumped through the attached tubing to a cylindrical glass mold (3.5 mm diameter) in a controlled manner using programmable syringe pumps (PHD 22/2000, Harvard Apparatus, Inc., Holliston, Mass.) with the specific gradient profile consisting of a linear transitioning region. Microspheres can enter, settle and stack on the bottom of the mold (the density of particles is higher than water), while the accumulating DI water/PVA solution is filtered through the base of the mold. To prevent microspheres from rapid settling or sticking to the walls of the mold, a constant level of distilled water can be maintained in the mold, controlled by an additional infusion syringe pump (Harvard Apparatus, Inc.) and a vacuum pump. Similar procedures can be performed to create homogeneous microsphere matrices, with the only difference being that only one group of microspheres (either chondrogenic or osteogenic) will be flowed through the syringes. Also, other types of microspheres with any type of active agent can be used.

23. Differentiation of hUCMSCs

Umbilical cord matrix stem cell biology can be investigated and characterized in several applications of the microsphere-based scaffolds. Accordingly, immunology, neural application, and species differences (e.g., human, pig, dog, rat, etc.) of umbilical cord matrix stem cells can be studied with the microsphere-based scaffolds.

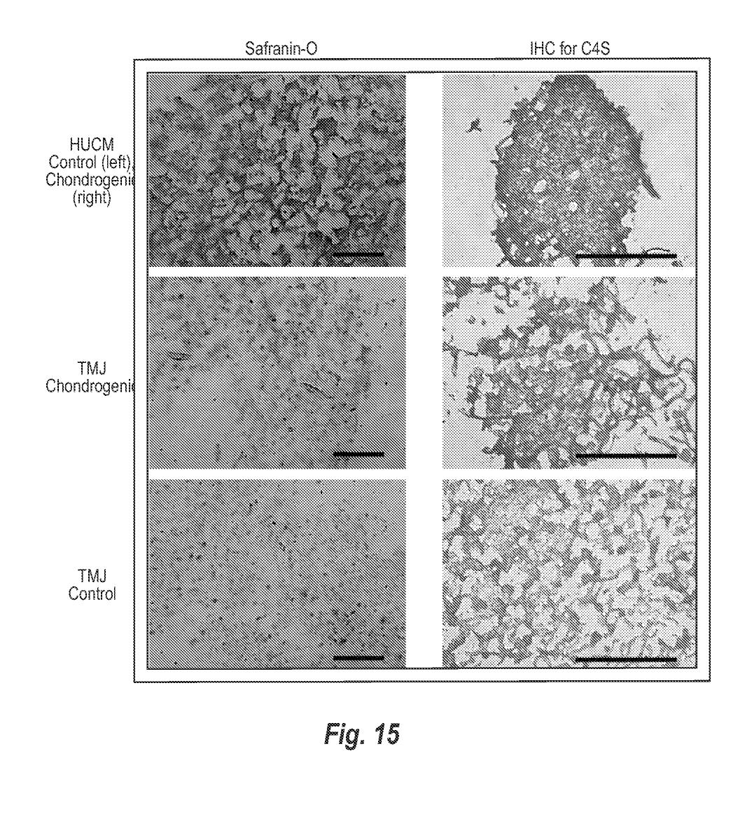

Porcine TMJ condylar cartilage cells and hUCMSCs were separately seeded onto PGA scaffolds for 6 days in spinner flasks, containing control and chondrogenic medium, respectively. After seeding, constructs were then each cultured in either control or chondrogenic medium for an additional 4 weeks. Although both groups were seeded at 5 million cells/mL, the hUCMSCs were 55% greater in number immediately after seeding and 2 times greater after 4 weeks. After 4 weeks, Saf-O/Fast green staining indicated a significant amount of GAG synthesis by hUCMSCs (FIG. 15), and immunohistochemical staining demonstrated a widespread presence of collagen I along with scattered collagen II in hUCMSC groups (data not shown). These results suggested a superiority of hUCMSCs over cartilage cells for cartilage tissue engineering. FIG. 15 show 4 weeks of chondrogenic differentiation, where the left column is safranin-O/fast green, and the right column is chondroitin-4-sulfate (C4S) immunostaining Top to bottom row are hUCMSCs in control (left) or chondrogenic (right) medium, TMJ cartilage cells in chondrogenic medium, and TMJ cartilage cells in control medium. The significantly higher intensity of staining in the hUCMSC constructs demonstrates that the hUCMSCs significantly outperform TMJ cartilage cells in GAG synthesis.

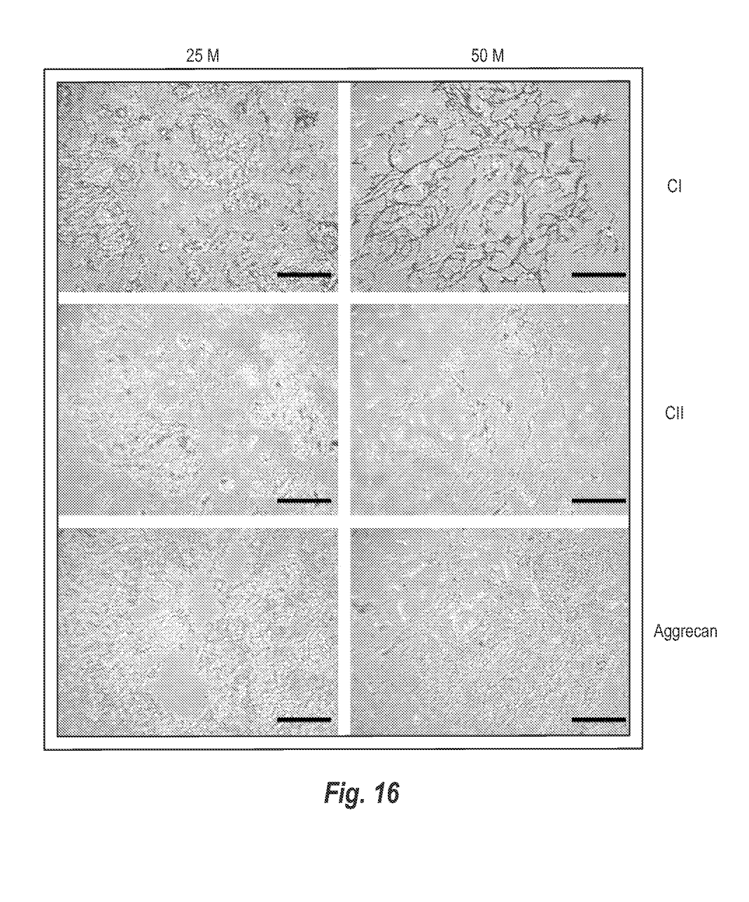

In an effort to improve collagen synthesis, higher cell densities were employed. Cells were seeded onto PGA scaffolds using an orbital shaker at 5M, 25M and 50M cells/mL. After 4 weeks, intense collagen I staining and localized areas of collagen II and aggrecan staining were observed in the high density groups (FIG. 16). Moreover, hydroxyproline assays confirmed that the 50 M group produced ˜3 times more collagen (36±2 μg collagen/construct) than the 25M group (12±6 μg collagen/construct). FIG. 16 shows IHC staining for collagen types I and II and aggrecan after 4 weeks, where 25M and 50M refer to seeding densities in cells/mL, CI and CII refer to collagen I and II, respectively.

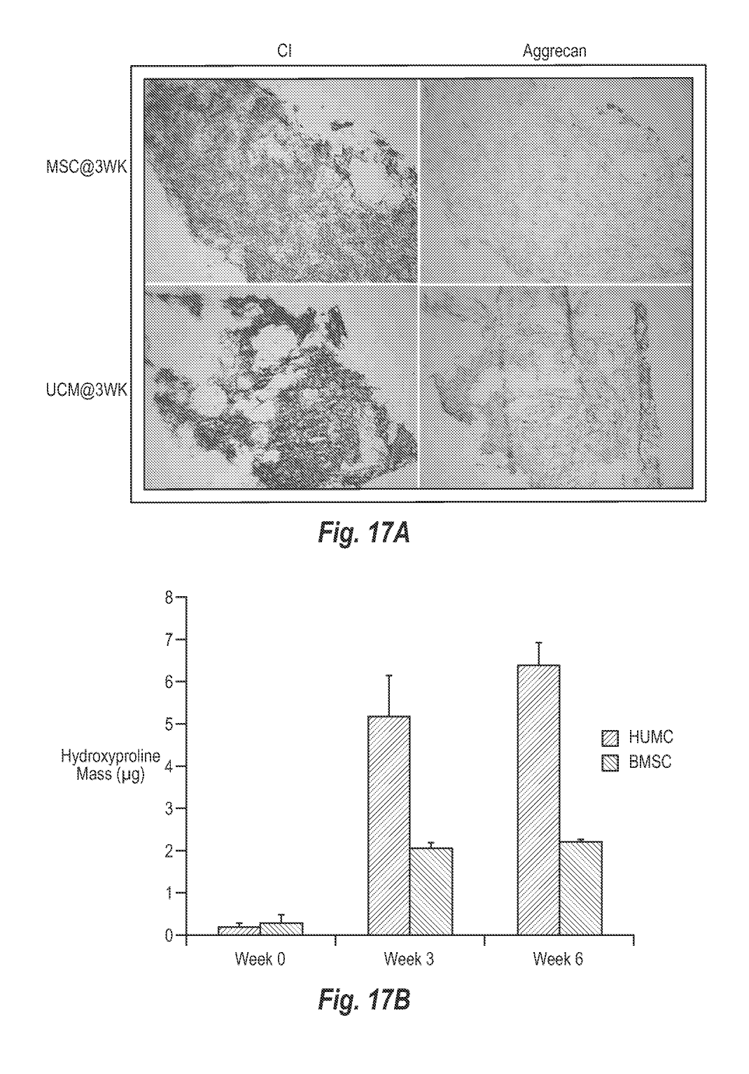

Additionally, hUCMSCs were compared to hBMSCs in vitro. P-5 cells were seeded onto PGA scaffolds using an orbital shaker, and immunohistochemical staining demonstrated a dramatic difference in the expressions of collagen I and aggrecan at week 3 between the two cell types (FIG. 17A). In addition, the hydroxyproline content for the hUCMSC-seeded group was 3 times higher by week 6 (FIG. 17B). These findings suggest that hUCMSCs may outperform hBMSCs for cartilage and osteochondral tissue engineering applications. FIG. 17A shows immunohistochemical staining for collagen type I and aggrecan after 3 weeks of culture on PGA scaffolds. FIG. 17B shows hydroxyproline content for hUCMSC and BMSCs after 3 and 6 weeks of culture, respectively.

24. Osteogenic Differentiation of hUCMSCs

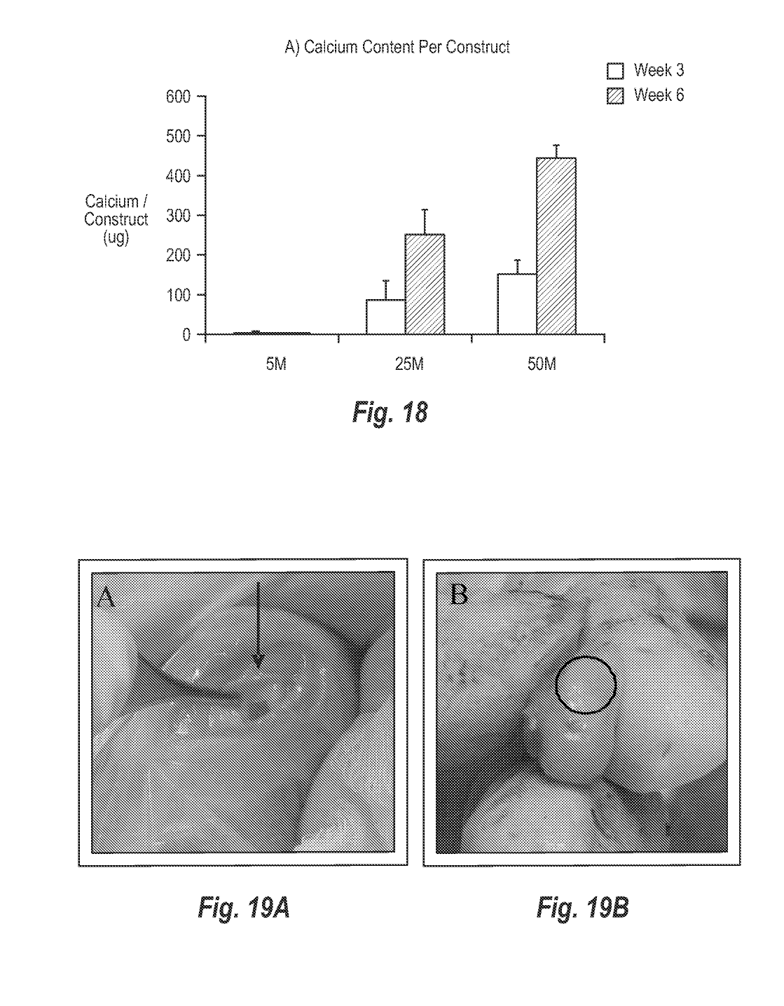

The hUCMSCs were cultured in PGA scaffolds for a period of 6 weeks in osteogenic medium. Seeding densities of 5, 25 and 50 million cells/mL were compared, and it was discovered that the highest density group produced significantly more calcium per construct (FIG. 18) and per cell. FIG. 18 is a graph that shows calcium content increased over time, and was significantly higher for higher density hUCMSC constructs. Also, more intense von Kossa staining (data not shown) was observed. These results clearly demonstrate the osteogenic capacity of hUCMSCs in 3D tissue engineering, which has never before been demonstrated in the literature. Such osteogenic capacity of hUCMSCs in 3D tissue engineering is surprising and unexpected.

### 25. In Vivo Evaluation

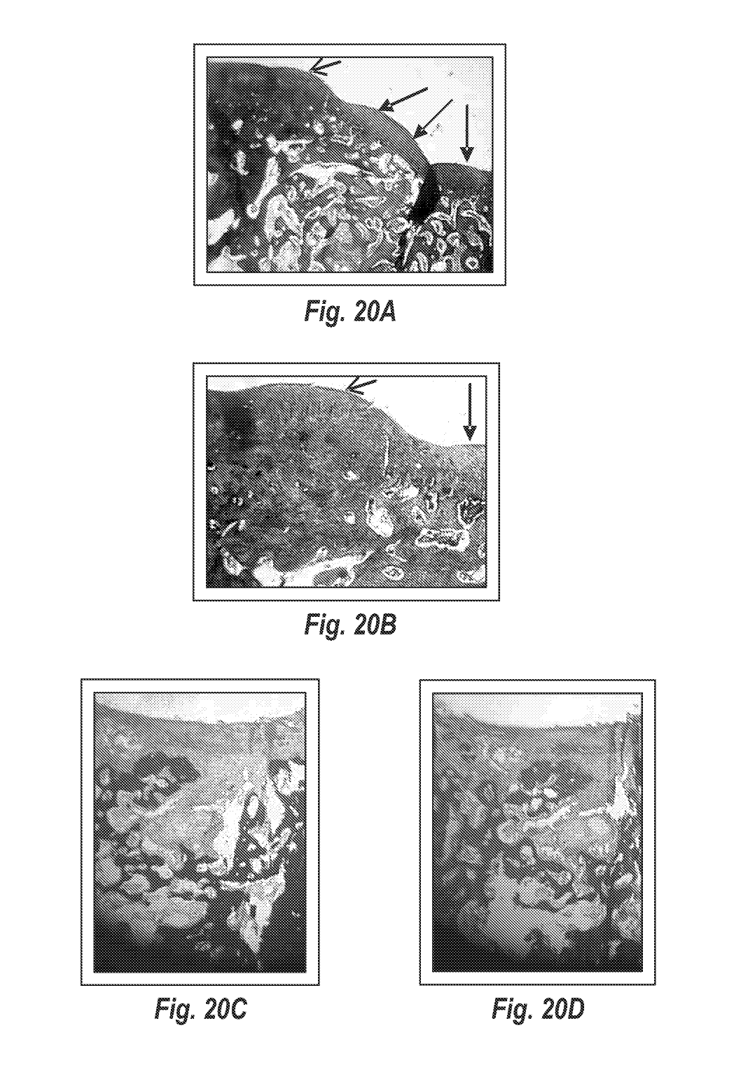

A preliminary study was performed to demonstrate the feasibility of using microsphere-based scaffolds for osteochondral defect repair. Defects (size: 3.5 mm height, 3 mm diameter) were generated in the knees of New Zealand White rabbits (male, ages 6-9 months), and PLGA microsphere-based scaffolds were surgically implanted (FIG. 19A-19B). Rabbits were sacrificed after 6 weeks, and implants were recovered for analysis. Visual inspection of the superior surface of the implant indicated a smooth cartilaginous tissue formation (FIG. 19B). The picture in FIG. 20A shows an osteochondral defect created in the rabbit knee (medial femoral defect), with the arrow showing the location of the defect. The picture in FIG. 19B shows the surface of the implant (in circle) appears to resemble a cartilaginous tissue, closely matching with the surrounding tissue.

Additionally, histological results provide evidence of regional cartilage and bone formation de novo. FIGS. 20A-D include histological data of the in vivo study after 6 weeks of implantation. FIG. 20A-20B show histological results following 6 weeks of implantation, and show Saf-O/Fast green staining of the implant/tissue. The large arrow indicates the tissue formed within the constructs. The small arrow indicates the surrounding tissue (control). FIG. 20C shows Von Kossa staining, which shows signs of mineralization in the implant. Also, FIG. 20D shows Alizarin Red staining, and also shows signs of mineralization in the implant.

### 26. In Vitro Cell Growth

Cells (e.g., human mesenchymal stem cells from bone marrow) were cultured on the a blank scaffold (e.g., control scaffold) and a gradient scaffold having opposing gradients of TGF-beta1 and BMP-2 for 6 wks. The scaffolds were constructed by melding the microspheres together with ethanol as the solvent. The mechanical integrity of the scaffolds at week 6 was very similar, reflected by their similar moduli of elasticity and relaxed moduli.

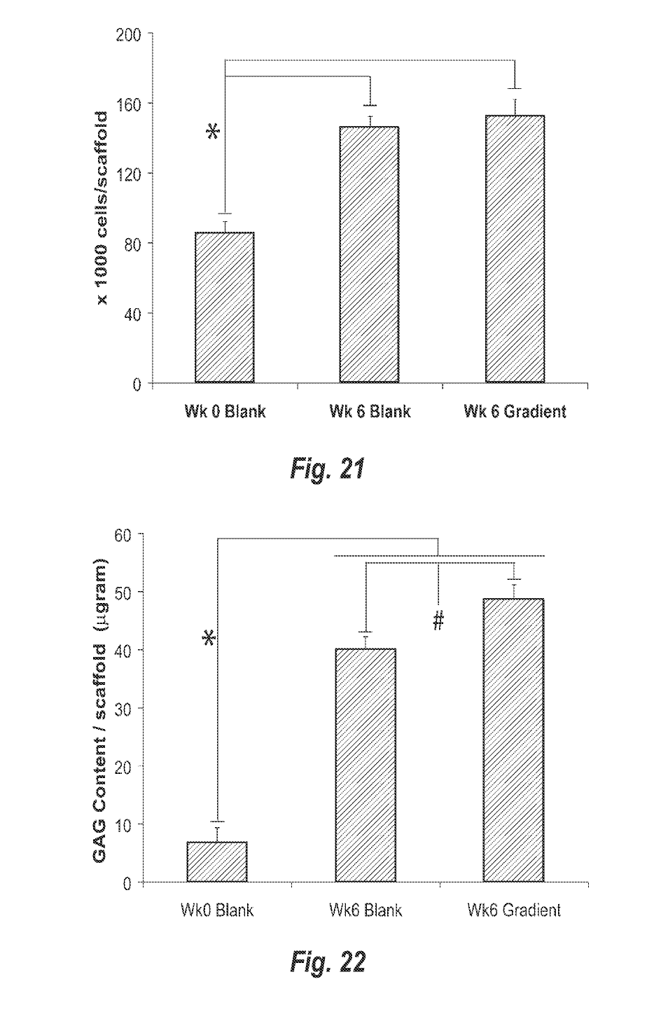

After 6 weeks, the total numbers of cells were identified per scaffold, which is shown in FIG. 21. FIG. 21 shows that both the blank scaffold and the gradient scaffold were suitable for cell growth, viability, and propagation as demonstrated by both scaffolds having a significant increase in cell numbers after 6 weeks. The overall cell number per construct increased by about 100% during the 6 week cell culture, and the differences were found to be statistically significant. Also, it should be noted that the gradient scaffold was shown to be a better scaffold.

Additionally, the amount of glycosaminoglycan (GAG) present in the cells after 6 weeks was determined. As shown in FIG. 22, both scaffolds were suitable for cell growth and GAG production. The GAG content per scaffold increased approximately 5-6 fold during the 6 week culture, and the GAG content per construct was found to be about 20% higher for the gradient scaffolds compared to the blank scaffolds Accordingly, the gradient scaffold had cells that produced significantly more GAG compared to the blank scaffold. Accordingly, the increase in GAG demonstrates that the gradient scaffold is superior over a blank scaffold. Thus, the use of microsphere gradients as described herein can be advantageous in cell culturing and tissue engineering applications.

Based on these results, the effects of chondrogenesis may be prominent during the 6 week cell culture, demonstrated by a significantly higher GAG content for the gradient scaffolds compared to the blank scaffold. An increasing trend in Alkaline phosphatase (ALP) activity for the gradient scaffold started at week 3, which may be indicative of the osteoblastic activity that began after an initial culture period. The decrease in the mechanical integrity of the constructs may be a consequence of microsphere degradation that led to the disappearance of the sintering sites with time, transitioning to the mechanical integrity of the neo-synthesized tissue.

### 27. Microsphere Size Distribution

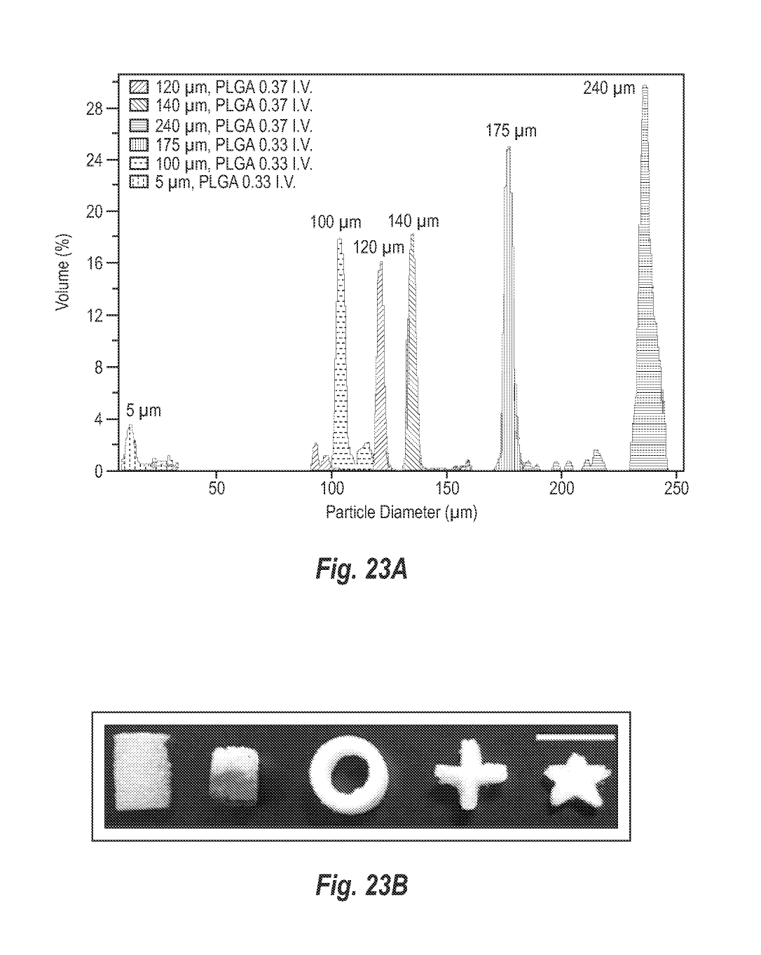

Relatively monodisperse microspheres having uniform nominal diameters were created using a previously reported method (Berkland, C., Kim, K. & Pack, D. W. Fabrication of PLG microspheres with precisely controlled and monodisperse size distributions. J Control Release 73, 59-74 (2001)). These microspheres demonstrate a solid interior morphology. The nominal particle sizes were: 120 μm, 140 μm (both with an intrinsic viscosity (i.v.) of 0.37 dL/g), and 5 μm, 100 μm, 175 μm, 240 μm and 500 μm (i.v.=0.33 dL/g) (FIG. 23A).

### 28. Scaffold Shapes

Microsphere-based cylindrical scaffolds were constructed in cylindrical plastic molds using monodisperse microspheres (˜20-80 mg) of all of the sizes except for the 5 μm group. In addition, to prepare scaffolds with a bimodal population of microspheres, a mixture of two different particle types (sizes: 5 μm and 140 μm) were used. By utilizing custom rubber molds of different shapes and microspheres of size 140 μm, a variety of shape-specific scaffolds were also constructed in a similar manner (FIG. 23B). Morphological assessment of the scaffolds using scanning electron microscopy revealed that the microsphere matrices were porous, where the microspheres largely retained their shape.

### 29. Sub-Critical CO₂ Sintering

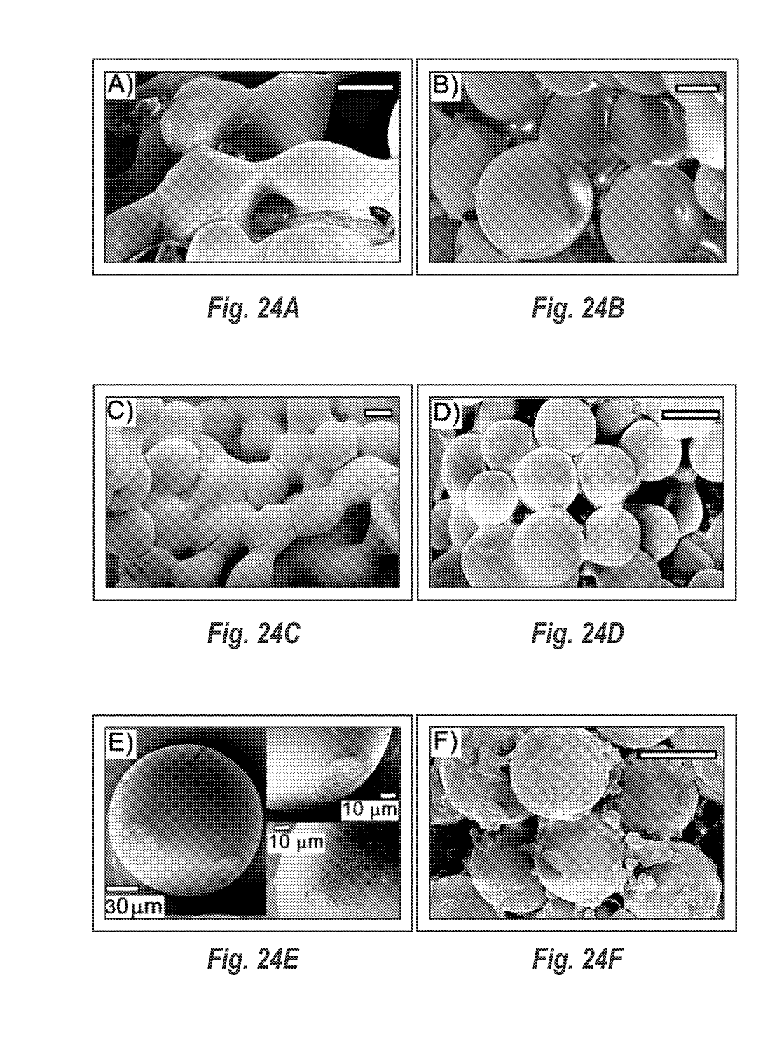

A sub-critical CO₂ sintering method was used to manufacture microsphere-based scaffolds. In the past, plasticization of PLG in pressurized CO₂ has been applied to create foamed scaffolds, where saturation of the polymer with CO₂ was performed at sub-critical pressures (˜55 bar) with equilibration periods of greater than 24 hours, and a rapid depressurization led to the nucleation of the gas (forming pores in the material) and restoration of the glass transition temperature. To prepare microsphere-based matrices in the current study, the equilibration of CO₂ in the polymer was restricted by decreasing the pressure and the duration of CO₂ exposure, leading to a comparatively reduced plasticized state of the PLG. While this allowed the microspheres to primarily retain their shape, the plasticization of the microsphere surfaces led to the sintering of the adjoining microspheres, yielding a porous matrix (FIG. 24A-24F). The conditions of CO₂ exposure are a factor for promoting the mutual-penetration and melding of microspheres, and increasing the chain mobility at the interfaces of adjoining microspheres.

The pressure (15 bar) and duration of CO₂ exposure (1 hour or less) were selected to allow sintering of all the microspheres with different sizes and i.v. of PLG. Microspheres with smaller sizes may require milder conditions, such as less pressure or shorter exposure, to achieve optimal sintering. In addition, the rate of depressurization was used to modulate the basic morphology of the scaffolds. A moderate rate of depressurization (e.g., 0.14-0.21 bar/s) was found to be optimal for the production of sintered matrices. For typical CO₂ sintering conditions, instantaneous depressurization (i.e., in less than 5 s; for 64 μm diameter microspheres, i.v.=0.33 dL/g) or depressurization at very slow rates (i.e., <0.07 bar/s; for 240 μm diameter microspheres, i.v.=0.33 dL/g) led to foaming of the prepared scaffolds, depending on the microsphere size and i.v. of the polymer.

Under the CO₂ sintering conditions, the extent of sintering of the microspheres can be a factor of the microspheres size (compare FIGS. 24A-24B with FIGS. 24C-24D, respectively). Also, the PLG microspheres of lower i.v. (i.e., 0.33 dL/g) displayed a distortion from the spherical morphology and a higher degree of sintering (compare FIGS. 24A and 24C with FIGS. 24B and 24D, respectively). Both the size of the microsphere and the intrinsic viscosity of the polymer were found to affect the pore sizes. As can be observed in FIGS. 24A-24F, the pore sizes for the scaffolds prepared with PLG microspheres of lower i.v. had anisotropic pores with closed pores at several places. Roughly, the average pore-sizes were around 70 μm (FIGS. 24A and 24B), 50 μm (FIG. 24C) and 40 μm (FIG. 24D). Micrographs of a single microsphere (140 μm) revealed the modifications in the surface of the microspheres following the CO₂ sintering, including the microsphere connection sites (FIG. 24E). The microsphere morphology, closely resembling the appearance of a microsphere in an ethanol melding method, showed the presence of a surface film of PLG containing ripples, indicating the surface plasticization of PLG. To improve the inter-microsphere connection that could improve the mechanical characteristics of the scaffolds, scaffolds were prepared using two different groups of microspheres (140 μm and 5 μm) that were mixed together in a ratio of 1:8 by weight, respectively. Additional connecting bridges between the large microspheres were formed, however, at the loss of overall scaffold porosity, with reduced pore-sizes (FIG. 24F).

### 30. Mechanical Strength

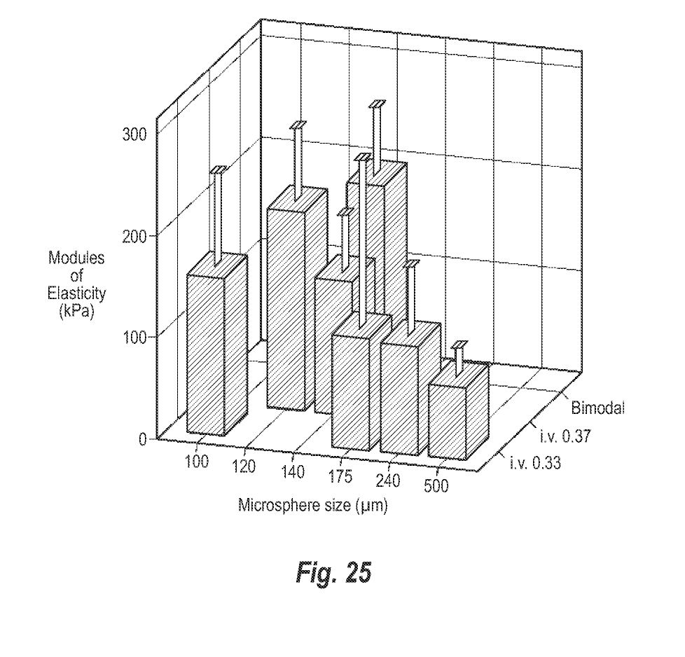

Mechanical characterization of the scaffolds was performed by unconfined compression under simulated physiological conditions. The hypothesized mechanism of compression for microsphere-based matrices is somewhat analogous to the compression of closed-foam cellular solids. Following an initial linear region, a non-linear pore collapse region follows. The moduli of elasticity were determined from the stress-strain plots using the end of the initial linear regions before the onset of non-linear region (e.g., extending to about 40% strain, in general), which indicate the scaffold elasticity. The average moduli of the scaffolds ranged from 71 to 196 kPa (FIG. 25), matching the moduli of native cartilaginous tissues. The stiffnesses of the scaffolds revealed a somewhat inverse relationship between average microsphere size and average mechanical modulus. Also, a higher intrinsic viscosity of the polymer also seemed to improve the mechanical characteristics, probably because of a spherical morphology and more ordered packing of the microspheres (see FIGS. 24A-24F). In addition, inclusion of smaller interstitial spheres in the pores can lead to an increase in the average mechanical modulus (e.g., compare i.v.=0.37 vs. bimodal for the 140 μm diameter microspheres) of the scaffolds.

### 31. Cell Cultures

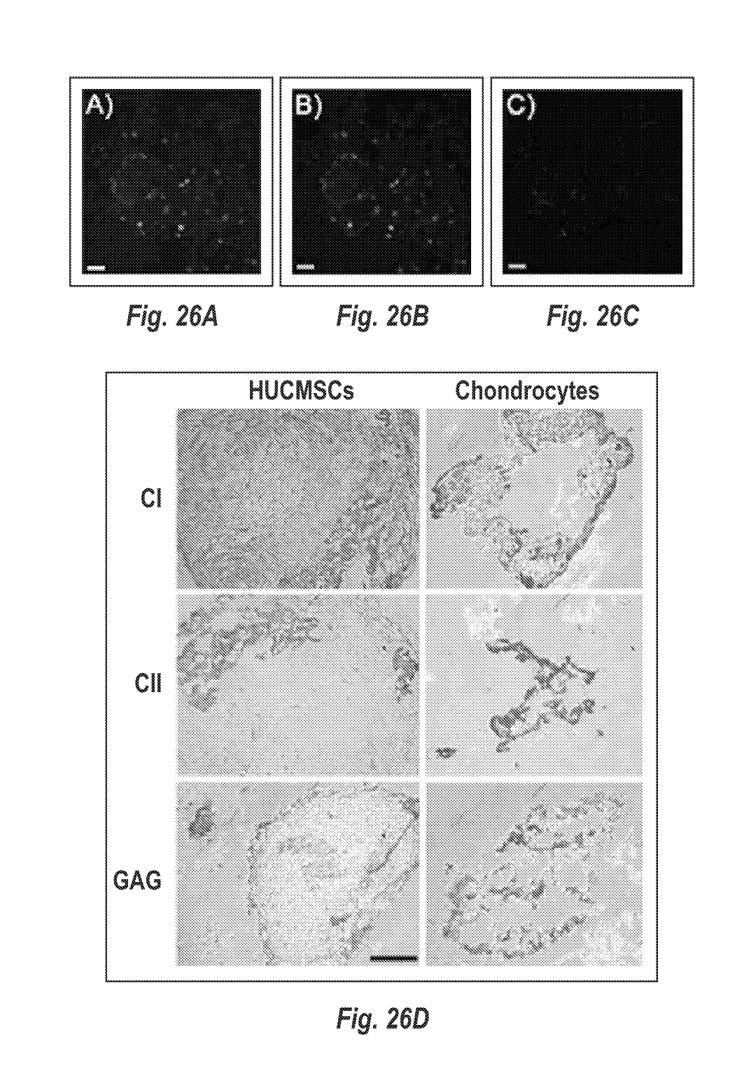

Cell culture studies were performed to determine the suitability of these scaffolds for tissue engineering. Porcine chondrocytes, dynamically seeded and cultured on the scaffolds, were assessed for their viability. The majority of the cell population was identified as viable after 3 weeks in culture (FIGS. 26A-26C). Immunohistochemistry revealed positive staining for collagen types I and II following the 3 week culture (FIG. 26II). In addition, Safranin-O staining revealed signs of glycosaminoglycan (GAG) formation for both the groups (FIG. 26D). Biochemical analysis also revealed positive indications of cartilage-like matrix formation, where the presence of GAGs and collagen were detected (Table A).

TABLE A
Biochemical Assay Results Following 3 Week Culture

| Scaffold Group | # Cells | GAG content (μg) | Hydroxyproline (μg) |
|---|---|---|---|
| Chondrocytes | 5.8 × 10⁴ | 12.8 | 1.8 |
| HUCMCSs | 5.9 × 10⁴ | 2.8 | 1.8 |

### 32. Simultaneous Sintering and Cell-Loading

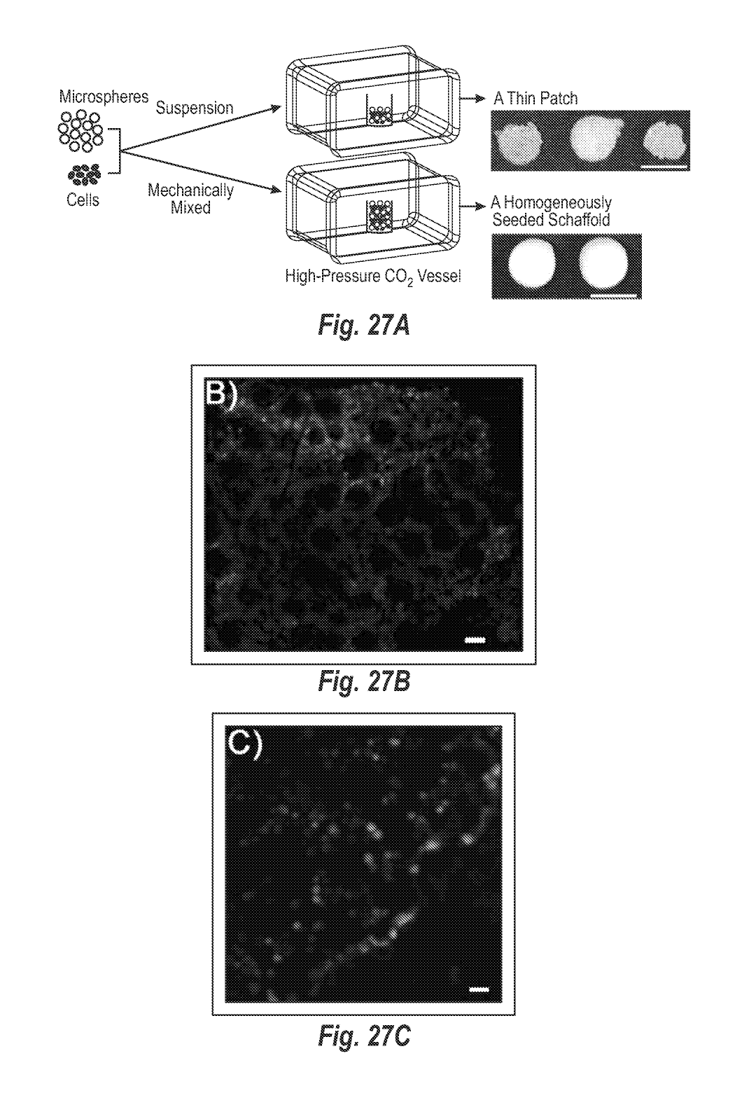

To allow for homogeneous seeding of the constructs, cell loaded matrices were fabricated via a one-step CO₂ sintering of microspheres with the HUCMSCs. The conditions of sintering were altered to minimize the time of exposure (e.g., 4 min or less, excluding the depressurization time), while keeping the CO₂ pressure to a relatively low value (e.g., 30 bar). Interestingly, when performed in the presence of the culture medium, the sintering process resulted in a thin patch formation, where only a few microsphere layers at the top of the mold were sintered together (FIG. 27). In contrast, in the absence of the medium, a mixture of cells with the microspheres yielded completely sintered matrices. The difference between the thin patch formation (e.g., with culture medium) and full 3D scaffold formation (e.g., absence of medium) can be attributed to the thermodynamic limitation of CO₂ solubility in the liquid phase (Henry's Law).

Viability assessment of the cell-loaded thin patch and the scaffolds revealed that virtually the entire cell population survived the sintering process (FIG. 27B-27C). Although CO₂ at high pressures for long durations may not be cytocompatible due to known sterilization efficacy of supercritical CO₂ achieved by lowering the cytoplasmic pH from the formation of carbonic acid and the shear forces of intercellular bubble formation upon depressurization, it was demonstrated that the milder conditions with milder gaseous CO₂ conditions are highly conducive to cell viability. Based on the size of the microspheres, the type of PLG, and the type of cells under consideration, various sub-critical CO₂ sintering conditions may exist (i.e., a number of combinations of sub-critical pressures and exposure times), which allow for the formation of cell-loaded matrices without affecting the cell viability.

### 33. Microspheres With Nanophase Doping

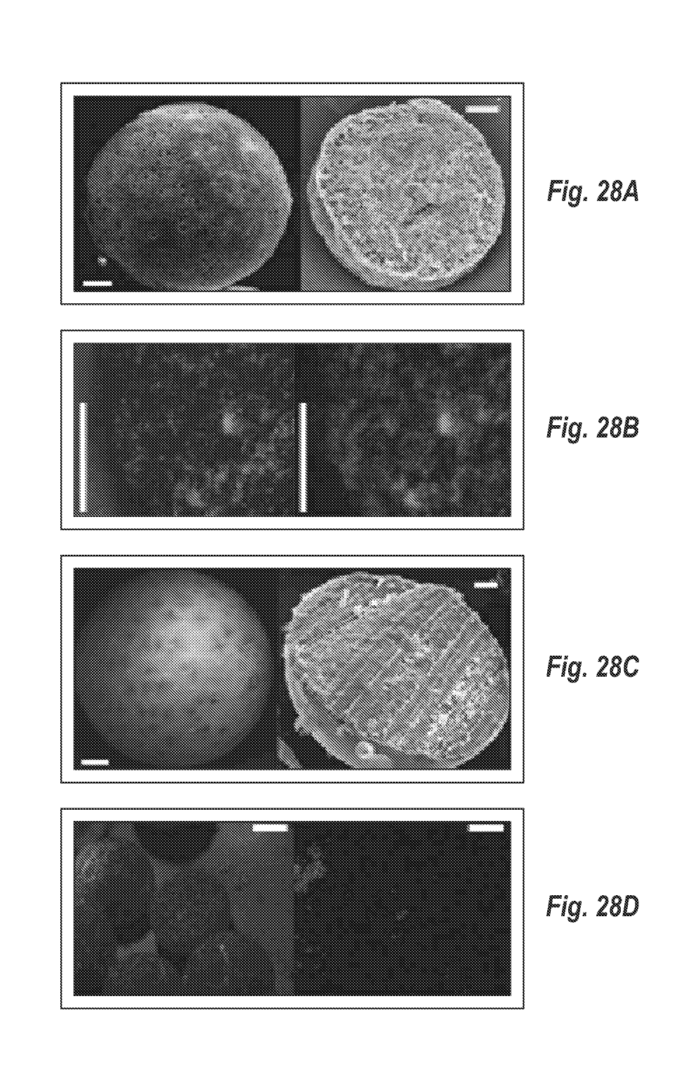

The morphological characteristics of microspheres doped with various nanophase materials were studied. Morphological analysis of microspheres was conducted using scanning electron microscopy/energy dispersive spectroscopy (SEM/EDS). FIGS. 28A and 28C display representative SEM images of intact (left) and cryofractured (right) microspheres, corresponding to 90:10 PLGA:CaCO3 and 90:10 PLGA:TiO₂, respectively. FIGS. 28B and 28D show the elemental distribution of the microspheres obtained using EDS, displaying an overlay of C, O, and Ca/Ti (left) and corresponding Ca/Ti distribution (right).

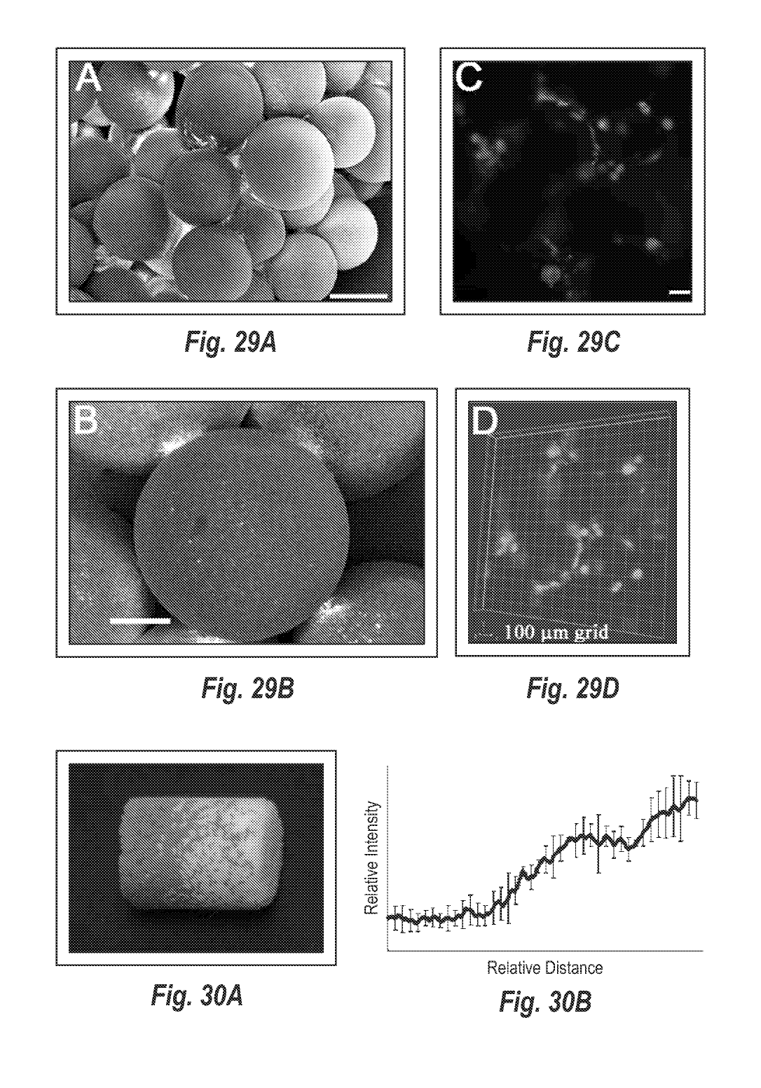

FIGS. 29A-29C include characteristic SEM micrographs of a scaffold, prepared by sintering the microspheres (90:10 PLGA:CaCO₃) using ethanol sintering. FIG. 29A shows the porous nature of the scaffold, and FIG. 29B shows microsphere connection sites. FIGS. 29C-29D include live/dead images of human umbilical cord mesenchymal stromal cells cultured on these scaffolds for a period of 2 weeks, demonstrating high viability. The representative images of cells in a single plain (FIG. 29C) and a 100 μm thick section (FIG. 29D) were taken from an interior section of a scaffold. This shows a significant number of live cells, which is beneficial for tissue engineering applications.

FIG. 30A is an image of a gradient scaffold prepared using dye (Rhodamine B)-loaded PLGA microspheres and 90:10 PLGA:CaCO₃ microspheres using a 2 hour ethanol soak. The image was taken under UV light using a UV lamp (254/365 nm; UVGL-25, UVP, Inc.) and a high-resolution camera, and analyzed using NIH ImageJ software to plot relative intensity as a function of pixel distance. The image shows that the CaCO3 doped microspheres can be prepared in concentration gradients as described herein. FIG. 30B confirms the gradient by showing the relative distance of the microspheres from one end, by measuring the relative intensity.

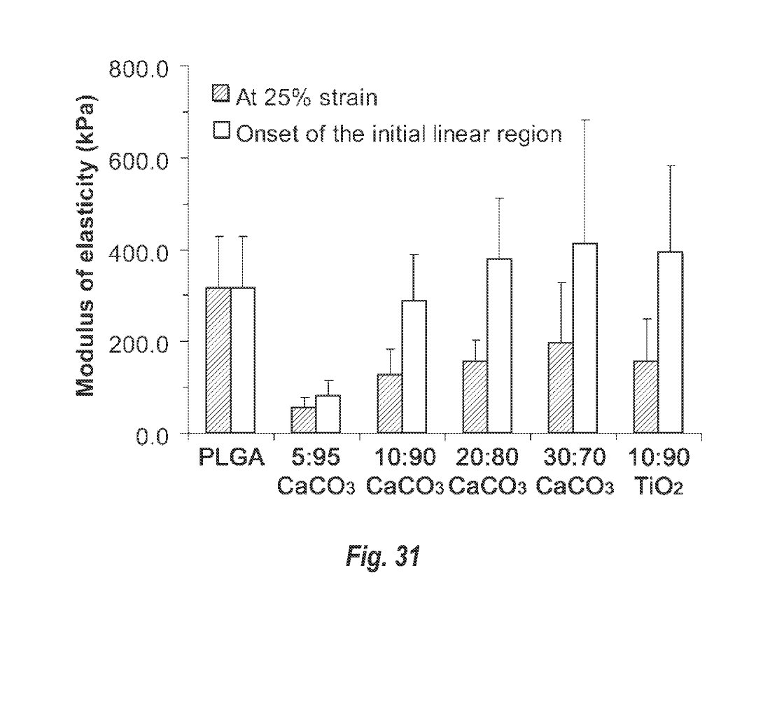

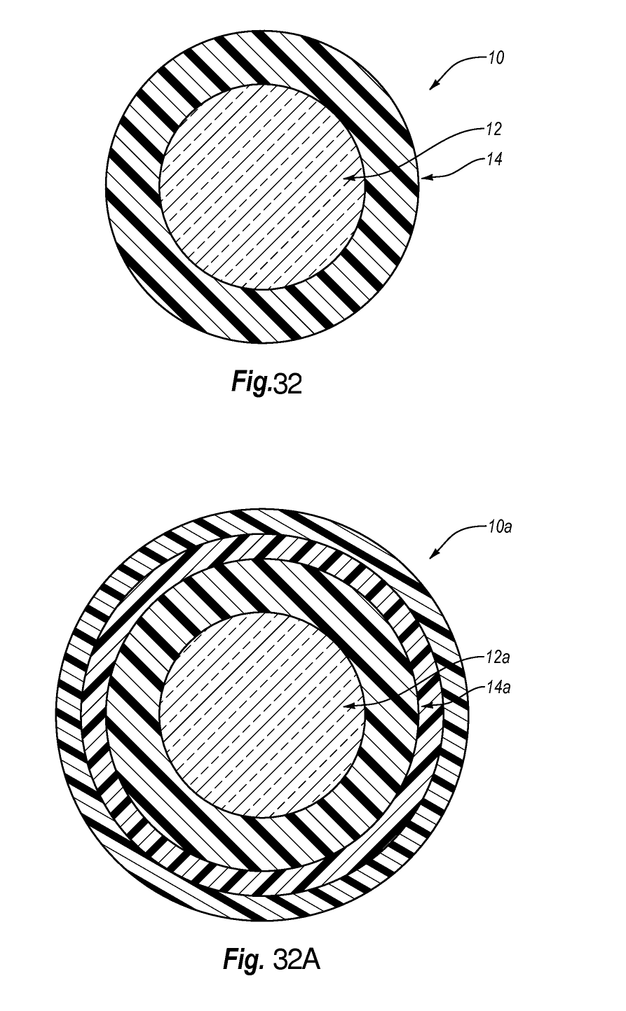

FIG. 31 is a graph that shows the moduli of elasticity of the homogeneous scaffolds prepared using different types of microspheres. The moduli were obtained from the initial linear regions of the stress-train curve: 1) at 25% strain (preceding the onset of pore-collapse for PLGA scaffolds), and 2) preceding the onset of pore-collapse, in general (at 40% strain for composite scaffolds). Surface modifications that result due to the incorporation of nano-phase materials led to a decrease in the extent of sintering of the composite microspheres compared to the control PLGA microspheres for a 2 hour ethanol-soak.

Core and Shell

The present invention is related to core and shell engineered particles (e.g., nanoparticles or microparticles) and implant tissue engineering scaffolds prepared from the particles as well as methods of making and using the same. That is, any of the scaffolds, such as those with microspheres, can include microspheres that have a core and shell configuration. The core can be harder than the shells. The engineered particles can include a hard or rigid core with one or more polymeric coatings on the core. The polymeric coating can include various agents, such as therapeutic agents, biological agents, or the like. The implants can be prepared into any shape with the particles bound together selectively at discrete locations or as melded masses. The hard core can provide stiffness and rigidity to the implant, and the polymer coating can allow for enhanced bonding and implant formation without using high heat, and as such the implants can include bioactive agents that would otherwise be susceptible to degradation at elevated temperatures. Melding of the polymeric shell can associate adjacent particles so as to form implants in any suitable shape and porosity. Also, the particles can range in size from nano-sized to micron-sized particles. The particles can range in shapes from irregular shapes to uniform shapes, but often will be in the form of a sphere or generally spherical so as to be generally referenced as nanospheres or microspheres. While the present invention is generally described in the context of microspheres, the particles may not be spherical and may be microparticles or the like. Also, while the term “microspheres” refers to size in the scale of microns, the engineered particles may have sizes in the nanoparticle range and may be nanospheres or the like.

The implants formed from the core and shell particles can be considered to be bioengineering scaffolds as the implants can be sufficiently porous to allow for cellular migration and penetration therein in order for the implant to function as a cell culture scaffold in vivo. The porosity of the implant can be modulated as described herein. The hardness of the core of each particle makes these implants especially suitable for bone scaffolds for bone regeneration, and may even be useful for orthodontic scaffolds for tooth regeneration. While the uses of the core and shell particles are generally described in connection to tissue engineering, it is understood that the engineered particles of the present invention can be applied to other fields and can be included in other articles of manufacture.

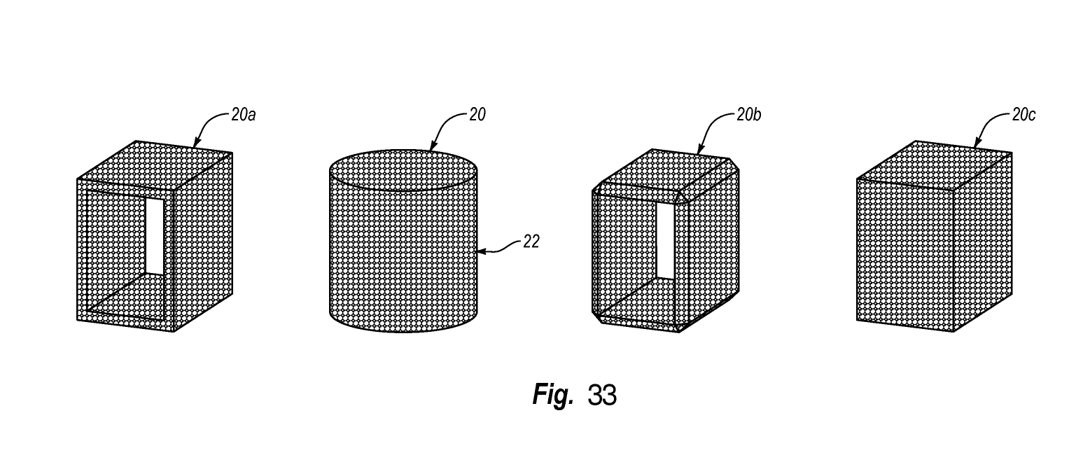

FIG. 32 shows an embodiment of an engineered particle 10 having a core 12 and a single polymeric shell 14. FIG. 32A shows a particle 10a having a core 12a and a plurality of polymeric shells 14a, each shell 14a being of the same polymeric material or of different polymeric materials. FIG. 33 shows different embodiments of an implant 20 (e.g., frame 20a, beveled frame 20b, and cube 20c) formed from the particles 22 of FIG. 32-32A.

While not shown, the implants 20, 20a, 20b, and 20c can be formed from one type of particle or a mixture of two or more different types of particles that have different parameters for a characteristic. This may include the different types of particles having different spatial distributions, such as in gradient distributions. For example, one type of particle with a first characteristic can be distributed in a body so that a first end has a majority of the particles with the first characteristic and an opposite second end has a majority of a different second type of particle. The first type and second type of particles can have gradient distributions with respect to each other, and with respect to the first and second ends of the body.

However, the particles can be prepared into any basic or complex shape with various types of surface and internal features. The shapes can have overhangs, undercuts, apertures, holes, pores, porous networks, conduits, channels, or other feature. The different shapes can be molded and/or selectively formed with any of a variety of manufacturing processes that can selectively sinter adjacent particles together by melding the polymeric shells of adjacent particles together.

The particle-based scaffold can be prepared into substantially any shape by preparing a mold to have the desired shape or by using point specific laser melding. For example, the particle-based scaffold can be prepared into the shapes of rods, plates, spheres, wrappings, patches, plugs, depots, sheets, cubes, blocks, bones, bone portions, cartilage, cartilage portions, implants, orthopedic implants, orthopedic screws, orthopedic rods, orthopedic plates, and the like. Also, the particle-based scaffolds can be prepared into shapes to help facilitate the transitions between tissues, such as between bone to tendon, bone to cartilage, tendon to muscle, dentin to enamel, skin layers, disparate layers, and the like. The particle-based scaffolds can also be shaped as bandages, plugs, or the like for wound healing.

In one embodiment, the mean particle size of the particles used to prepare the scaffolds can have a range between about less than 1 μm to about greater than 1 mm, more preferably about 100 μm to about 300 μm, and most preferably from about 180 to about 240 μm. An example of particle size is about 180 μm to about 220 μm.

In one embodiment, the core size of the particles can vary in order to vary stiffness or hardness of the implants. Size of the core particle can be in a range of about 5 μm to about 1200 μm, more preferably from about 50 μm to about 500 μm, and most preferably from about 100 μm to about 200 μm. In some instances, the core of the particle can be larger than about 1200 μm.

In one embodiment, the shell thickness can be varied to a thinner size in order to facilitate particle-particle melding such that the thin shell can result in an increase in stiffness or hardness as there is less polymer. The range of the thinner shell coating can be from about 0.025 μm to 125 μm, more preferably from about 0.05 μm to about 75 μm, and most preferably from about 0.25 μm to about 50 μm. Also, a range of the ratio of the coating thickness/core diameter can be between about 0.005 and 0.25, more preferably between about 0.01 to about 0.15, and most preferably between about 0.05 and about 0.1.

In one embodiment, the shell thickness can be varied to a thicker size in order to facilitate particle-particle melding while retaining bioactive agent in the shell, such that the thick shell can result in a decrease in stiffness or hardness as well as increase in bioactive agent loading potential as there is more polymer. The range of the thicker shell coating can be from about 1.5 μm to about 750 μm, more preferably from about 3 μm to about 600 μm, and most preferably from about 4.5 μm to about 550 μm. Also, the range of the ratio of the coating thickness/core diameter can be between about 0.3 and 1.5, more preferably between about 0.6 to about 1.2, and most preferably between about 0.9 and about 1.1.

In one embodiment, the invention can include a new material, based on high-stiffness particles (e.g., nanospheres or microspheres) having a hard core (e.g., hydroxyapatite, bioactive glass) that are coated with a polymeric material (e.g., poly(lactic-co-glycolic acid)). These hard core and polymeric shell engineered particles can be sintered together to form articles that include a singly type of particles as well as in articles with other types of particles in order to create a macroscopic implant that can be used as bioengineering scaffolding biomaterial. Accordingly, the hard core and polymeric shell engineered particles in an implant may include: the same polymer material and high-stiffness core material; different polymers; different high-stiffness core materials; and/or may be a polymeric particles without the high-stiffness material inside as a solid particle or multi-layered or multi-shelled particles. The polymeric shells of each engineered particle can be formed of a single type of polymer or different types of polymers, and each individual shell can be a single type of polymer (e.g., homo polymer or co-polymer) or mixture of different polymers. The scaffolding biomaterials may be made into different shapes based on the mold that the particles are placed into with the polymeric shells melded together, which may include one or more different types of engineered particles. Particles with different types of properties, such as different hard cores and/or different polymeric shells) can be selectively arranged in a mold in order to prepare an article with different properties at different locations. Also, point-specific laser sintering can be performed in order to prepare complex shapes by selectively melding the shells of certain adjacent particles together. Interfaces between polymeric shells that can meld together can be used at locations of an article that are solid, while polymeric shells that do not meld together (e.g., under a certain melding process) can be used in locations of an article where the particles are later removed or retained as free-flowing or movable particle powders or retained as a particles in a matrix of another matrix material. Particles that are on an internal portion of the article may not be melded together so that they can be removed from the article prior to implantation.

The present invention provides the surprising and unexpected result that it is indeed possible to use a polymeric material to coat hard particles of hydroxyapatite or bioactive glass. Moreover, it is now possible to form an implant from particles that have hard cores with properties provided by hydroxyapatite and/or bioactive glass. For example, the properties of hydroxyapatite and/or bioactive glass can be useful for implantation into bone for bone repair or bone regeneration. Particles of hydroxyapatite and bioactive glass are commercially available, and can be made into “composite” materials of particulate hydroxyapatite with polymers, with or without surface modification of hydroxyapatite nanoparticles. However, hydroxyapatite requires extremely high temperatures for sintering into a three dimensional structure, and thereby is not suitable for containing many bioactive agents or biological materials. The implants of the present invention provide for surprising and unexpected results of having a stiff implant scaffold that includes bioactive agents, such as proteomic growth factors, that can be released.

Now, however, individual high-stiffness engineering particles (e.g., hydroxyapatite, bioactive glass core with polymer shell) can each receive their own individual polymer coating with or without a bioactive material and can be sintered or melded into complex shapes for use as bioengineering implants with suitable porosity. Examples of the core material can include bioactive glass and hydroxyapatite; however, other hard materials that are biocompatible can be used, such as biocompatible metals or other ceramics or hard plastics.

The high-stiffness microparticles can be configured as particles that are individually coated with a polymeric coating so that the individual engineered particles can be used as particle, such as in a particle powder. The polymeric shell is not a matrix polymer that encapsulates multiple cores. Each engineered particle can have a single core with its own unique shell.

The high-stiffness particles themselves are stiffer that conventional polymeric particles used for implants, and correspondingly the macroscopic implant scaffolding biomaterial prepared is also stiffer that traditional polymeric particle-based implants. However, the particles and resulting implant have properties of polymeric materials in that they can be melded at low or room temperature with solvents and may be prepared to include bioactive materials, such as nucleic acids or proteins, which are not denatured or degraded by the scaffold manufacturing process. Moreover, the polymeric shell coating allows for controlled release of the bioactive materials from the scaffold once implanted.

It has now been found that a particle made of a polymer-coated hydroxyapatite particle (i.e., a single sphere of hydroxyapatite, with a polymeric shell of one or more polymeric coatings) is significantly stiffer than either a particle of equivalent size and shape made of either the polymer alone or a composite of the polymer and particulate hydroxyapatite in a bulk polymer matrix. As used herein, the shell has substantially the same of the core or more spherical and rounded with only one particle therein, where a matrix is a bulk polymer with a plurality of particles therein. This means that an implant scaffolding biomaterial made of the hard core and polymeric shell engineered particles is stiffer as well. Therefore, an implant scaffolding biomaterial intended for bone regeneration can be made stiffer than a material made of either the pure polymer particles or the composite particles, and can be made into a specific shape (e.g., to replace a segmental defect).

In one embodiment, the microparticles can be sintered in a manner that results in interstitial spaces between the polymeric shells so as to create or maintain packing pores. As such, the implant is not completely solid from side to side because there are spaces formed between the shells. The shells do not melt to form a solid matrix. The sintering can be used to control porosity and the degree of melding of the adjacent particles. That is, the particles are sintered together so that the polymeric shells of adjacent particles partially meld together to leave interstitial spaces between the melded particles and to leave general porosity. One example of sintering can be with solvent liquid or solvent vapor sintering, where the liquid or vapor of a solvent is flowed through the implant in order to meld the polymeric shells of adjacent particles to each other and to leave interstitial spaces therebetween. Some examples of the solvents that can be used in liquid or vapor sintering can include ethanol, ethanol-acetone, dense-phase carbon dioxide, chloroform, methylene chloride, or the like. The vapor melding can sinter the microparticles at a faster rate than composite particles made of the polymer with nanoparticulate hydroxyapatite. Such a sintering process is usable for core and shell microparticles as well as a mixture of core and shell microparticles with regular polymeric microparticles.

In one embodiment, the polymer used to form the shells of the microparticles can include an active agent, such as a drug, growth factor, or the like. The shells can be configured for controlled release of the active agent. As such, one or more shell layers can include the active agent, and one or more outer shell layers can be over the active agent-containing layers to control diffusion and ultimate release from the microparticles. As opposed to a scaffold made of only hydroxyapatite or bioactive glass, the polymeric component makes controlled release of bioactive molecules straightforward. This enables a high-stiffness implant scaffolding biomaterial with controlled release of active agents.

In one embodiment, two or more types of engineered particles can be used and prepared to have a corresponding uniform distribution and or relative gradient between the different types of particles. That is, the two or more different types of particles can be arranged in a gradient format with respect to each other, and then sintered into an implant having the particle gradients. This allows the sintering of particles in a gradient based design where the high-stiffness material is not desired on one side of the scaffolding biomaterial. This can be useful for scaffolding biomaterial intended for osteochondral (bone+cartilage) tissue regeneration, where the present invention allows for polymeric coated hard particles and polymer-only particles to be sintered together in a gradient design.

While the present invention has been described with respect to hard implants that are useful for bone regeneration, the invention may also be used for regeneration of any hard tissue, such as tooth regeneration. The therapeutic method can use biostable polymeric materials or biodegradable polymeric materials. When biodegradable, the resulting bone, tooth, or other hard tissue that is regenerated can retain the hard cores therein. The hard cores in the regenerated tissue can provide structural support.

In one embodiment, the implant scaffold includes a biomaterial that is made of only one type of particle that has a hard core material and a polymeric shell material.

In one embodiment, the implant scaffold has a substantially homogenous distribution of a first type of particle that has a first core material and a second core material. Optionally, the implant can have substantially homogenous distributions of any type of particle, whether a core and shell particle or a uniform polymeric particle. In one aspect, the implant can be devoid of having a gradient distribution of the first type of particle. That is, the implant is substantially uniform with respect to the first type of particle and possibly substantially uniform with respect to any type of particle included therein.

In one embodiment, the implant prepared from the hard core and polymeric shell particle has a stiffness in the mega-Pascal (MPa) range. In another embodiment, the implant prepared from the hard core and polymeric shell microparticle has a stiffness in the giga-Pascal (GPa) range. In one example, the implant can have a stiffness of about 1 MPa to about 200 MPa when the implant is dry or substantially without water or other solvent of the polymeric shell material, or from 2.5 MPa to about 150 MPa, or from 5 MPa to about 125 MPa, or from 7.5 MPa to about 100 MPa. In one example, the implant can have thin polymeric shells and have a stiffness of about 1 GPa to about 20 GPa when the implant is dry or substantially without water or other solvent of the polymeric shell material, or from 3 GPa to about 15 GPa, or from 5 GPa to about 12 GPa, or from 7.5 GPa to about 10 GPa. In one embodiment, the average moduli of elasticity of the scaffolds can have a range between about 0.5 MPa to about 75 MPa, more preferably about 1 MPa to about 50 MPa, and most preferably from about 5 MPa to about 10 MPa.

In one embodiment, the implant is prepared using laser sintering. The laser beam is focused so as to heat at least a portion of the polymeric shell thereof in order to cause melding of the adjacent particles in the path of the laser in order to link adjacent particles together. The use of laser sintering can allow for different layers to be sintered with point specific melding. Laser sintering allows for specific points or specific regions within a bulk of particles in a mold or elsewhere, such that a specific and unique shape can be formed, such as having overhanging features and under-hanging features. In one example, laser sintering can be used to prepare a shape that has gaps or holes extending through otherwise solid portions. During the laser sintering protocol, the portions that are not desired to be sintered and that are retained as flowable particles (e.g., not sintered or melded together) can be removed by allowing the un-melded or free particles to flow out. A specific example includes preparing an implant with features similar to as shown in the implant that appears like a window pane. Thus, laser sintering with point specific melding can be used to create complex shapes with complex holes or other features that are not capable of being molded.

In one embodiment, the laser sintering can be computer-controlled. That is, a CAD program, used on a computer, can be used to design an implant, and the computer can control the laser to sinter the microparticles into the shape provided by the CAD program. In one aspect, a CT image of a subject can be used to create a template for an implant to fit within a defect of a subject, such as a bond defect, and the laser sintering process can then custom sinter the microparticles into an implant to fit within the subjects-specific defect.

In one embodiment, the laser sintering protocol can be used for controlling macroporosity or general holes, conduits or apertures as well as recesses that extend into or through the sintered body. Microporosity is the small pores or interstitial space between adjacent particles and macroporosity is a hole, conduit, or aperture that extends into and through the body of the sintered body. One area can be laser sintered with small macropores while another can be larger macropores. For example, this selective macroporosity can be useful for preparing a mandibular condyle, which has one portion with regular porosity from interstitial space between particles and the other from laser-created macropores. The scaffolds can have a macropore, hole, conduit or aperture sizes ranging from about 40 μm to about 1650 μm. For cartilage tissue engineering, the macropore hole, conduit or aperture sizes can be from about 70 μm to about 120 μm. In bone tissue engineering, macropore hole, conduit or aperture sizes as small as about 50 μm or larger can be used. Optimal macropore hole, conduit or aperture sizes may be within a range of about 100 μm to about 600 μm.

In one embodiment, the implant can be prepared using one of solvent sintering, liquid solvent sintering, vapor solvent sintering, CO₂ sintering, or laser sintering. In one aspect, general heat sintering, such as performed in an oven with a heater, is generally avoided or not used to prepare the implants due to general heating deactivating or degrading bioactive agents. However, general heat in an oven can be used when the particles are devoid of a bioactive agent that denatures or degrades under heat. Heat from a laser, however, is suitable in many instances. When no heat can be used, solvent liquid or solvent vapor or CO₂ sintering can be performed.

In one embodiment, the hard core, such as bioactive glass or hydroxyapatite, can be spray coated with a polymeric solution to prepare one or more shells to the particle. The polymeric solution can include a bioactive agent, and the spraying can be done at low or room temperature in order to retain activity of the bioactive agent. The spray coating can be via traditional spray coating of particles, and may include inject coating.

In one embodiment, the coating protocol is accomplished by a circulating fluidized bed (CFB), and can provide for discrete coated particles. This is distinct from methods where numerous particles are encapsulated within a given polymer, e.g., the encapsulation of numerous hydroxyapatite nanoparticles inside of a given polymer particle. In the present invention, each polymer coating encapsulates exactly one stiff particle, and a result is a powder of polymer coated particles. The CFB technique can produce discrete coated particles, such as coated microspheres, where each particle only has one hard core.

In one embodiment, the sintering of microparticles into an implant can be accomplished by any of the following methods: heat sintering (increase the temperature), which is less desirable when bioactive agents or other temperature sensitive components are included; CO₂ sintering (high pressure CO₂, with or without temperature increase, which depresses melting point of the polymer and thus allows the polymer to melt together at a lower temperature than accomplished with heat sintering); solvent sintering (immersion in a liquid solvent such as acetone, ethanol, methylene chloride, or a combination of the above), solvent vapor sintering (same as the previous one, but vapors instead of immersion); and SLS, which leads to 2nd level porosity.

In one embodiment, the present invention provides a process to make a particle-based scaffold that is stiffer than polymeric particle-based scaffolds and that includes bioactive agents such as growth factors. The core of the particles can include hydroxyapatite, tricalcium phosphate, calcium carbonate, titanium dioxide, or the like, which core is encapsulated inside of the polymer shells. A stiffer implant can be obtained by a hard core (e.g., sphere) coated with an outer layer that is polymeric and capable of melding and formation of three dimensional implants. Accordingly, the individual sphere units that are stiffer result in the bulk implant material also being stiffer.

Mechanical testing data show that we have an increase in bulk material stiffness of about two orders of magnitude relative to bulk materials with the polymer particles alone. For example, with the biomaterial scaffold based on sintered PCL-coated particles of glass, compressive moduli in hydrated conditions at 37 degrees Celsius were approximately 3.7 MPa or about 10 MPa, compared to scaffolds made exclusively of PCL particles, with moduli under these same testing conditions of about 50 kPa. In comparison, some PLGA microsphere scaffold can have values between 142-308 kPa. The present invention can provide scaffolds with about two orders of magnitude higher stiffness for the coating-based scaffolds.

The particles when packed together in a mold present a common stacking observed with sphere-shaped objects, and with particles of similar diameter this may correspond to a void space or porosity (1st level of porosity or microporosity) of approximately 40%, depending on the extent to which the particles are sintered (in an extreme case, the microporosity would be 0% if sintered excessively to the point of being melted together completely into a solid with no pores), but typically approximately 40%. This value is the 1st level of porosity (microporosity), which can range from about 10% to about 90%, from about 20% to about 80%, from about 30% to about 60% and from about 40% to about 50%. In one embodiment, the mean theoretical porosities of the scaffolds can have a range between about 10% to about 95%, more preferably about 40% to about 60%, and most preferably from about 45% to about 50%. An example of porosity is about 44.9% to about 49%.

The selective layer sintering (SLS), such as through laser sintering for point specific sintering, allows for the particles to be sintered together without the need for complete packing, instead allowing “struts” of sintered particles to be connected to each other, and the distance between these struts on a larger length scale presents a 2nd level of porosity or macroporosity. This can be used to form solids that have recesses, holes, macropores, or apertures therein or extending therethrough.

In one embodiment, the present invention provides a method of coating a stiff particle with a softer polymeric material, and the individual particles are then linked together by virtue of the polymer coating melding with the polymer coatings of the other coated particles.

In one embodiment, the stiffer core may be a bioceramic such as a bioactive glass (any one of a wide variety of possible formulations) or hydroxyapatite, or any calcium-based bioceramic, or the like. In one aspect, the polymer coating may be a polymer such as polycaprolactone, PGA, PLA, or PLGA, or any other polymer capable of being merged (sintered/melted/dissolved). The shell allows for growth factors to be encapsulated therein. The particles can release a growth factor encapsulated in the polymer. Example growth factors might be insulin-like growth factor-I (IGF-I), any of the transforming growth factors (e.g., TGF-beta1, TGF-beta3), and any of the bone morphogenetic proteins (e.g., BMP-2, BMP-7). The particles may also contain and thereby release natural extracellular matrix materials such as chondroitin sulfate, hyaluronic acid, collagen, or the like.

In one embodiment, different types of particles (e.g., different polymer coating, core material, released growth factor or material, etc.) can be sintered together, either in a controlled fashion as in particle gradients that are described in the incorporated patent applications or simply mixed together, e.g., homogeneously.

In one embodiment, the hard core and polymeric shell coated particles can be used in a free-flowing composition. That is, the particles can be affirmatively not sintered together. As such, the particles can be used as a powder, or be introduced into a liquid solution or paste. Also, the particles can be included in liquid suspensions, gels, pastes, and other formats. The particles may also be included in solids, such as when in a solid polymer matrix with or without pores.

A tissue engineering scaffold prepared with the hard core and shell particles can be configured for growing cells, and can include a plurality of biocompatible particles linked together to form a three-dimensional matrix with interstitial spaces or pores between adjacent particles. The matrix can include a plurality of pores for growing cells. The biocompatible particles can include only a single type of particle, or can include first and second sets of particles. The first set of particles can have a first characteristic (e.g., hard core and polymeric shell), and a first predetermined spatial distribution with respect to the three-dimensional matrix. The second set of particles can have a second characteristic (e.g., only polymeric without a hard core) that is different from the first characteristic, and a second predetermined spatial distribution that is different from the first predetermined spatial distribution with respect to the three-dimensional matrix. Additional characteristics can include composition, polymer, particle size, particle size distribution, type of bioactive agent, type of bioactive agent combination, bioactive agent concentration, amount of bioactive agent, rate of bioactive agent release, mechanical strength, flexibility, rigidity, color, radiotranslucency, radiopaqueness, or the like. The scaffold includes at least one hard core and polymeric shell engineered particle as described herein.

In one embodiment, the scaffold can include a first bioactive agent contained in or disposed on the particles (e.g., hard core and polymeric shell particles). The scaffold can be configured to release the first bioactive agent so as to create a first desired spatial and temporal concentration gradient of the first bioactive agent. Optionally, a second set of different particles (e.g., different hard core and polymer shell or only polymeric) can be substantially devoid of the first bioactive agent, or can include a second bioactive agent. When the second bioactive agent is contained in or disposed on the second set of particles, the scaffold can be configured to release the second bioactive agent so as to create a second desired spatial and/or temporal concentration gradient of the second bioactive agent that is different from the first desired spatial and/or temporal concentration gradient of the first bioactive agent.

In one embodiment, the particles can be melded together by only a portion of a polymeric shell of a particle merging with only a portion of at least one adjacent polymeric shell of a particle. Methods of melding particles together are described herein.

In one embodiment, the bioactive agent contained in a particle can be a growth factor for growing the cells. However, the particles can include any type of bioactive agent. Accordingly, the first characteristic can be a first bioactive agent contained in or disposed on the particles, and the second characteristic can be a second bioactive agent contained in or disposed on the particles. For example, the first bioactive agent can be an osteogenic factor and the second bioactive agent can be a chondrogenic factor.

In one embodiment, the scaffold can include a medium sufficient for growing cells disposed in the pores. The medium can be a cell culture medium. Additionally, the medium can be a body fluid or tissue.

In one embodiment, the scaffold can include a plurality of cells attached to the plurality of particles and growing within the pores. Such cells can include a single type of cell, or a first cell type associated with the first set of particles and a second cell type associated with the second set of particles.

In one embodiment, the scaffold can include a first end and an opposite second end. Accordingly, the first set of particles can have a first bioactive agent, and the first end can have a majority of particles of the first set. Correspondingly, the second set of particles can have no bioactive agent or a second bioactive agent that is different from the first bioactive agent, and the second end having a majority of particles of the second set.

In one embodiment, the present invention can include a method of generating or regenerating tissue in an animal, such as a human. The method can include providing an endoprosthesis having the hard core and polymeric shell microparticles for growing cells. The endoprosthesis can have a plurality of biocompatible particles linked together so as to form a three-dimensional matrix having a plurality of pores defined by and disposed between the particles. Accordingly, the endoprosthesis can include a particle-based scaffold. The plurality of particles can have a surface area sufficient for growing cells within the plurality of pores. The biocompatible particles can be characterized as described herein. Additionally, the method of generating or regenerating tissue can include implanting the endoprosthesis in the animal such that cells grow on the particles and within the pores. This process can be used to grow specific types of cells for growth of tissue, bone, cartilage, or the like.

In one embodiment, the method of generating or regenerating tissue can include any one of the following: introducing a cell culture media into the pores; introducing cells into the pores; and/or culturing the cells such that the cells attach to the particles and grow within the pores.

In one embodiment, the three-dimensional implants can be used for the following:

osteochondral defect repair (in the presence of growth factors with or without cells) and tissue engineering; axonal regeneration; study of chemotaxis in three-dimensions; directed angiogenesis; regeneration of other interfacial tissues such as muscle-bone, skin layers; temporal and spatial control of release of inflammatory and/or immune system modulators in regenerative medicine applications; and any application requiring a biocompatible, biodegradable material with spatial and temporal control over material composition, bioactive signal release, and porosity.

In one embodiment, the shells of the particles can be prepared from substantially any polymer, such as biocompatible, biostable, bioerodable, and/or biodegradable polymers. Examples of such biocompatible polymeric materials can include a suitable hydrogel, hydrophilic polymer, hydrophobic polymer, biostable polymers, biodegradable polymers, bioabsorbable polymers, and monomers thereof. Examples of such polymers can include nylons, poly(alpha-hydroxy esters), polylactic acids, polylactides, poly-L-lactide, poly-DL-lactide, poly-L-lactide-co-DL-lactide, polyglycolic acids, polyglycolide, polylactic-co-glycolic acids, polyglycolide-co-lactide, polyglycolide-co-DL-lactide, polyglycolide-co-L-lactide, polyanhydrides, polyanhydride-co-imides, polyesters, polyorthoesters, polycaprolactones, polyesters, polyanhydrides, polyphosphazenes, poly(phosphoesters), polyester amides, polyester urethanes, polycarbonates, polytrimethylene carbonates, polyglycolide-co-trimethylene carbonates, poly(PBA-carbonates), polyfumarates, polypropylene fumarate, polyp-dioxanone), polyhydroxyalkanoates, polyamino acids, poly-L-tyrosines, poly(beta-hydroxybutyrate), polyhydroxybutyrate-hydroxyvaleric acids, polyethylenes, polypropylenes, polyaliphatics, polyvinylalcohols, polyvinylacetates, hydrophobic/hydrophilic copolymers, alkylvinylalcohol copolymers, ethylenevinylalcohol copolymers (EVAL), propylenevinylalcohol copolymers, polyvinylpyrrolidone (PVP), poly(L-lysine), poly(lactic acid-co-lysine), poly(lactic acid-graft-lysine), polyanhydrides (such as poly(fatty acid dimer), poly(fumaric acid), poly(sebacic acid), poly(carboxyphenoxy propane), poly(carboxyphenoxy hexane), poly(anhydride-co-imides), poly(amides), poly(iminocarbonates), poly(urethanes), poly(organophasphazenes), poly(phosphates), poly(ethylene vinyl acetate) and other acyl substituted cellulose acetates and derivatives thereof, poly(amino acids), poly(acrylates), polyacetals, poly(cyanoacrylates), poly(styrenes), poly(vinyl chloride), poly(vinyl fluoride), poly(vinyl imidazole), chlorosulfonated polyolefins, polyethylene oxide, combinations thereof, polymers having monomers thereof, or the like. In certain preferred aspects, the nano-particles include hydroxypropyl cellulose (HPC), N-isopropylacrylamide (NIPA), polyethylene glycol, polyvinyl alcohol (PVA), polyethylenimine, chitosan, chitin, dextran sulfate, heparin, chondroitin sulfate, gelatin, etc. and their derivatives, co-polymers, and mixtures thereof.

The methods of making the scaffolds from the particles can be changed to include a solvent or solvent system (i.e., media or media system) that is liquid or vapor and that is compatible with the particular polymer of the particle. That is, the solvent or solvent system can be selected to meld the particles together as described herein in either liquid or vapor format. Vapor melding can be especially advantageous to control melding properties. Examples of some solvents can include hexane, benzene, toluene, diethyl ether, chloroform, ethyl acetate, 1,4-dioxane, tetrahydrofuran, dichloromethane, acetone, acetonitrile, dimethylformamide, dimethyl sulfoxide, acetic acid, n-butanol, 2-butanol, 3-butanol, t-butyl alcohol, carbon tetrachloride, chlorobenzene, isopropanol, n-propanol, ethanol, methanol, formic acid, water, cyclohexane, 1,2-dichloroethane, diethyl ether, diethylene glycol, diglyme, dimethyl ether, dioxane, ethylene glycol, glycerin, heptane, hexamethylphosphoramide, hesamethylphosphorous triamide, hexane, nitromethane, pentane, petroleum ether, propanol, pyridine, o-xylene, m-xylene, p-xylene, and the like. Carbon dioxide can also be used as a solvent or media to meld the particles together. Also, solvents known for particular polymers can be used or combined with the solvents described herein.

Fabrication of Polymer Coated Glass Beads

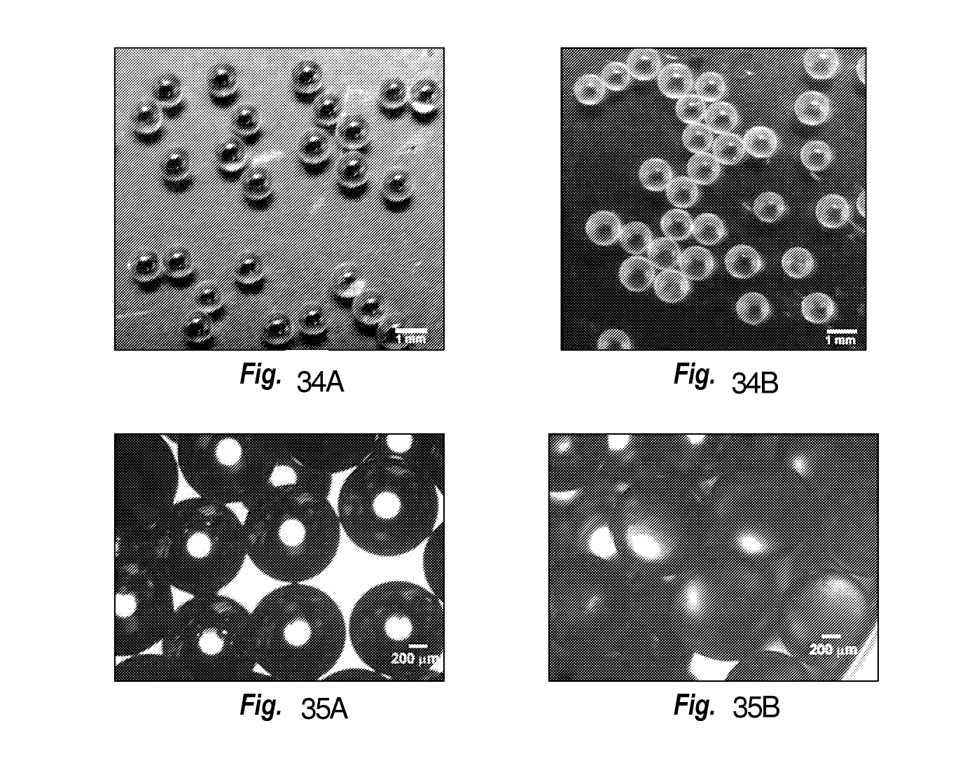

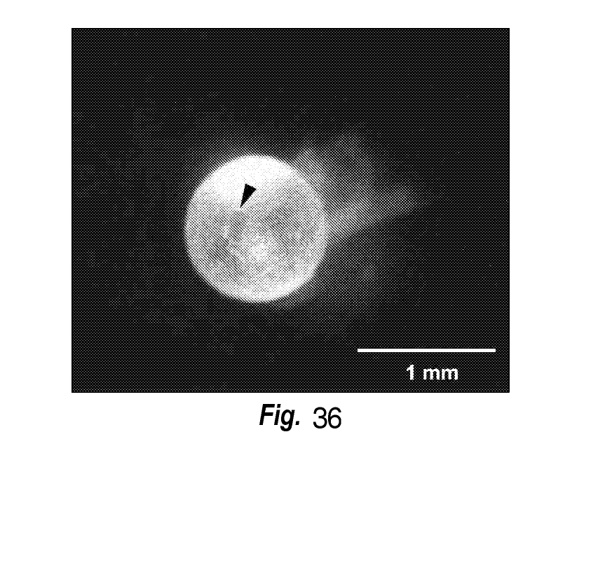

A circulating fluidized bed coating process was used to fabricate spheres with a stiff core and a soft outer layer of polymer. Soda lime glass beads of 1-1.18 mm size (−16+18 mesh size, MO-SCI, Rolla, Mo.) were used as the stiffer core material. A 5% w/v solution of Poly(D,L-lactic-co-glycolic acid) (PLGA) (50:50 lactic acid:glycolic acid, acid end group, MW ˜42,000-44,000 Da) of intrinsic viscosity 0.34-0.36 dL/g or polycaprolactone (PCL) (ester end group of intrinsic viscosity 1-1.3 dL/g) dissolved in methylene chloride were used to coat the beads. The coating was carried out using a UniGlatt® fluidized-bed coater. Prior to coating, the beads were fluidized in the chamber for 10 minutes until the outlet temperature of the coater reached 40° C. The process parameters for the PLGA coating were as follows: atomization pressure 1.5 bar; inlet temperature 45-50° C.; outlet temperature 40-42° C.; fluidizing air setting 2.2-3.0 kPa; relative humidity 3% and spraying rate 7 g/minute. The process parameters for the PCL coating were as follows: atomization pressure 1.5 bar; inlet temperature 40-45° C.; outlet temperature 38-40° C.; fluidizing air setting 2.3-2.6 kPa; relative humidity 3% and spraying rate 2.5 g/minute. The coating process was continuously monitored through the glass window of the coating device to ensure a smooth fluidization of bed. At intervals, the coated beads were fluidized in air for 10 minutes to remove the residual organic solvent and to avoid clustering of the beads. The weight of uncoated glass beads and the coated beads were measured. A total of 8 g of PLGA and 18 g of PCL were coated per 1000 g of glass beads. Photographs and microscopic images of beads before and after coating were taken. The polymer coated beads appeared white/opaque in color when compared to the uncoated transparent glass beads. (FIGS. 34A-34B and 35A-35B). FIG. 34A includes images of uncoated glass beads, and FIG. 34B includes images of glass beads coated with PLGA. FIG. 35A shows a phase contrast image of uncoated glass beads, and FIG. 35B shows a phase contrast image of glass beads coated with PLGA. A scratch was created on the surface of the beads using a scalpel blade and imaged to visualize the coating as shown in FIG. 36, where the arrow points to the scratch on the surface to visualize the coating.

Solvent Vapor Sintering

The PCL coated glass beads were packed in cylindrical polypropylene molds of 7 mm diameter to a height of 10 mm. These molds were exposed to 50 ml of methylene chloride vapors for 3 hrs and 4 hrs in a tightly closed container. The scaffolds were removed from molds and placed in a fume hood overnight to remove the residual vapors. The control scaffold made of PCL 200 micron particles are sintered by CO₂ exposure to an absolute pressure of 690 psi (47.6 bar) at 45° C. for a period of 4 hours followed by depressurization at a rate of ˜0.2 psi/s for 1 hour.

Selective Laser Sintering

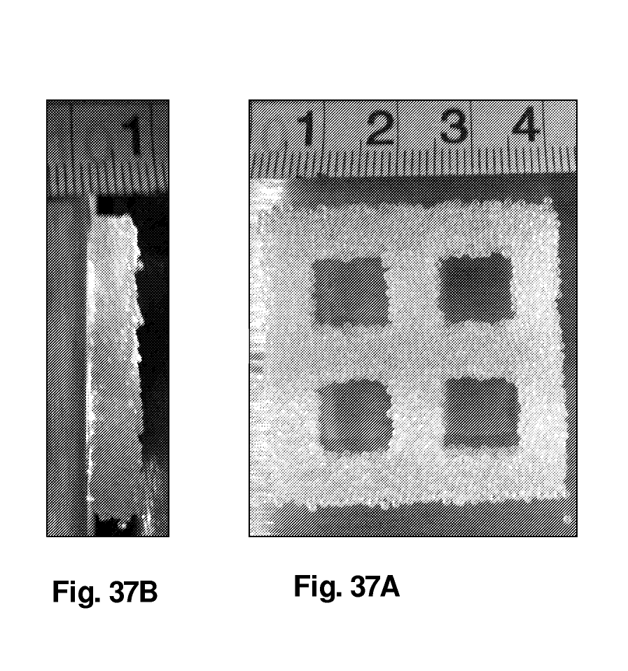

Using an SLS system (Sinterstation 2500plus, DTM, USA) that was modified to accommodate a limited amount of material, settings were tuned in order to achieve the desired laser sintering. The coated beads were placed into the build chamber and the two feed chambers, then the chamber was closed and warmed to 38° C. with the build heater at 18% power and at an inner/outer power ratio of 0.5, and with the feed chamber heaters at 10% power. The chamber was then opened and custom-made heat shields were inserted, and the chamber continued to heat for about 20 additional minutes to a set point temperature of 48° C. The laser power was set at 25 W, with an overlap of 0.3 mm and spacing set at 0.1 mm. The build chamber was set to drop 1.4 mm per layer, and the feed pistons were set to raise 2.8 mm per layer. A part (scaffold) was sintered that had a “window frame” shape, with a 4 cm×4 cm square, with four 1 cm×1 cm “windows” and 0.5 cm wide struts. A build height (part thickness) of 5 mm was achieved as shown in FIGS. 37A-37B. In other similar runs, thicknesses of up to 1.0 cm were achieved, limited only by the available amount of coated beads. FIG. 37A shows a view of a “window pane” formed from the engineered particles and shape sintered by SLS, with a 4×4 cm outer dimension, 1×1 cm windows, and 0.5 cm struts (e.g., frame). FIG. 37B shows a side view of the window pane of FIG. 37A to demonstrate a thickness of 0.5 cm, providing evidence of not only selective laser sintering, but also layering of selective laser sintering.

CO₂ Sintering

Scaffolds were fabricated by exposure to sub-critical levels of CO₂ in a custom-designed stainless steel vessel having a pressure safety rating of 60 bar. Specific amounts of PLGA coated glass beads were loaded into a Teflon molds and exposed to CO₂. The beads were exposed to a CO₂ absolute saturation pressure of 700 bar at 45° C. for 4 hours followed by depressurization at the rate of 0.101 psi/s for 1 hour.

Heat Sintering

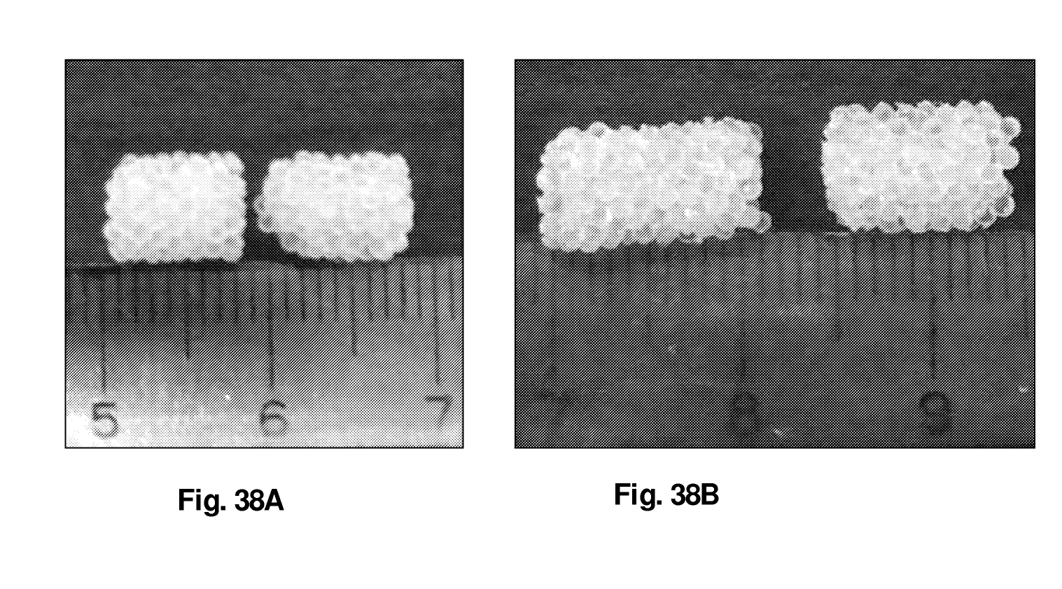

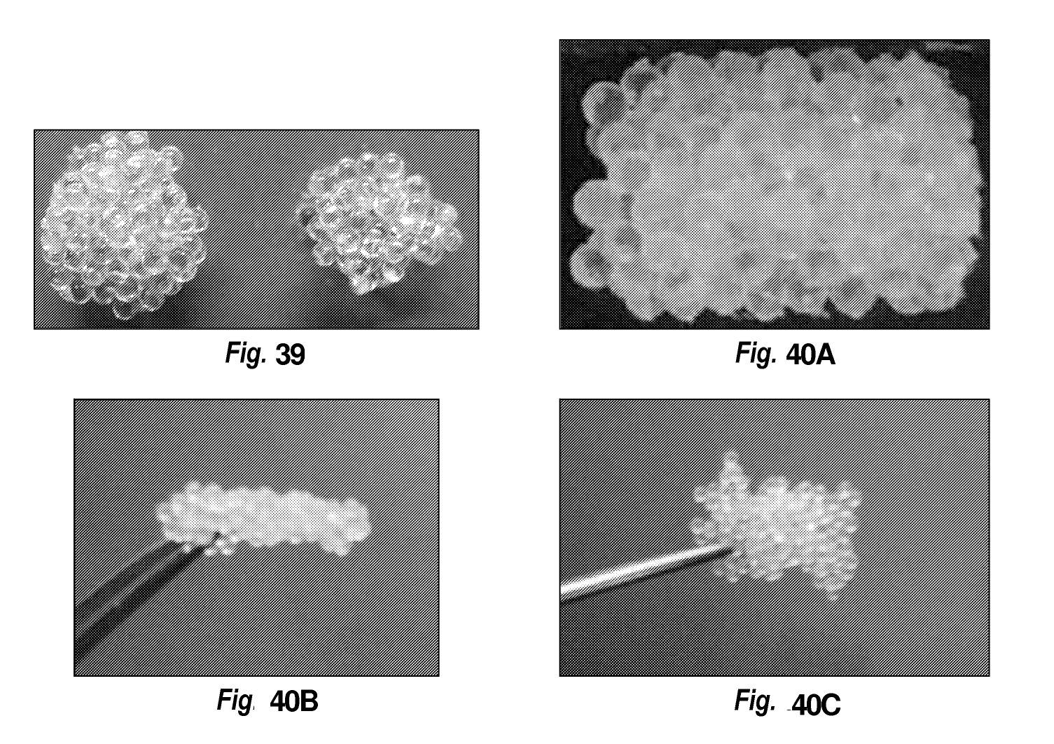

The PLGA and PCL coated beads were packed in molds and sintered by heat at 56° C. for 4 hours. Three-dimensional scaffolds could be fabricated by sintering the outer polymer layer of each beads by all of these methods as shown in FIGS. 38A-38B and 39. FIG. 38A shows PCL coated glass beads sintered by methylene chloride vapors at 3 hours of sintering and FIG. 38B shows 4 hours of sintering. FIG. 39 shows PLGA coated glass beads sintered by sub-critical CO₂.

Mechanical Characterization of 3 Dimensional Scaffolds Sintered Using Methylene Chloride Vapors

The compressive modulus of 3 dimensional scaffolds was performed using a uniaxial testing apparatus (Instron Model 5848, Canton, Mass.) with a 50 N load cell. Tare-loaded (0.05 N) constructs were compressed at a rate of 1 mm/minute in a dry state and immersed in phosphate buffered saline (PBS) at 37° C. Moduli of elasticity were calculated from the initial linear regions of the stress-strain curves. (Table 1) The beads remained sintered together after compression to 80% of its initial height. (FIGS. 40A-40C). FIG. 40A shows a scaffold before compression. FIG. 40B shows a lateral view of the scaffold of FIG. 40A after compression. FIG. 40C shows cross sectional view after compression.

TABLE A1
Compressive elastic modulus of polymer coated beads

| | | Modulus (Pa) | Modulus (Pa) |
|---|---|---|---|
| Bead Type | Testing condition | 3 hour sintering | 4 hour sintering |
| PCL coated glass | Dry state (RT) | Range (2 − 5 × 10⁷) | Range (1 − 2 × 10⁸) |
| beads | (n = 3) | Mean (2.5 × 10⁷) | Mean (1.6 + 0.53 × 10⁸) |
| PCL coated glass | Wet state | — | Range (1 − 9 × 10⁶) |
| beads | (37° C., PBS) (n = 6) | | Mean (3.66 + 2.9 × 10⁶) |
| PCL microspheres | Wet state | — | Mean (45.6 ± 26.3 × 10³) |
| 200 microns | (37° C., PBS) (n = 5) | | |

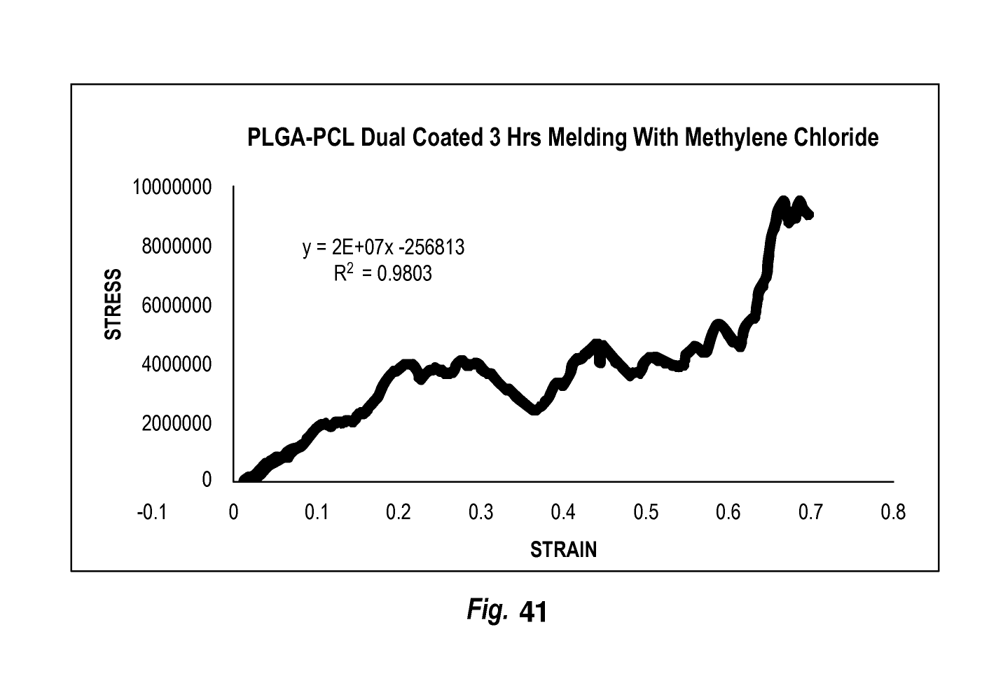

in FIG. 41 shows a graph of stress-strain data obtained for PLGA-PCL dual coated microspheres melded with methylene chloride for 3 hours. The test was done with an anvil height of 7.625 and standard procedures.

Additionally, hUCMSCs were seeded on the sintered scaffolds and cultured for a period of 24 hrs and one week. Cell viability assay was carried out using Live Dead assay kit containing calcien AM and ethidium bromide dye. The constructs were imaged using confocal microscope. hUCMSCs passage number 1 were used for the live/dead assay. Cells were seeded at a density of 1 million cells per scaffold. The cells were cultured in DMEM (Low Glucose) containing 1% Pencillin/Streptomycin, 10% Fetal Bovine Serum (FBS). Live cells showed green fluorescence. Cells were viable on both the 24 hr and 1 week constructs. The cells on the week 1 constructs were properly spread and covered the bead surface and the interstitial space. The results showed that the coated beads provide a favorable non-toxic surface for cell attachment and proliferation.

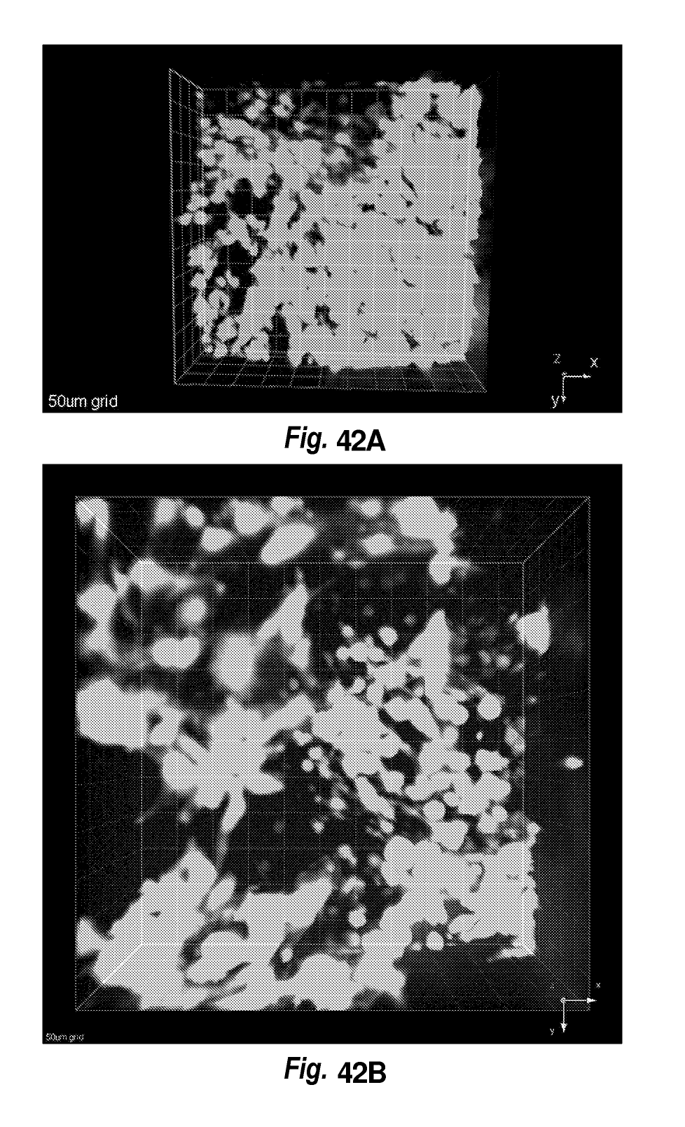

FIGS. 42A and 42B include images of 1 mm engineered particles having the hard core and polymeric shells with live:dead cells at one week, which images are color contrasted to show the cells. The cells are human umbilical cord cells (hUCMSCs) seeded on coated glass particles in a tissue engineering scaffold.

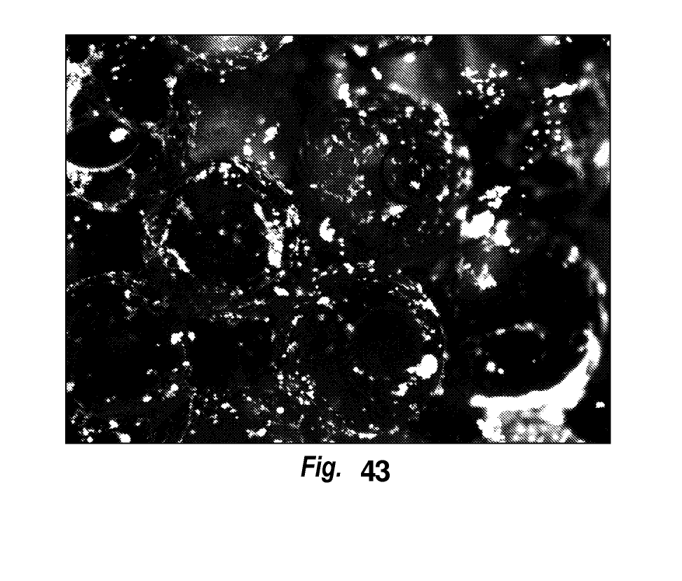

FIG. 43 includes images of 1 mm engineered particles having the hard core and polymeric shells with live:dead cells at 24 hours, which images are color contrasted to show the cells. The cells are human umbilical cord cells (hUCMSCs) seeded on coated glass particles in a tissue engineering scaffold. The spaces between the particles show the porosity (1st level porosity or interstitial space).

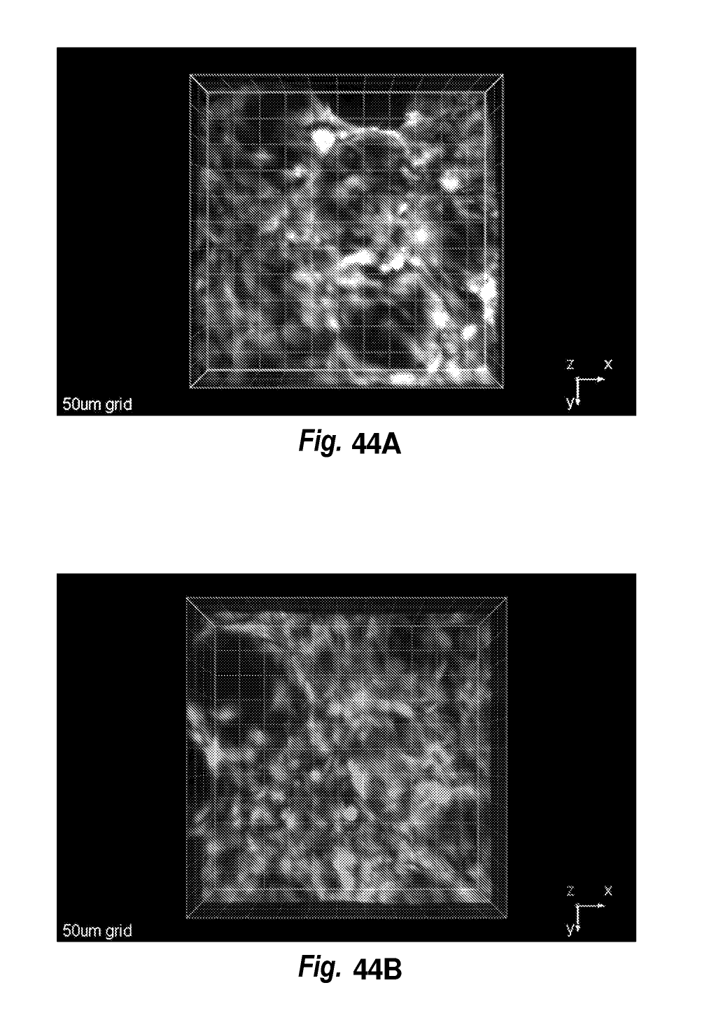

FIGS. 44A and 44B include images of 200 micron engineered particles having the hard core and polymeric shells with live:dead cells at one week, which images are color contrasted to show the cells. The cells are human umbilical cord cells (hUCMSCs) seeded on coated glass particles in a tissue engineering scaffold. The cells are shown to be in the on the particles and in spaces between the particles show the porosity (1st level porosity or interstitial space).

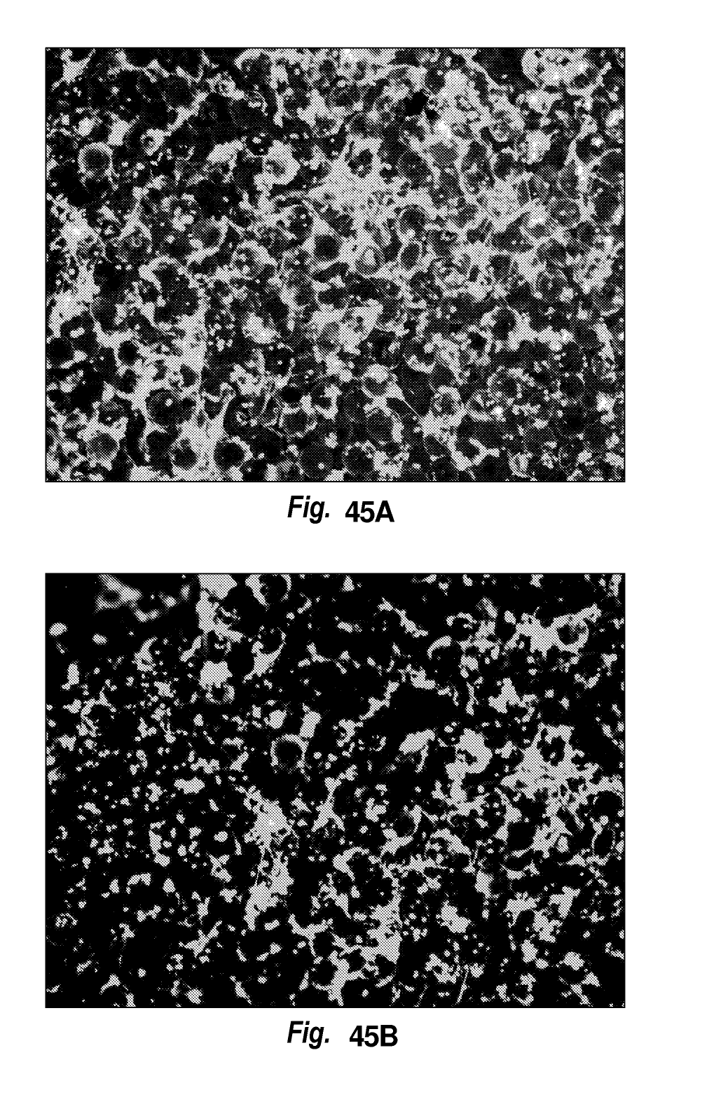

FIGS. 45A and 45B include images of 200 micron engineered particles having the hard core and polymeric shells with live:dead cells at 24 hours, which images are color contrasted to show the cells. The cells are human umbilical cord cells (hUCMSCs) seeded on coated glass particles in a tissue engineering scaffold. The cells are shown to be in the on the particles and in spaces between the particles show the porosity (1st level porosity or interstitial space).

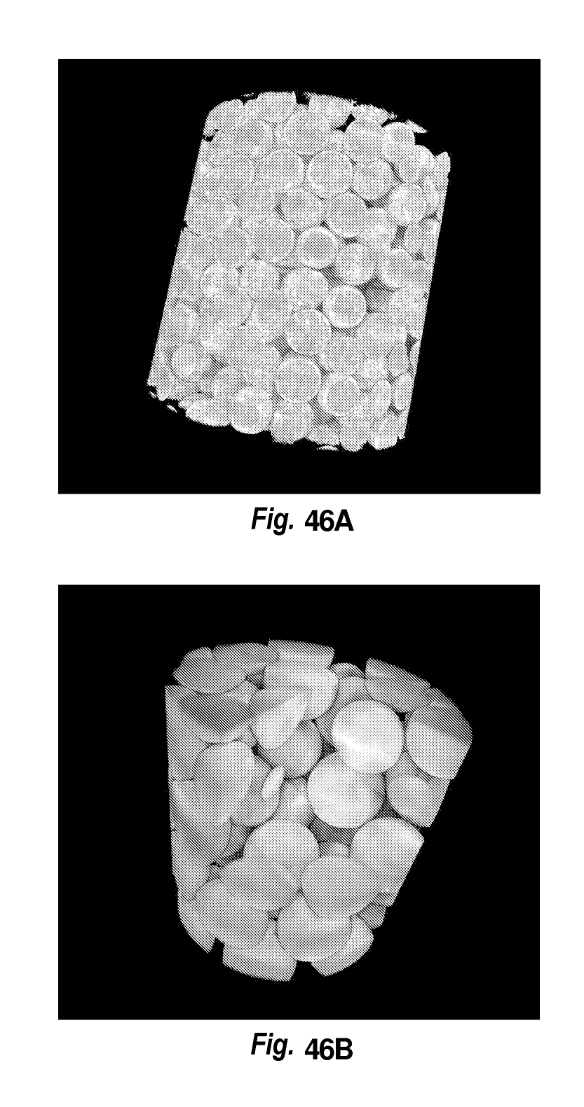

FIG. 46A includes an image of 200 micron engineered particles in a sintered scaffold with interstitial spaces between the particles. FIG. 15B includes an image of 1 mm engineered particles in a sintered scaffold with interstitial spaces between the particles.

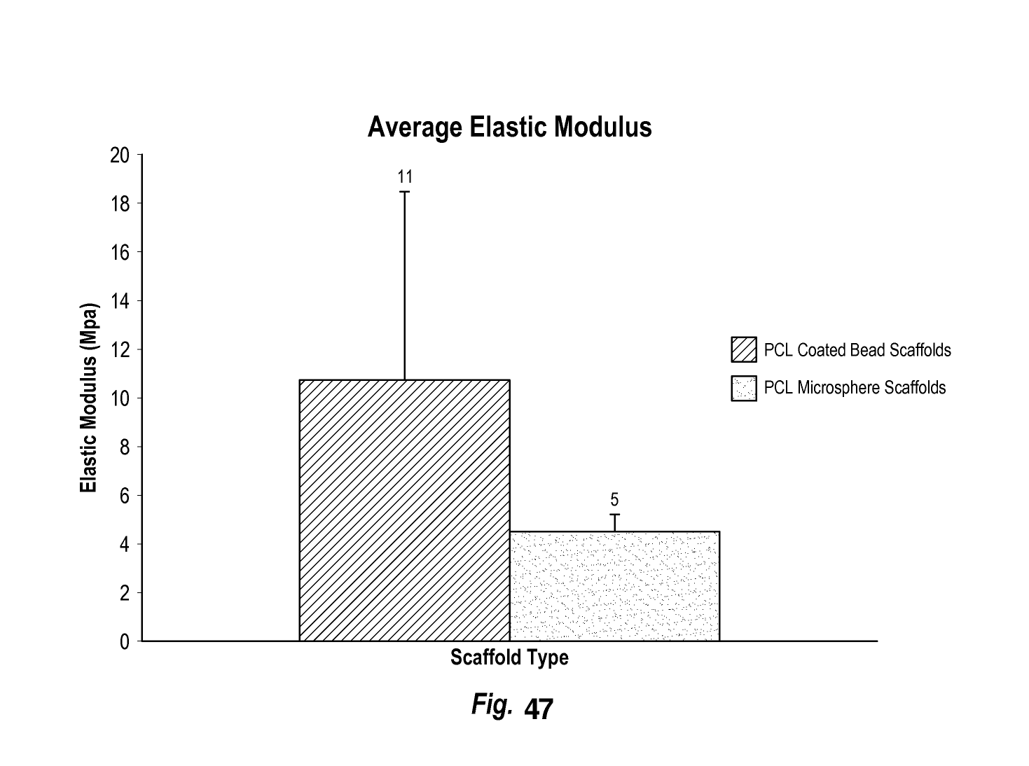

FIG. 47 includes a graph that illustrates the average elastic modulus of PCL coated 200 micron glass bead scaffolds compared to with 200 micron PCL microsphere scaffolds conducted in hydrated conditions. The average elastic modulus of PCL coated glass bead scaffolds (˜11 MPa) is about 2.5 times higher than the average elastic modulus of PCL microsphere scaffolds (4.5 MPa). Furthermore, the range of modulus of elasticity for coated bead scaffolds is 4-30 MPa and for the microsphere scaffolds is 4-5 MPa. As such, scaffolds having the engineered particles with the hard core and polymeric shell can be tailored across a broad range of stiffness and prepared into scaffolds significantly stiffer than polymeric scaffolds.

Additionally, the scaffolds prepared from the engineered particles having the hard core and polymeric shell may be modified to be at least an order of magnitude higher stiffer than scaffolds with just polymeric microspheres. Additionally, the coating thickness and/or particle dimension (e.g., diameter) may be modulated in order to obtain scaffolds that have even greater stiffness. Moreover, the higher degree of stiffness from the engineered particles and the lower degree of stiffness of the regular polymeric microspheres allows for the formation of stiffness gradients and hard stiffness changes at interfacial transitions between the different types of microspheres.

A particle can include a hard core, and one or more polymeric shells encapsulating the core. The hard core can include hydroxyapatite, tricalcium phosphate, calcium carbonate, titanium dioxide, or combinations thereof. The one or more polymeric shells includes a biocompatible polymer, such as polycaprolactone, poly(glycolic acid) (PGA), poly(lactic acid) (PLA), poly(lactic-co-glycolic acid) (PLGA), derivatives thereof, salts thereof, or combinations thereof. The particle can be a nanosphere or a microsphere. One or more bioactive agents can be encapsulated in or located on the one or more polymeric shells. One or more growth factors can be encapsulated in or located on the one or more polymeric shells. The growth factors include insulin-like growth factor-I (IGF-I), a transforming growth factor, TGF-beta1, TGF-beta3, a bone morphogenetic protein, BMP-2, BMP-7, or combinations thereof.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims. All references recited herein are incorporated herein by specific reference in their entirety: U.S. patent application Ser. No. 13/591,087 filed Aug. 21, 2012; U.S. patent application Ser. No. 12/248,530 filed Oct. 9, 2008 now U.S. Pat. No. 8,277,832; and U.S. Provisional Patent Application No. 61/594,568 filed Oct. 10, 2007.

1. A biocompatible implant configured as a tissue engineering scaffold comprising:
a plurality of biocompatible particles linked together so as to form a three-dimensional matrix having a plurality of pores defined by and disposed between the particles, said plurality of particles having a surface area sufficient for growing cells within the plurality of pores, said plurality of biocompatible particles comprising:
a first set of particles having a first characteristic, the first set of particles having a first predetermined spatial distribution with respect to a first end of the three-dimensional matrix, the first set of particles having a core and one or more polymeric shells, the core and polymeric shells being different materials; and
a second set of particles having a second characteristic that is different from the first characteristic, the second set of particles having a second predetermined spatial distribution with respect to a second end of the three-dimensional matrix and that is different from the first predetermined spatial distribution with respect to the three-dimensional matrix.

2. A biocompatible implant as in claim 1, wherein the first predetermined spatial distribution is distinct from and adjacent to the second predetermined spatial distribution.

3. A biocompatible implant as in claim 1, wherein the first predetermined spatial distribution forms a first concentration gradient of the first set of particles and the second predetermined spatial distribution forms a second concentration gradient of the second set of particles.

4. A biocompatible implant as in claim 1, wherein the three dimensional matrix comprises:
a first portion having a majority of particles of the first set; and
a second different portion having a majority of particles of the second set.

5. A biocompatible implant as in claim 1, wherein the three-dimensional matrix comprises:
a first portion having a majority of particles of the first set;
a second portion having a majority of particles of the second set; and
a third portion disposed between the first portion and the second portion, wherein the first predetermined spatial distribution in the third portion forms a first concentration gradient of the first set of particles and the second predetermined spatial distribution in the third portion forms a second concentration gradient of the second set of particles.

6. A biocompatible implant as in claim 1, the first and second characteristics are independently selected from the group consisting of the following: composition; polymer; particle size; core size; shell thickness; shell layer thickness; particle size distribution; type of bioactive agent; type of bioactive agent combination; bioactive agent concentration; amount of bioactive agent; rate of bioactive agent release; mechanical strength; flexibility; rigidity; color; radiotranslucency; or radiopaqueness.

7. A biocompatible implant as in claim 1, said scaffold further comprising live cells and a medium sufficient for growing the cells disposed in the pores.

8. A biocompatible implant as in claim 1, said scaffold further comprising a plurality of live cells attached to the plurality of particles.

9. A biocompatible implant as in claim 1, said scaffold being further characterized by the following:
a first cell type associated with the first set of particles; and
a second cell type associated with the second set of particles.

10. A biocompatible implant as in claim 1, a first end of the scaffold having a majority of the first set of particles and an opposite end of the scaffold having a majority of the second set of particles.

11. A biocompatible implant as in claim 1, wherein the core is harder than the shells.

12. A biocompatible implant as in claim 11, wherein the core is hydroxyapatite, bioglass, or tricalcium phosphate derivative and the shells are polymeric.
